(19) United States
(12) Patent Application Publication
(10) Pub. No.: US 2024/0417718 A1
(43) Pub. Date: Dec. 19, 2024

(54) SYSTEMS AND METHODS FOR LIBRARY PREPARATION ADAPTERS

(71) Applicant: Ultima Genomics, Inc., Fremont, CA (US)

(72) Inventors: Tyson CLARK, Newark, CA (US); Tommie Lloyd LINCECUM, JR., Newark, CA (US); Daniel MAZUR, Newark, CA (US)

(21) Appl. No.: 18/745,437

(22) Filed: Jun. 17, 2024

Related U.S. Application Data
(63) Continuation of application No. PCT/US2022/053537, filed on Dec. 20, 2022.
(60) Provisional application No. 63/292,332, filed on Dec. 21, 2021.
(60) Provisional application No. 63/394,599, filed on Aug. 2, 2022.

(51) Int. Cl. C12N 15/10

(52) U.S. Cl. CPC C12N 15/1093

(57) ABSTRACT

Provided herein are systems, methods, compositions, and kits for library preparation. In some cases, multiple distinct types of adapter molecules may be provided to a template nucleic acid molecule. In some cases, a single type of adapter molecule may be provided to a template molecule. In some cases, multiple distinct types of adapter molecules may be sequentially provided to a template molecule to form multi-adapter template complexes.

### CROSS-REFERENCE

This application is a continuation of International Application No.: PCT/US2022/053537, filed Dec. 20, 2022, which claims the benefit of U.S. Patent Application No. 63/292,332, filed Dec. 21, 2021, and U.S. Patent Application No. 63/394,599, filed Aug. 2, 2022, each of which is incorporated by reference herein in its entirety.

### SEQUENCE LISTING

The instant application contains a Sequence Listing which has been submitted electronically in XML format and is hereby incorporated by reference in its entirety. Said XML copy, created on May 12, 2023, is named 51024-774_601_SL.xml and is 1.36 megabytes in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious efforts.

### SUMMARY

Preparation of libraries for sequencing can require comparatively large amounts of genetic material (e.g., deoxyribonucleic acid (DNA), ribonucleic acid (RNA), etc.) of interest (e.g., from a sample of a subject). This genetic material is, in some cases, difficult to collect or inherently limited in availability (e.g., complementary DNA (cDNA)). Thus, recognized herein is a need for preparing libraries for sequencing in an efficient manner, maximizing use of sample genetic material. Provided herein are systems, methods, compositions, and kits for library preparation that addresses at least the abovementioned needs.

Provided herein, are nucleic acid compositions. In an aspect, a nucleic acid composition comprises a first strand hybridized to a second strand, wherein: a. a biotin is disposed at a 5′ end of the first strand; b. the first strand comprises one or more cleavable moieties within 15 nucleotides of the 5′ end of the first strand; and c. a phosphate is disposed at a 5′ end of the second strand.

In some embodiments, the first strand and the second strand have complementary sequences. In some embodiments, the first strand comprises one or more cleavable moieties within 12 nucleotides of the 5′ end of the first strand. In some embodiments, the first strand comprises one or more cleavable moieties within 10 nucleotides of the 5′ end of the first strand. In some embodiments, the first strand comprises one or more cleavable moieties within 7 nucleotides of the 5′ end of the first strand. In some embodiments, the first strand comprises the one or more cleavable moieties within 5 nucleotides of the 5′ end of the first strand. In some embodiments, the one or more cleavable moieties are selected from the group consisting of uracils, ribonucleotide residues, spacers, methylated nucleotide residues, and abasic sites. In some embodiments, the one or more cleavable moieties comprises one or more uracils. In some embodiments, the one or more uracils comprises 3 or fewer uracils. In some embodiments, a 3′ end of the first strand comprises a protective group. In some embodiments, a 3′ end of the second strand comprises a protective group. In some embodiments, the protective group is protective against exonuclease activity. In some embodiments, the protective group is a phosphorothioate. In some embodiments, the nucleic acid composition further comprises a double-stranded insert molecule ligated to the 3′ end of the first strand and the 5′ end of the second strand. In some embodiments, the double-stranded insert molecule comprises a barcode sequence. In some embodiments, the nucleic acid composition further comprises a bead comprising a single-stranded adapter oligonucleotide coupled thereto, wherein the single-stranded adapter oligonucleotide is hybridized to a complex comprising the first strand, the second strand, and the double-stranded insert molecule. In some embodiments, the nucleic acid composition further comprises a streptavidin bound to the biotin.

Provided herein, are nucleic acid compositions. In an aspect, a nucleic acid composition comprises a first strand hybridized to a second strand, wherein: a. the second strand comprises a biotin disposed at the 3′ end; b. the second strand comprises one or more cleavable moieties within 10 nucleotides of the 3′ end; and c. the second strand comprises a phosphate disposed at the 5′ end.

In some embodiments, wherein a. the one or more cleavable moieties within 10 nucleotides of the 3′ end comprises one cleavable moiety; and b. the second strand comprises an additional one or more cleavable moieties within 15 nucleotides of the 5′ end. In some embodiments, the one or more cleavable moieties are selected from the group consisting of uracils, ribonucleotide residues, spacers, methylated nucleotide residues, and abasic sites. In some embodiments, the one or more cleavable moieties comprises one or more uracils. In some embodiments, the one or more uracils comprises 2 uracils. In some embodiments, the one or more uracils comprises 1 uracil. In some embodiments, the first strand has a length of about 60% or less of the length of the second strand. In some embodiments, the first strand has a length of about 50% or less of the length of the second strand. In some embodiments, the second strand comprises a uracil disposed within 10 nucleotides of the 5′ end. In some embodiments, the second strand comprises a uracil disposed within 7 nucleotides of the 5′ end. In some embodiments, the second strand has a length of about 60% or less of the length of the first strand. In some embodiments, the second strand has a length of about 50% or less of the length of the first strand. In some embodiments, a 3′ end of the first strand comprises a protective group. In some embodiments, the protective group is protective against exonuclease activity. In some embodiments, the protective group is a phosphorothioate. In some embodiments, the nucleic acid composition further comprises a double-stranded insert molecule ligated to the 3′ end of the first strand and the 5′ end of the second strand. In some embodiments, the nucleic acid composition further comprises a bead comprising a single-stranded adapter oligonucleotide coupled thereto, wherein the single-stranded adapter oligonucleotide is hybridized to a complex comprising the first strand, the second strand, and the double-stranded insert molecule. In some embodiments, the nucleic acid composition further comprises a streptavidin bound to the biotin. In some embodiments, the double-stranded insert molecule comprises a barcode sequence.

Provided herein, are nucleic acid compositions. In an aspect, a nucleic acid composition comprises a double-stranded adapter comprising a first sequence selected from any one of SEQ ID Nos: 1-19.

In some embodiments, the double-stranded adapter is coupled to a template molecule at a first end of the template molecule. In some embodiments, the template molecule is double-stranded. In some embodiments, the template molecule is further coupled to a double-stranded adapter at a second end of the template molecule, wherein the double-stranded adapter at the second end comprises a sequence selected from any one of SEQ ID Nos: 1-19. In some embodiments, each double-stranded adapter comprises the same sequence. In some embodiments, the double-stranded adapter comprises a first region that is double stranded and a second region that is single-stranded. In some embodiments, the second region is an overhang.

Provided herein, are nucleic acid compositions. In an aspect, a nucleic acid composition comprises a single stranded nucleic acid molecule comprising: a template molecule, a first sequence disposed at a 5′ end of the template molecule and comprising a first plurality of uracils converted from cytosines, and a second sequence disposed at a 3′ end of the template molecule and comprising a second plurality of uracils converted from cytosines, and wherein an unconverted first sequence, which comprises unconverted cytosines corresponding to the first plurality of uracils, and an unconverted second sequence, which comprises unconverted cytosines corresponding to the second plurality of uracils, are reverse complements.

In some embodiments, the nucleic acid composition further comprises a first conversion sequence, comprising (i) a first sequence configured to bind to the first sequence of the single stranded nucleic acid molecule via complementarity. In some embodiments, the first conversion sequence further comprises (ii) a first overhang sequence linked to the first sequence of the first conversion sequence, the first overhang sequence comprising one or more of a primer-binding sequence, a unique molecular identifying sequence, and a barcode sequence. In some embodiments, the nucleic acid composition further comprises a second conversion sequence, comprising (i) a second sequence capable of binding to the second sequence of the single stranded nucleic acid molecule via complementarity. In some embodiments, the second conversion sequence further comprises (ii) a second overhang sequence linked to the second sequence of the conversion sequence, the second overhang sequence comprising one or more of a primer-binding region, a unique molecular identifying region, and a barcode sequence. In some embodiments, the barcode sequence is between 9 and 30 nucleotides in length. In some embodiments, the barcode sequence is between 9 and 11 nucleotides in length. In some embodiments, the first sequence of the single stranded nucleic acid molecule is between 10 and 50 nucleotides in length, and the second sequence of the single stranded nucleic acid molecule is between 10 and 50 nucleotides in length. In some embodiments, the first sequence of the single stranded nucleic acid molecule is between 10 and 30 nucleotides in length, and the second sequence of the single stranded nucleic acid molecule is between 10 and 30 nucleotides in length. In some embodiments, the first sequence of the single stranded nucleic acid molecule is between 10 and 15 nucleotides in length, and the second sequence of the single stranded nucleic acid molecule is between 10 and 15 nucleotides in length. In some embodiments, the first sequence of the single stranded nucleic acid molecule is between 20 and 50 nucleotides in length, and the second sequence of the single stranded nucleic acid molecule is between 20 and 50 nucleotides in length. In some embodiments, first sequence comprises a first plurality of uracils, and the second sequence comprises a second plurality of uracils. In some embodiments, the first plurality of uracils is above a threshold number of uracils. In some embodiments, the second plurality of uracils is above a threshold number of uracils. In some embodiments, the threshold number of uracils is between 2 and 12 uracils. In some embodiments, the first plurality of uracils is at least a percentage of the length of the first sequence; and the second plurality of uracils is at least the percentage of the length of the second sequence. In some embodiments, the percentage is about 20%. In some embodiments, the first sequence and or second sequence comprises at least one cytosine residue. In some embodiments, the first sequence or the second sequence does not comprise a homopolymer sequence. In some embodiments, the unconverted first sequence is selected from the group of SEQ ID Nos: 1-8, and the unconverted second sequence is selected from the group of SEQ ID Nos: 9-19.

Provided herein, are methods for processing a nucleic acid molecule. In an aspect, a method for processing a nucleic acid molecule comprises a) providing a reaction mixture, comprising: i) a plurality of template molecules; and ii) a plurality of double-stranded adapters, each comprising a first unconverted sequence hybridized to a second unconverted sequence; b) attaching a double-stranded adapter of the plurality of double-stranded adapters to each of a first end and a second end of a subset of template molecules from the plurality of template molecules, thereby providing a plurality of double-stranded template-adapter complexes; and c) exposing the plurality of double-stranded template-adapter complexes to conditions sufficient to convert one or more unmethylated cytosine residues to uracil residues in double-stranded adapters of the plurality of double-stranded template-adapter complexes, thereby providing a plurality of single-stranded template-adapter molecules.

In some embodiments, the method further comprises d) performing an amplification reaction using the plurality of single-stranded template-adapter molecules and a plurality of additional pair of adapters comprising first additional adapters and second additional adapters, wherein a first additional adapter of the first additional adapters comprises a first cleavable moiety and a first reactive moiety and a second additional adapter of the second additional adapters comprises a second cleavable moiety, thereby providing template-double-adapter molecules. In some embodiments, a double-stranded adapter of the plurality of double-stranded adapters comprises an overhang region; and the attaching of (b) comprises hybridizing the plurality of double-stranded adapters to the plurality of template molecules and performing a ligation reaction. In some embodiments, the overhang region is disposed at a 3′ end of the double-stranded adapter. In some embodiments, the ligation reaction is performed using a ligase. In some embodiments, the ligation reaction is performed using a ligase and a polymerase. In some embodiments, the first cleavable moiety and the second cleavable moiety are each selected from the group consisting of uracils, ribonucleotide residues, spacers, methylated nucleotide residues, and abasic sites. In some embodiments, at least 75% of the plurality of double-stranded template-adapter complexes are converted into single-stranded template-adapter molecules. In some embodiments, at least 85% of the plurality of double-stranded template-adapter complexes are converted into single-stranded template-adapter molecules. In some embodiments, at least 95% of the plurality of double-stranded template-adapter complexes are converted into single-stranded template-adapter molecules. In some embodiments, the template molecules are double-stranded. In some embodiments, the exposing of (c) converts first unconverted sequences and second unconverted sequences to first converted sequences and second converted sequences, respectively. In some embodiments, in the exposing of (c), prior to providing a plurality of single-stranded template-adapter molecules, the first converted sequences dissociate from the second converted sequences. In some embodiments, the method further comprises sequencing the template-double-adapter molecules. In some embodiments, the exposing of (c) comprises bisulfite conversion. In some embodiments, the exposing of (c) comprises EM-seq. In some embodiments, the first unconverted sequence is selected from the group of SEQ ID Nos: 1-8, and the second unconverted sequence is selected from the group of SEQ ID Nos: 9-19.

Provided herein, are kits. In an aspect, a kit comprises at least 96 non-naturally occurring nucleic acid adapter molecules, each comprising a different sequence selected from any one of SEQ ID NOs: 77-268.

In an aspect, a kit comprises at least 96 non-naturally occurring nucleic acid adapter molecules, each comprising a different sequence selected from any one of SEQ ID NOs: 269-460.

In an aspect, a kit comprises at least 96 non-naturally occurring nucleic acid adapter molecules, each comprising a different sequence selected from any one of SEQ ID NOs: 461-652.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIGS. 3A-3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule.

FIGS. 7A-7C illustrate multiplexed stations in a sequencing system.

FIG. 8A illustrates a non-limiting example schematic of library molecule preparation.

FIG. 8B illustrates a non-limiting example schematic of library molecule preparation using methylation-specific adapters. FIG. 8B discloses SEQ ID NOS 5, 653, 654, 655, 655, 654, 654 and 655, respectively, in order of appearance.

FIG. 8C illustrates a non-limiting example of library molecule preparation using methylated, partially single-stranded adapters. FIG. 8C discloses SEQ ID NOS 656, 657, 657 and 656, respectively, in order of appearance.

FIG. 9 illustrates a non-limiting example of library molecule preparation.

FIG. 10A illustrates non-limiting examples of adapter constructs.

FIG. 10B illustrates a non-limiting example of library preparation using adapters tagged with 3′ biotin.

FIG. 10C illustrates a non-limiting bead-bound adapter-template construct prior to ePCR.

FIG. 10D illustrates a non-limiting free adapter-template construct prior to emulsion polymerase chain reaction (ePCR).

FIG. 10E illustrates a non-limiting adapter molecule comprising both a common sequence and a randomized or unique sequence (e.g., a barcode or unique molecular identifier (UMI)).

FIG. 10F illustrates a non-limiting adapter molecule for PCR-free sequencing.

### DETAILED DESCRIPTION

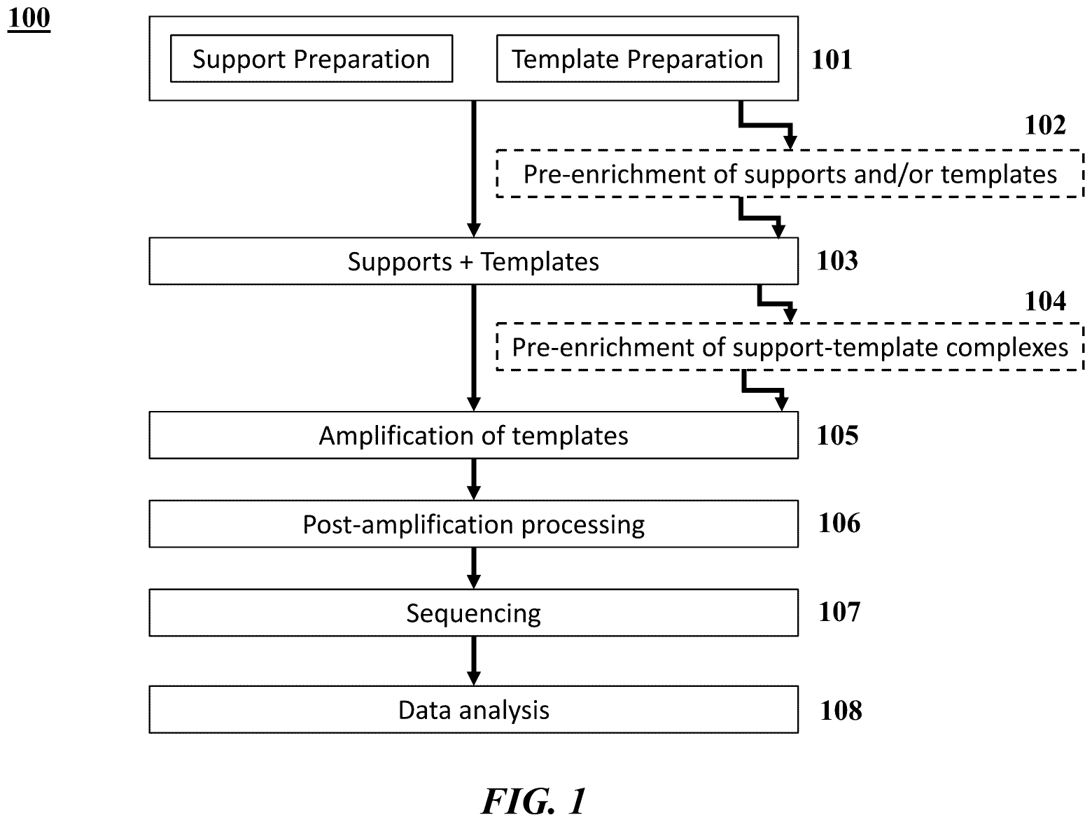

Provided herein are devices, systems, methods, compositions, and kits for library preparation. Such devices, systems, methods, compositions, and kits can be applied alternatively or in addition to sequencing operations described with respect to sequencing workflow 100 of FIG. 1. In addition, such devices, systems, methods, compositions, and kits can be applied alternatively or in addition to template preparation operations described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a native sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid may comprise A nucleic acid can comprise a sequence of four natural nucleotide bases: adenine (A); cytosine (C); guanine (G); and thymine (T) (uracil (U) for thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “terminator” as used herein with respect to a nucleotide may generally refer to a moiety that is capable of terminating primer extension. A terminator may be a reversible terminator. A reversible terminator may comprise a blocking or capping group that is attached to the 3-oxygen atom of a sugar moiety (e.g., a pentose) of a nucleotide or nucleotide analog. Such moieties are referred to as 3-O-blocked reversible terminators. Examples of 3-O-blocked reversible terminators include, for example, 3′-ONH₂ reversible terminators, 3-O-allyl reversible terminators, and 3-O-aziomethyl reversible terminators. Alternatively, a reversible terminator may comprise a blocking group in a linker (e.g., a cleavable linker) and/or dye moiety of a nucleotide analog. 3-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA, or a cDNA derived from the mRNA, or another derivative thereof. In another example, the analyte can be a protein, and the template nucleic acid is an oligonucleotide that is conjugated to an antibody that binds to the protein, or derivative thereof. Sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., A base-containing solution) to a sequencing reaction space at a first time point and (ii) providing a nucleotide-containing reagent (e.g., G-base containing solution) to a sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of canonical base types (A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:
(e.g., [A T G C A T G C A T G A T G A T G A T G C A T G C]). Such one-dimensional matrix or linear array of bases in the flow order may also be referred to herein as a “flow space.” A flow order may have any number of nucleotide flows. A “flow position,” as used herein, generally refers to the sequential position of a given nucleotide flow in the flow space. A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow cycle, consecutively or non-consecutively. Accordingly, the term “flow cycle order,” as used herein, generally refers to an order of flow cycles within the flow order and can be expressed in units of flow cycles. For example, where [A T G C] is identified as a 1′ flow cycle, and [A T G] is identified as a 2^{nd} flow cycle, the flow order of [A T G C A T G C A T G A T G A T G A T G C A T G C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle].

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence-based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical.”

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. Coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction, a magnetic interaction, or a covalent interaction. Similarly, a nucleic acid molecule may be coupled to a particle via a covalent interaction or a via a non-covalent interaction. A coupling between a first object and a second object may comprise a labile moiety, such as a moiety comprising an ester, vicinal diol, phosphodiester, peptide, glycosidic, sulfone, Diels-Alder, or similar linkage. The strength of a coupling between a first object and a second object may be indicated by a dissociation constant, K_{d}, which indicates the inclination of a coupled object comprising a first object and a second object to dissociate into the uncoupled first and second objects and may be expressed as a ratio of dissociated (e.g., uncoupled) objects to coupled objects.

### Sample Processing Methods

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture or be coupled to a template nucleic acid (or processed template nucleic acid). For example, the support may comprise the capture sequence, a primer sequence, a barcode sequence, a sample index sequence, a unique molecular identifier (UMI), a flow cell adapter sequence, an adapter sequence, a binding sequence for any molecule (e.g., splint, primer, template nucleic acid, capture sequence, etc.), or any other functional sequence useful for a downstream operation, or any combination thereof. The oligonucleotide may be single-stranded, double-stranded, or partially double-stranded.

A support may comprise one or more capture entities, where an affinity tag is configured for capture by a capturing entity. An affinity tag may be coupled to an oligonucleotide coupled to the support. An affinity tag may be coupled to the support. For example, the capturing entity may comprise streptavidin (SA) when the affinity tag comprises biotin. In another example, the capturing entity may comprise a complementary capture sequence when the affinity tag comprises a capture sequence (e.g., a capture oligonucleotide that is complementary to the complementary capture sequence). In another example, the capturing entity may comprise an apparatus, system, or device configured to apply a magnetic field when the affinity tag comprises a magnetic particle. In another example, the capturing entity may comprise an apparatus, system, or device configured to apply an electrical field when the affinity tag comprises a charged particle. In some instances, the capturing entity may comprise one or more other mechanisms configured to capture the affinity tag. An affinity tag and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and affinity tag. In some instances, a single affinity tag may be capable of associating with a single capturing entity. Alternatively, a single affinity tag may be capable of associating with multiple capturing entities. Alternatively or in addition, a single capturing entity may be capable of associating with multiple capture entities. The affinity tag may be capable of linking to a nucleotide. Chemically modified bases comprising biotin, an azide, cyclooctyne, tetrazole, and a thiol, and many others are suitable as capture entities. The affinity tag/capturing entity pair may be any combination. The pair may include, but is not limited to, biotin/streptavidin, azide/cyclooctyne, and thiol/maleimide. It will be appreciated that either of the pair may be used as either the affinity tag or the capturing entity. In some instances, the capturing entity may comprise a secondary affinity tag, for example, for subsequent capture by a secondary capturing entity. The secondary affinity tag and secondary capturing entity may comprise any one or more of the capturing mechanisms described elsewhere herein (e.g., biotin and streptavidin, complementary capture sequences, etc.). In some instances, the secondary affinity tag can comprise a magnetic particle (e.g., magnetic bead) and the secondary capturing entity can comprise a magnetic system (e.g., magnet, apparatus, system, or device configured to apply a magnetic field, etc.). In some instances, the secondary affinity tag can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., uracil D glycosylase (UDG), RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethylene glycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adapter sequence of a template nucleic acid and a second subset of oligonucleotides configured to capture a second adapter sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to said support species. When attaching template nucleic acids to supports, only a template nucleic acid comprising a given adapter sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to said support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adapter region of a template nucleic acid or an intermediary primer sequence which can subsequently bind to a complementary nucleic acid sequence within an adapter region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in International Pub. No. WO2020/167656 and International App. No. PCT/US2021/046951, each of which is entirely incorporated herein by reference for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adapter oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adapter sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adapter sequence, the adapter sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprise capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprise cleavable moieties. A cleavable moiety may be strategically placed based on a desired downstream amplification workflow, for example.

In some examples, a library of insert sequences are processed to provide a population of template sequences with identical configurations, such as with identical sequences and/or locations of one or more functional sequences. For example, a population of template sequences may comprise a plurality of nucleic acid molecules each comprising an identical first adapter sequence ligated to a same end. In some examples, a library of insert sequences are processed to provide a population of template sequences with varying configurations, such as with varying sequences and/or locations of one or more functional sequences. For example, a population of template sequences may comprise a first subset of nucleic acid molecules each comprising an identical first adapter sequence at a first end, and a second subset of nucleic acid molecules each comprising an identical second adapter sequence at the second end, where the second adapter sequence is different form the first adapter sequence. In some instances, a population of template sequences with varying configurations (e.g., varying adapter sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adapter sequence) is isolated from a mixture comprising template nucleic acids that do not have the distinct configuration. Alternatively, a template nucleic acid population may be provided to comprise substantially uniform configurations. In some cases, the capture entit(ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit(ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets in an emulsion during emulsion PCR (ePCR or emPCR), or in wells. Emulsion PCR methods are described in further detail in International Pub. No. WO2020/167656 and International App. No. PCT/US2021/046951, each of which is entirely incorporated by reference herein.

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pub. No. 2021/0277464 and International App. No. PCT/US2021/046951, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Any sequencing method described elsewhere herein may be used. In some cases, sequencing by synthesis (SBS) is performed.

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g., [A T G C]), where each nucleotide is not terminated, and where each base is labeled with a same dye (yielding same frequency optical signals). With each flow, other sequencing reagents, e.g., sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%0, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot. Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5-(or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in International Pub. No. WO2019/099886, U.S. Pub. No. 2021/0354126, and U.S. Pub. No. 2021/0277464, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

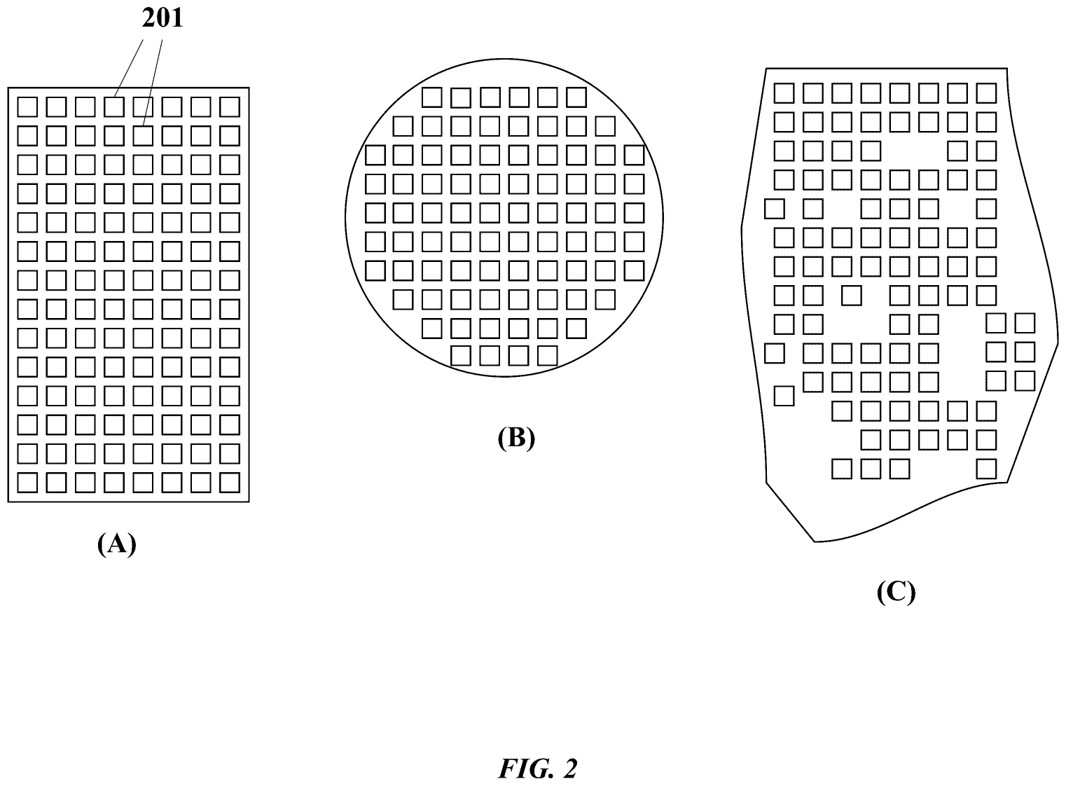

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 m², about 3.75 m², about 4 m², about 4.25 μm², about 4.5 m², about 4.75 m², about 5 m², about 5.5 m², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (m), about 0.2 μm, about 0.25 μm, about 0.3 μm, about 0.4 μm, about 0.5 μm, about 0.6 am, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 am, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 m or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

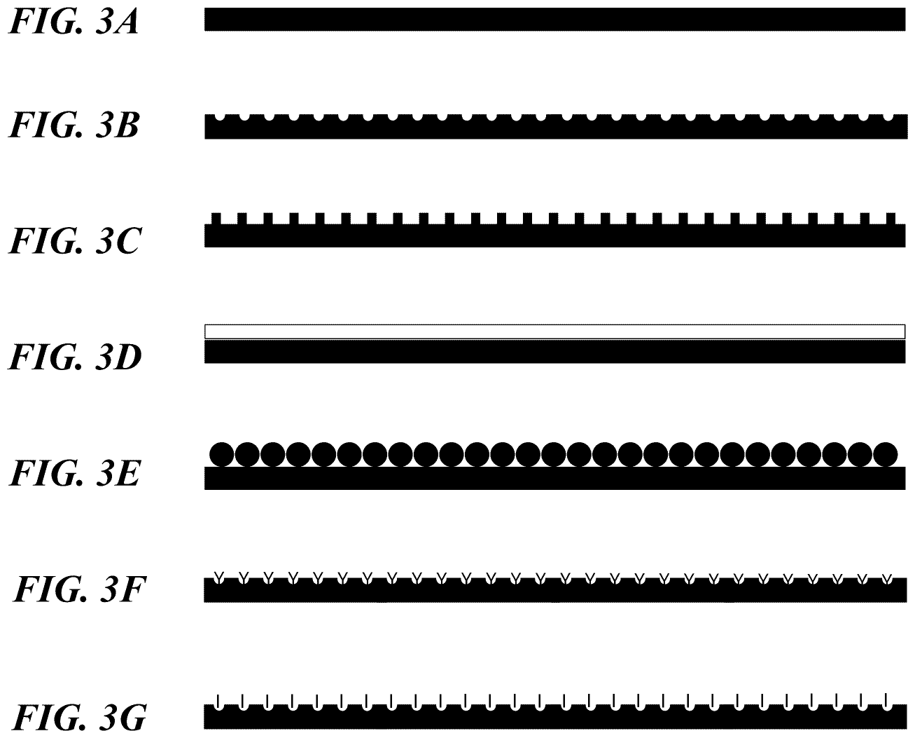

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3% 2%, 10%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

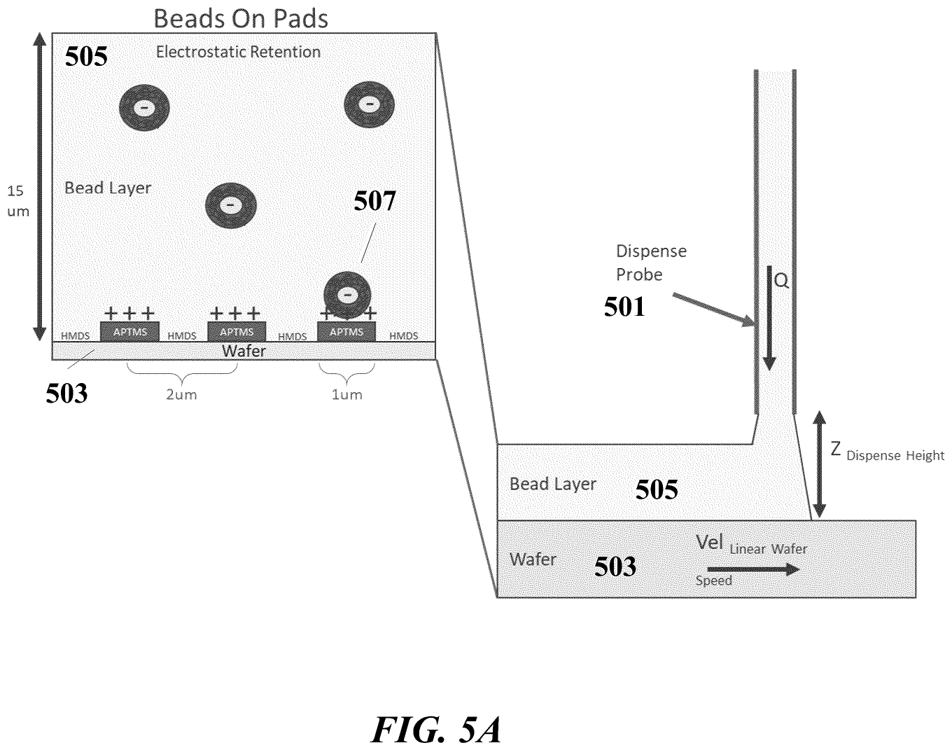

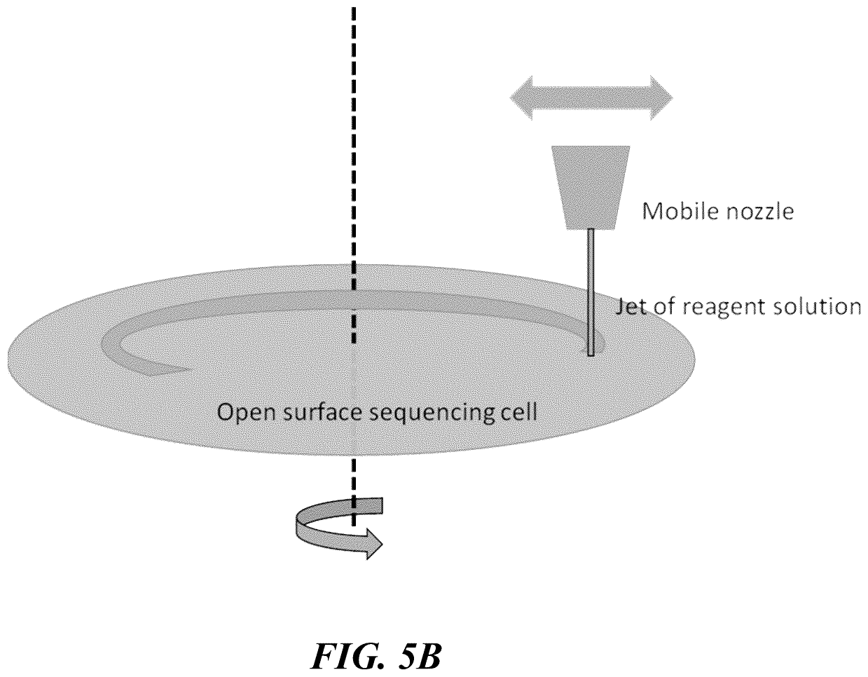

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

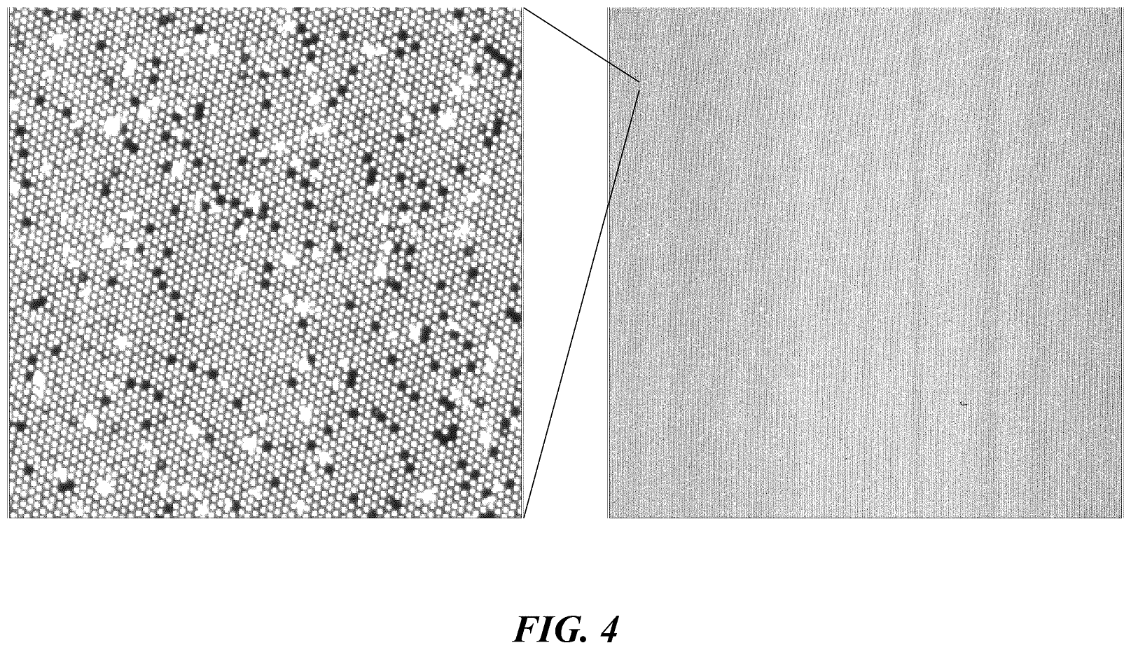

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention, and the beads may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

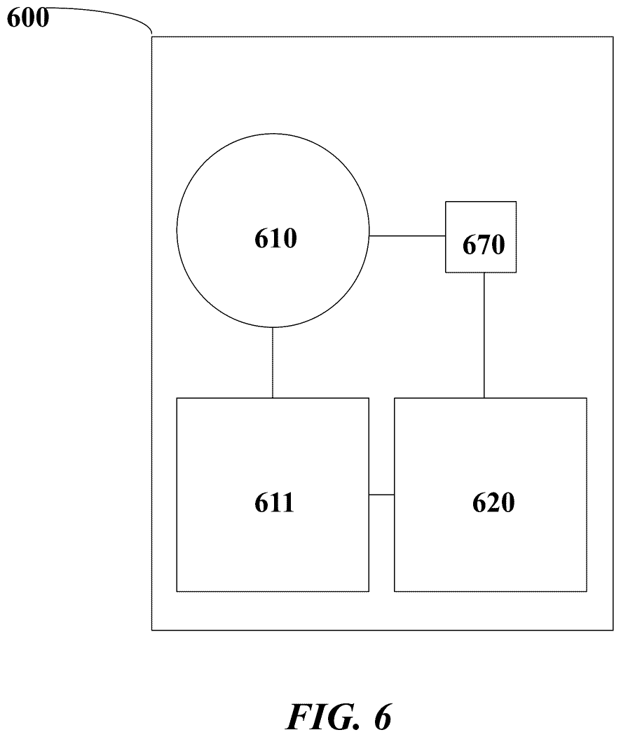

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in International Pub. No. WO2020/118172, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

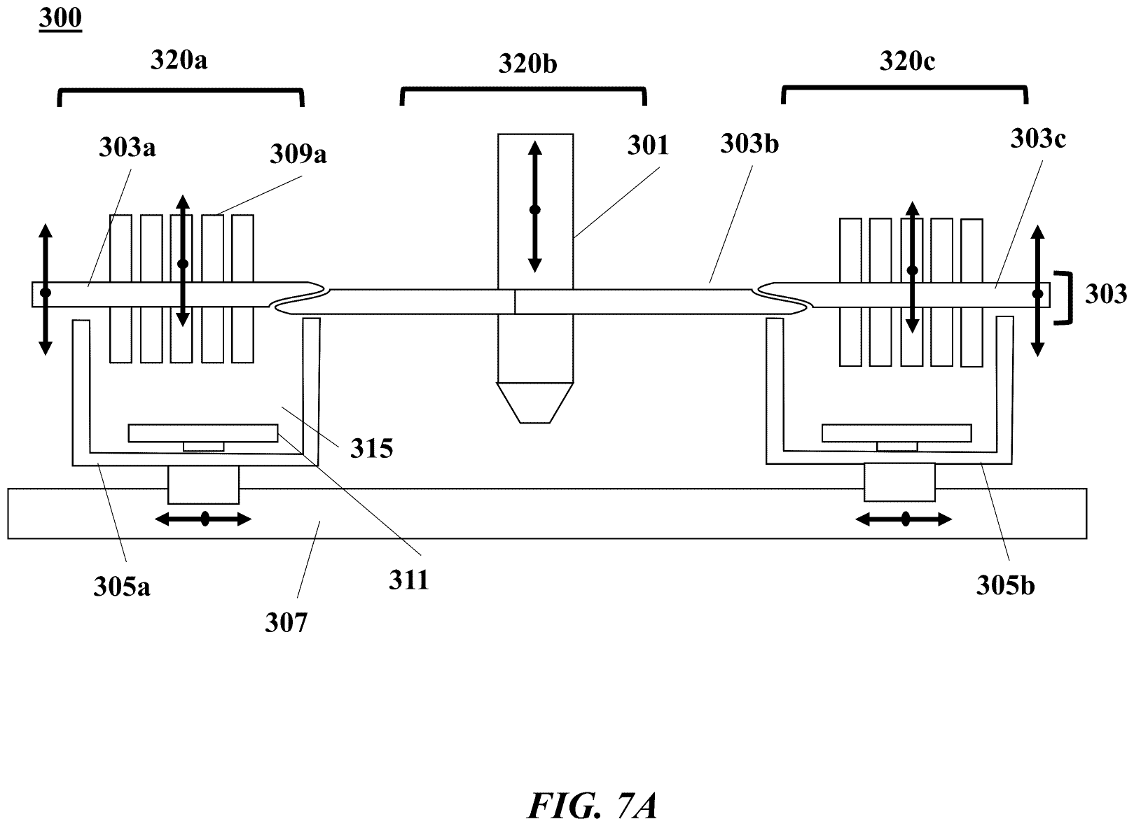

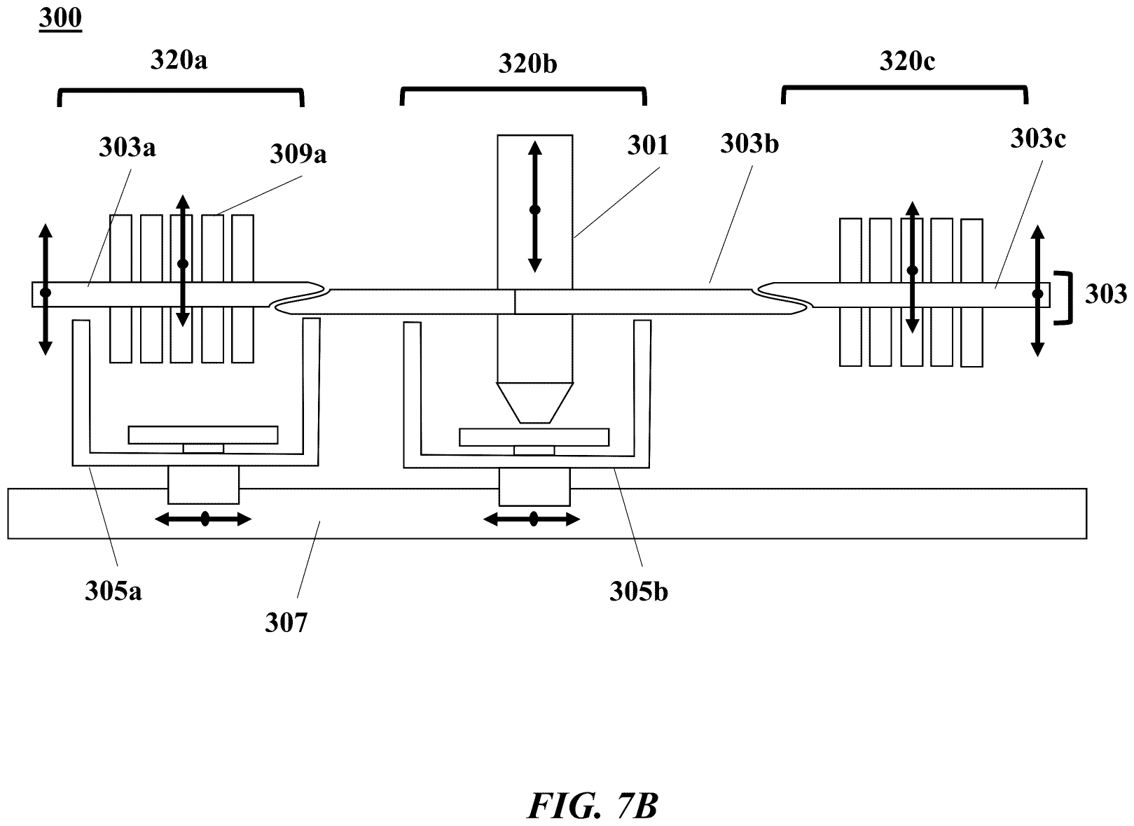

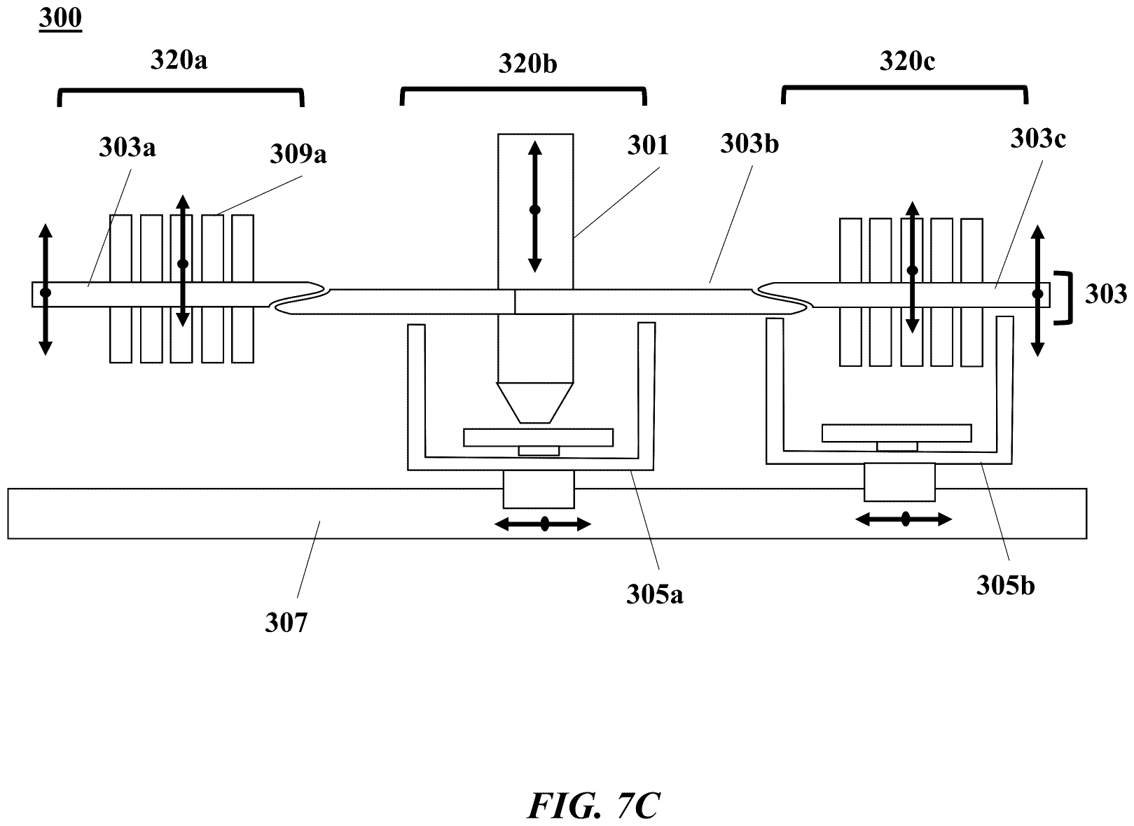

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Single Adapters for Methylation Sequencing

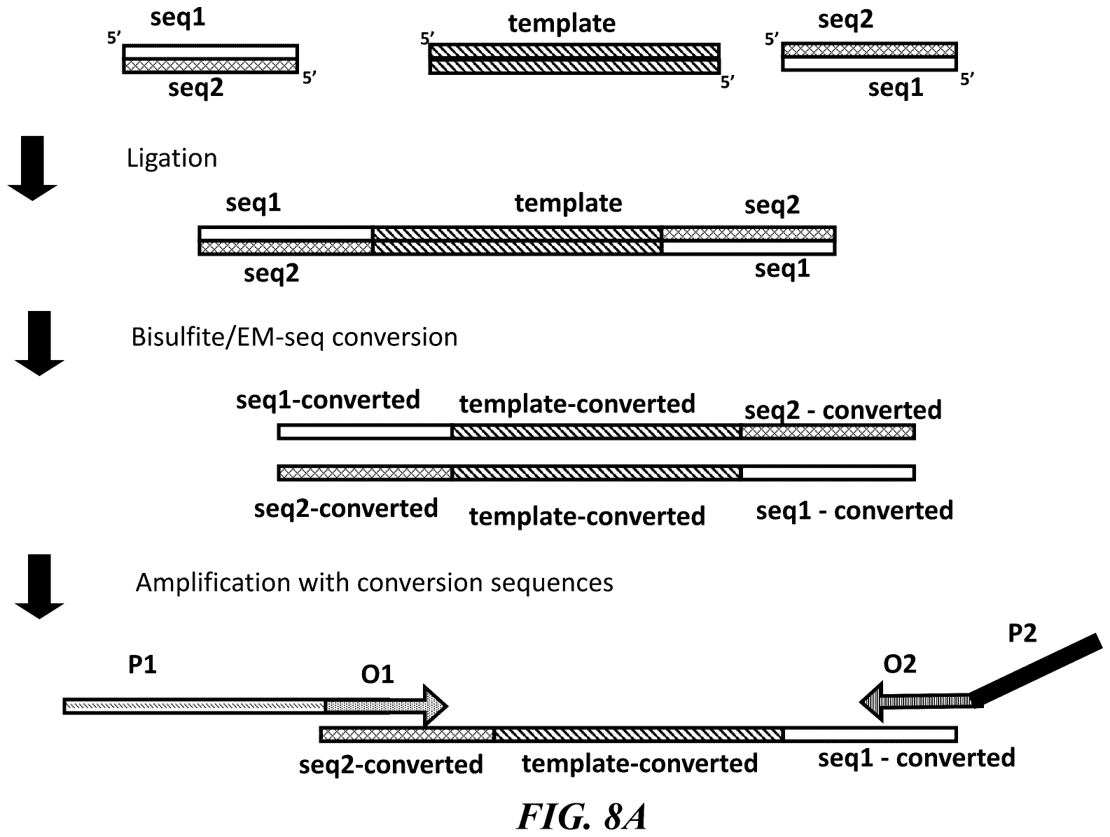

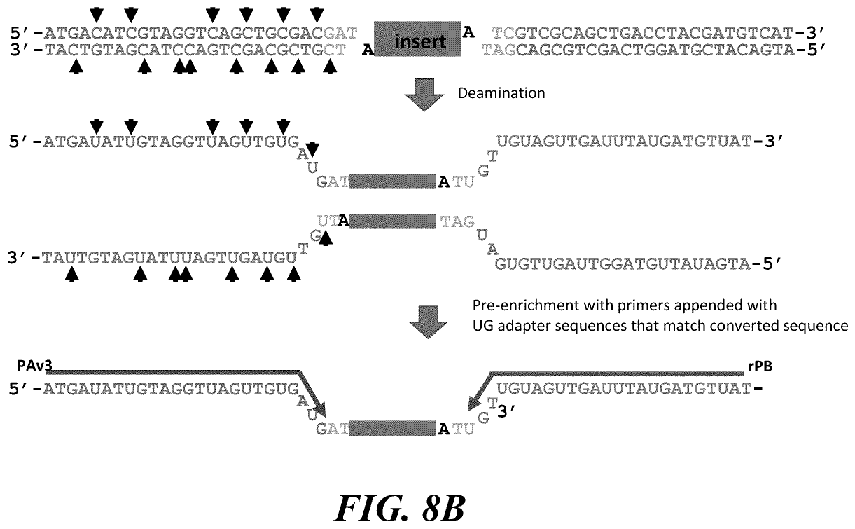

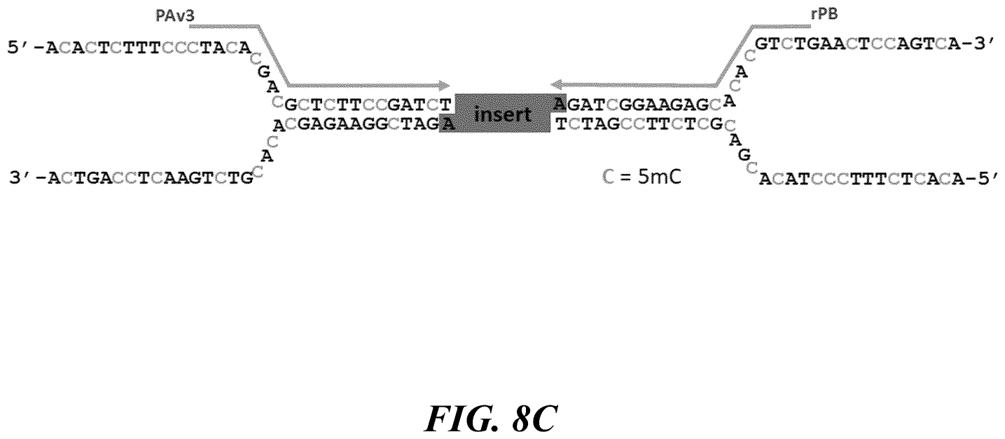

One issue with the construction of libraries for sequencing is the inevitable loss of some sample material during library preparation, especially due to the attachment of adapters to library molecules. For instance, where template molecules are desired to be coupled to a first type of adapter on one end and a second type of adapter on the other end (e.g., where the first and second adapters serve different downstream purposes, such as bead attachment vs. sequencing primer), only about 50% of the resulting library molecules will be ligated to one of each type of adapter (where about 25% of the resulting library molecules will be ligated to the first type of adapter at each end and about 25% of the resulting library molecules will be ligated to the second type of adapter at each end). Thus, there is a significant advantage in terms of library preparation efficiency if a single species of adapter can be used to serve each distinct downstream purpose. The devices, systems, methods, compositions, and kits provided herein may allow for the efficient preparation of template nucleic acid molecules for sequencing (e.g., library preparation for methylation sequencing) by the use of a single adapter species. Example schemes are illustrated in FIGS. 8A-C.

As shown in FIG. 8A, a template molecule is provided with a plurality of double-stranded adapters (e.g., adapters with first sequence seq1 hybridized to second sequence seq2). In some cases, the double-stranded adapters comprise a same nucleic acid sequence (e.g., at least a subset of the plurality of sequencing adapters all comprise a same first sequence seq1 and a same second sequence seq2, where seq1 and seq2 are complementary). The template molecule is coupled to one double-stranded adapter at a first end of the template molecule and another doubled-stranded adapter at a second end of the template molecule. In some cases, the coupling comprises a hybridization between complementary sequences on the template molecule and the double-stranded adapter. For example, in some cases, the double-stranded adapters comprise a first region that is double-stranded and a second region that is single-stranded (e.g., the second region is an overhang). Similarly, the template molecules may comprise a first region that is double-stranded and a second region that is single-stranded (e.g., where the second region is an overhang). In some cases, the overhang sequence of the double-stranded adapter is complementary to the overhang sequence of the template molecule.

A ligation reaction may be performed after coupling of the double-stranded adapters and the template molecule. The ligation reaction may be performed using a ligase (and optionally a polymerase). After the ligation reaction, a double-stranded template-adapter complex is formed, where the double-stranded template-adapter complex comprises, in e.g., 5′ to 3′ orientation, the adapter, the template molecule, and the additional adapter. As there is only one species of adapters in the reaction, nearly 100% of the resulting library molecules will comprise the desired molecular complex.

In some cases, after formation of the double-stranded template-adapter complex molecules, deamination is performed. In some cases, the deamination is bisulfite conversion. In some case, the deamination is Enzymatic Methyl-sequencing (EM-seq) conversion. As a result of deamination, unmethylated cytosines in a double-stranded template-adapter complex are converted to uracils, in both the template and adapter sequences. After the deamination reaction, a double-stranded template-adapter complex is converted into two single-stranded template-adapter complexes, where the single-stranded template-adapter complexes comprise the converted first sequence (e.g., seq1-converted) disposed at the first end of the template molecule, the converted template molecule, and the converted second sequence (e.g., seq2-converted) disposed at the second end of the template molecule. The single-stranded template-adapter complexes arise as a result of the deamination reaction due to the decrease in complementarity between the top and bottom strands of a double-stranded template-adapter complex molecule. That is, the top and bottom strands disassociate or denature from each other as a result of unmethylated cytosines being converted to uracils (e.g., seq1-converted is not complementary to seq2-converted).

In some cases, after deamination the single-stranded template-adapter complex molecules are amplified. In some cases, the amplification is performed with an additional set of adapters (e.g., conversion sequences). The first additional adapter comprises adapter sequence P1 and an overhang sequence O1, where O1 has complementarity to seq2-converted. The second additional adapter comprises adapter sequence P2 and an overhang sequence O2, where O2 has complementarity to seq1-converted. In some cases, the amplification reaction results in template-double-adapter molecules comprising P1, seq2-converted, template, seq1-converted, and P2.

In some cases, the unconverted first sequence (e.g., seq1) comprises one or more unmethylated cytosines. In some cases, seq2 may comprise one or more unmethylated cytosines. In some cases, seq1 comprises one or more unmethylated cytosines while seq2 does not comprise unmethylated cytosines. In some cases, seq2 comprises one or more unmethylated cytosines and seq1 does not comprise unmethylated cytosines. In some cases, the one or more unmethylated cytosines are disposed at a 3′ end of the unconverted first sequence and/or the 3′ end of the second unconverted sequence. In some cases, the template-double-adapter molecules are further analyzed after amplification (e.g., sequencing reaction(s) are performed).

Example adapter sequences for methylation-based library preparation, which may be used as described herein (e.g., seq1 and seq2), are provided in Table 1. Multiple different adapter pairs, where the top strand and the bottom strand have sequence complementarity can be used. For instance, SEQ ID No: 5 may be used as seg1 in conjunction with any one of SEQ ID Nos: 12, 14, and 18 as seq2. As another example, SEQ ID No: 1 and SEQ ID No: 9 have sequence complementarity and may be used together as an adapter pair.

Table 2 includes sequences of the adapter molecules from Table 1 after deamination of the double-stranded template-adapter molecules, where each row in Table 1 corresponds to the same row in Table 2 (e.g., SEQ ID No: 20 is the deaminated sequence of SEQ ID No: 1). In some instances, this deamination is performed by bisulfite treatment or by EM-seq. Table 3 further provides the sequences of primer sequences that may be used for the amplification of the single-stranded template-adapter molecules (e.g., post-deamination) produced as described herein. For library conversion (e.g., where the attachment of additional adapter sequences to the library molecules is desired), additional sequences may be disposed 5′ of the primer sequences (e.g., additional adapter sequences).

For some methods, library molecules may need to be uniquely identifiable. For instance, adapters may further comprise UMIs, barcodes, or other unique sequences. One example for such an adapter construct, for instance with reference to FIG. 8A, would have an unique sequence disposed at the 3′ end of seq1.

TABLE 1
Adapter sequences for methylation sequencing.
C refers to 5-methylcytosine residues.

| SEQ ID No. | Sequence |
|---|---|
| SEQ ID No: 1 | TGCACGTAGCGTACTGCAACGGCATGCAGAT |
| SEQ ID No: 2 | TGCACGTAGCGTACTGCAACGGCATGCACTG |
| | GTACGAT |
| SEQ ID No: 3 | AAGCAGTGGTATCGAACGCAGTCAGCGAT |
| SEQ ID No: 4 | AAGCAGTGGTATCGAACGCAGTCAGCCTGGT |
| | ACGAT |
| SEQ ID No: 5 | ATGACATCGTAGGTCAGCTGCGACGAT |
| SEQ ID No: 6 | ATGACATCGTAGGTCAGCTGCGACCAGAGTA |
| | CTGCAGAT |
| SEQ ID No: 7 | ATGACATCGTAGGTCAGCTGCGACCAGACAG |
| | AT |
| SEQ ID No: 8 | ATGACATCGTAGGTCAGCTGCGACCAGAGTA |
| | CTGCACGTAGAT |
| SEQ ID No: 9 | TCTGCATGCCGTTGCAGTACGCTACGTGCAG |
| | G |
| SEQ ID No: 10 | TCGTACCAGTGCATGCCGTTGCAGTACGCTA |
| | CGTGCAGG |
| SEQ ID No: 11 | TCGTACCAGGCTGACTGCGTTCGATACCACT |
| | GCTTGG |
| SEQ ID No: 12 | TCGTCGCAGCTGACCTACGATGTCAT |
| SEQ ID No: 13 | TCTGCAGTACTCTGGTCGCAGCTGACCTACG |
| | ATGTCAT |
| SEQ ID No: 14 | TCGTCGCAGCTGACCTACGATGTCATGG |
| SEQ ID No: 15 | TCTGCAGTACTCTGGTCGCAGCTGACCTACG |
| | ATGTCATGG |
| SEQ ID No: 16 | TCTGTCTGGTCGCAGCTGACCTACGATGTCA |
| | TGG |
| SEQ ID No: 17 | TCTACGTGCAGTACTCTGGTCGCAGCTGACC |
| | TACGATGTCATGG |
| SEQ ID No: 18 | ATCGTCGCAGCTGACCTACGATGTCATGG |
| | (36xC) |
| SEQ ID No: 19 | ATCTGCAGTACTCTGGTCGCAGCTGACCTAC |
| | GATGTCAT |

TABLE 2
Adapter sequences from Table 1 post-deamination.

| SEQ ID No. | Sequence |
|---|---|
| SEQ ID No: 20 | TGUAUGTAGUGTAUTGUAAUGGUATGUAGAT |
| SEQ ID No: 21 | TGUAUGTAGUGTAUTGUAAUGGUATGUACTGGTACG |
| | AT |
| SEQ ID No: 22 | AAGUAGTGGTATUGAAUGUAGTUAGUGAT |
| SEQ ID No: 23 | AAGUAGTGGTATUGAAUGUAGTUAGUCTGGTACGAT |
| SEQ ID No: 24 | ATGAUATUGTAGGTUAGUTGUGAUGAT |
| SEQ ID No: 25 | ATGAUATUGTAGGTUAGUTGUGAUCAGAGTACTGCA |
| | GAT |
| SEQ ID No: 26 | ATGAUATUGTAGGTUAGUTGUGAUCAGACAGAT |
| SEQ ID No: 27 | ATGAUATUGTAGGTUAGUTGUGAUCAGAGTACTGCA |
| | CGTAGAT |
| SEQ ID No: 28 | TUTGUATGUUGTTGUAGTAUGUTAUGTGUAGG |
| SEQ ID No: 29 | TCGTACCAGTGUATGUUGTTGUAGTAUGUTAUGTGU |
| | AGG |
| SEQ ID No: 30 | TCGTACCAGGUTGAUTGUGTTUGATAUUAUTGUTTG |
| | G |
| SEQ ID No: 31 | TUGTUGUAGUTGAUUTAUGATGTUAT |
| SEQ ID No: 32 | TCTGCAGTACTCTGGTUGUAGUTGAUUTAUGATGTU |
| | AT |
| SEQ ID No: 33 | TUGTUGUAGUTGAUUTAUGATGTUATGG |
| SEQ ID No: 34 | TCTGCAGTACTCTGGTUGUAGUTGAUUTAUGATGTU |
| | ATGG |
| SEQ ID No: 35 | TCTGTCTGGTUGUAGUTGAUUTAUGATGTUATGG |
| SEQ ID No: 36 | TCTACGTGCAGTACTCTGGTUGUAGUTGAUUTAUGA |
| | TGTUATGG |
| SEQ ID No: 37 | ATUGTUGUAGUTGAUUTAUGATGTUATGG(36xU) |
| SEQ ID No: 38 | ATCTGCAGTACTCTGGTUGUAGUTGAUUTAUGATGT |
| | UAT |

TABLE 3
Primer sequences for post conversion
adapter sequences

| SEQ ID No. | Sequence |
|---|---|
| SEQ ID No: 39 | ATGATATTGTAGGTTAGTTGTGAT |
| SEQ ID No: 40 | ATGATATTGTAGGTTAGTTGTGATG |
| SEQ ID No: 41 | ATAACATCATAAATCAACTACAAC |
| SEQ ID No: 42 | ATGTAGTGTATTGTAATGGTATGT |
| SEQ ID No: 43 | ACATAACATACTACAACAACATAC |
| SEQ ID No: 44 | TAGTGGTATTGAATGTAGTTAGT |
| SEQ ID No: 45 | CAATAATATCAAACACAATCAAC |

### Alternative Library Adapters

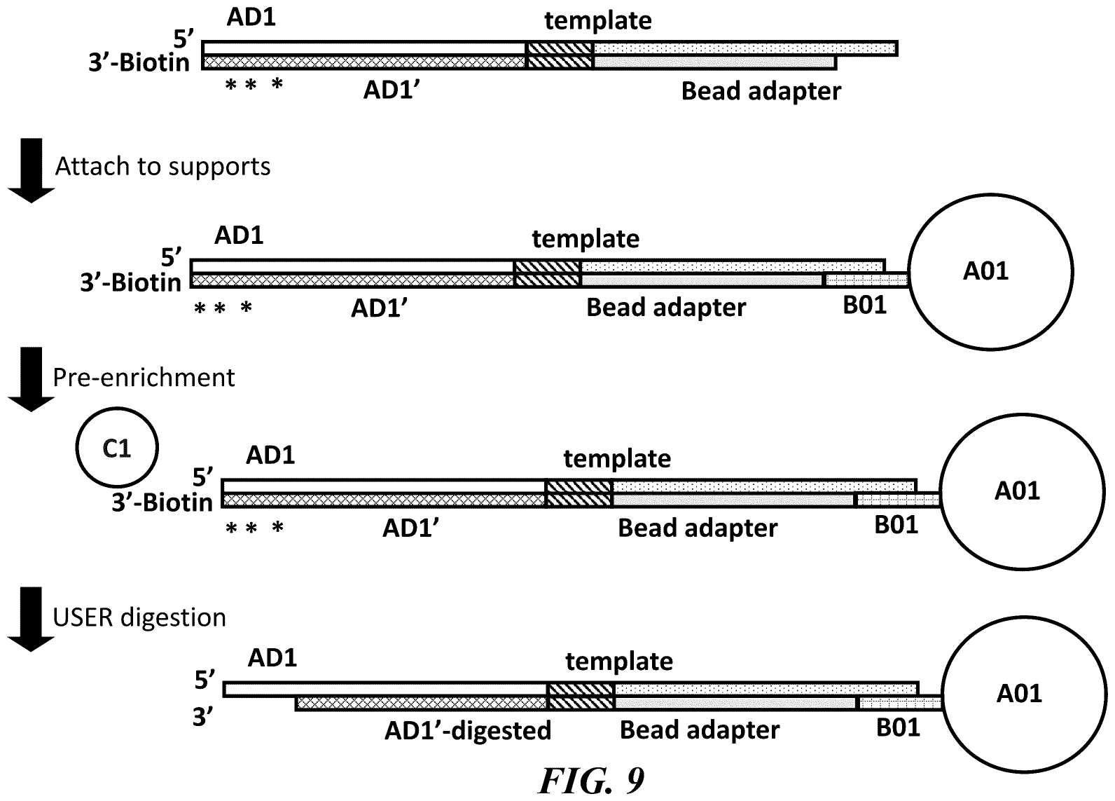

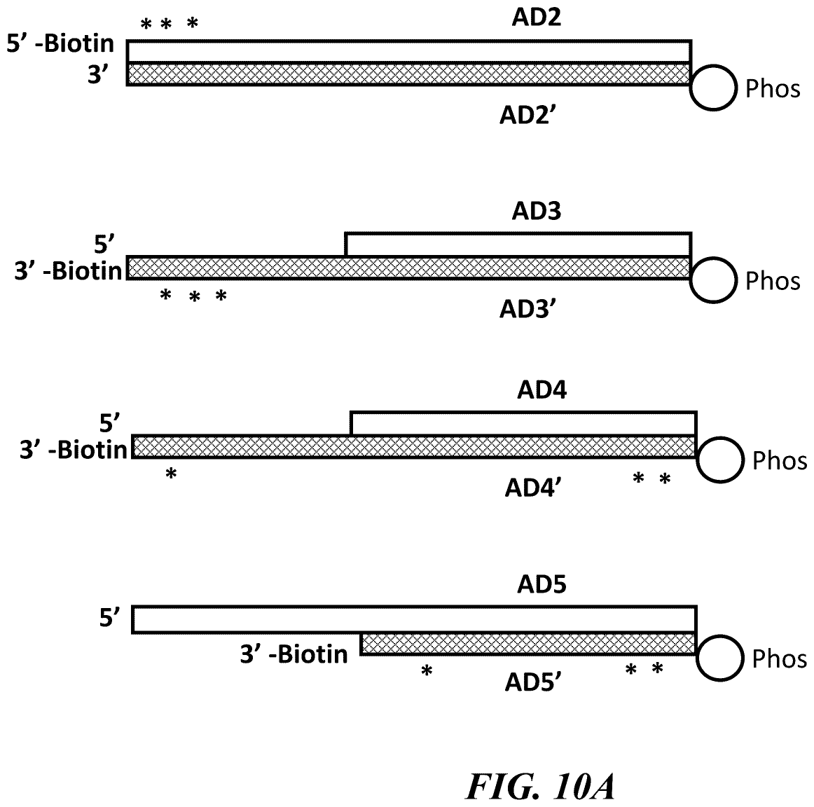

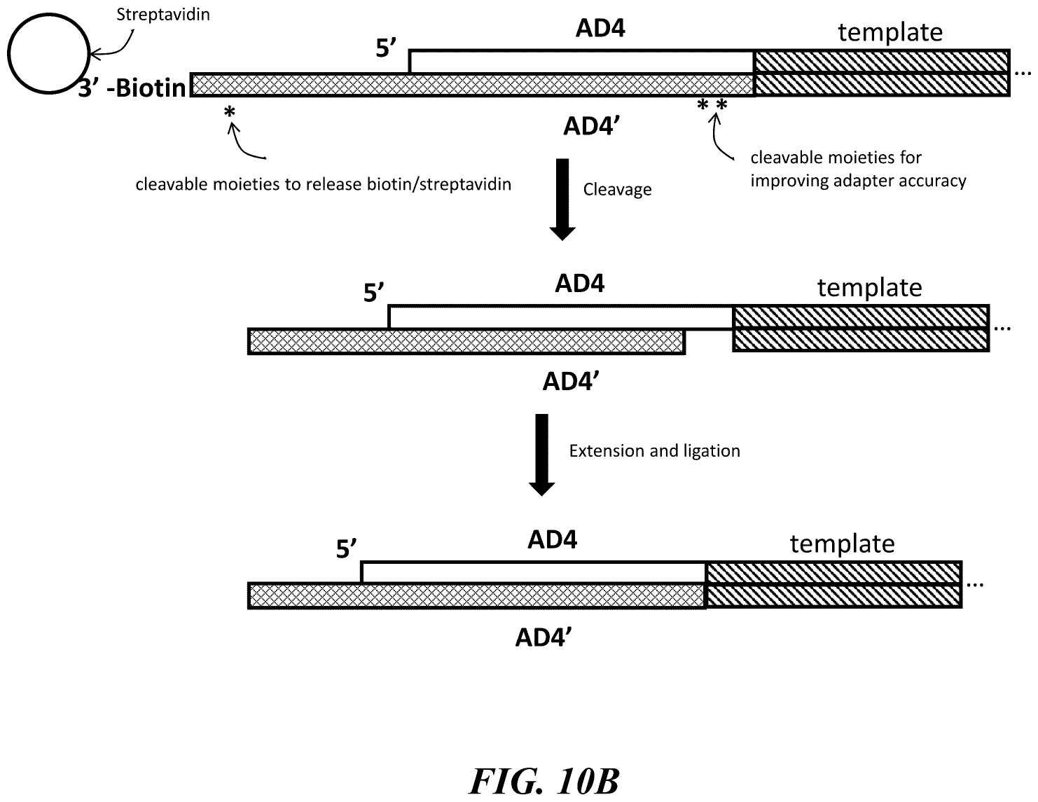

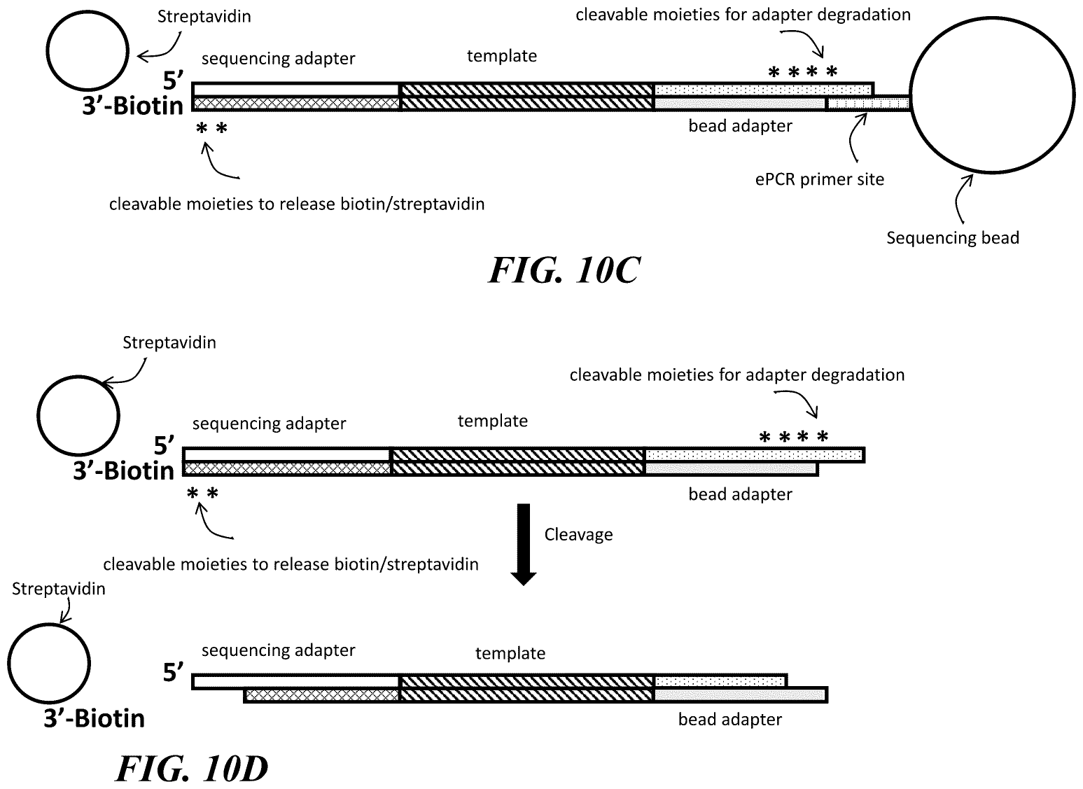

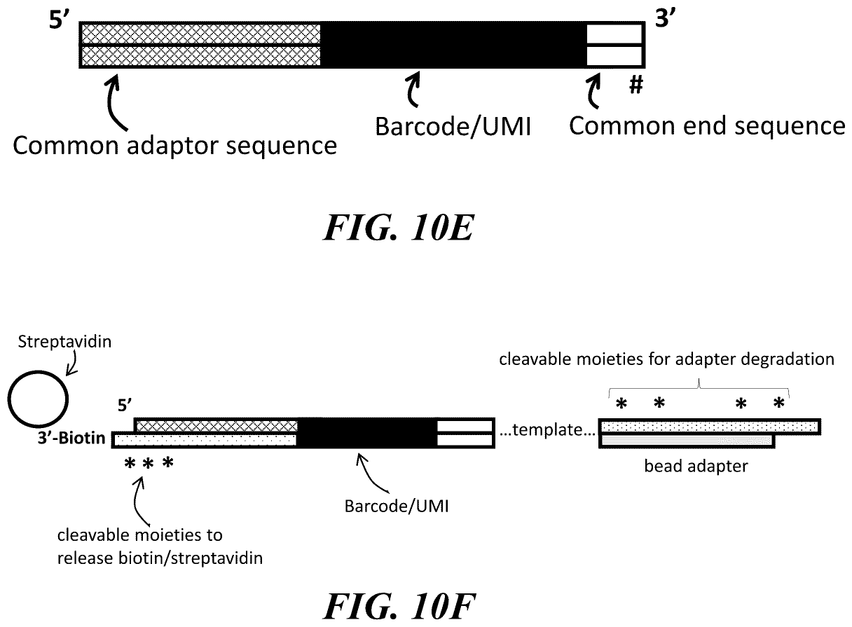

The devices, systems, methods, compositions, and kits provided herein may allow for the efficient and accurate preparation of sequencing libraries. An example scheme is illustrated in FIG. 9 and example adapter molecule constructs are shown in FIG. 10A. Unlike the single adapter species described above with respect to methylation adapters, library preparation with these alternative adapters will use two different adapter species. As illustrated in FIG. 9, a first adapter species (e.g., AD1/AD1′) is coupled to a capture tag (e.g., biotin) at a first end and a double-stranded template at the opposite end; and a second adapter species (e.g., Bead adapter) is coupled to a support (e.g., a sequencing bead) for downstream processing. The capture tag-coupled adapters may be modified to improve the accuracy of library construction, as described below.

Adapters with Higher Accuracy Sequences

Oligos, for example for library preparation, are synthesized in the 3′ to 5′ direction. As with all chemical reactions, there is a potential for error during synthesis; specifically not all of the oligos produced will be the same length (e.g., the chemical synthesis of oligos will not proceed to completion with 100% efficiency). Hence, there is a non-zero probability that intended 5′ oligo modifications (e.g., an affinity tag) will be missing from a subset of provided adapter oligos. This is not detrimental in the case of many oligo uses (e.g., PCR). However, with adapter molecules, any library molecules lacking an affinity tag will be wasted (e.g., without a biotin tag—which is typically a 5′ modification—they will be lost during pre-enrichment as they will be incapable of being captured by streptavidin). Since there is typically a limited amount of template molecules in a sample there is a need to improve the efficiency of library production and usage.

Adapters such as AD1/AD1′, which are illustrated in FIG. 9, comprise biotin disposed at a 3′ end. Thus, given the chemical process for oligo synthesis, all or substantially all such adapters should include these 3′ modifications. These adapter species may be used in library preparation as detailed in FIG. 9. Referring to FIG. 9, library molecules comprising an adapter AD1/AD1′ coupled to a template molecule (e.g., ligated to the template molecule) are provided. The bottom strand of the adapter, AD1′, is coupled to biotin at a 3′ end and comprises a plurality of cleavable moieties (*) disposed at the 3′ end. The bottom strand of the adapter, AD1′ may further comprise a 5′ phosphate for ligation. The library molecules are attached to supports (e.g., bead A01). After attachment of library molecules to supports, pre-enrichment is performed. In some cases, the pre-enrichment comprises addition of streptavidin (Cl), where streptavidin molecules couple to biotin molecules and serve to capture biotin-bound library molecules. In some cases, the cleavable moieties comprise uracils. In some such cases, uracil-specific excision reagent (USER) enzyme digestion is performed after pre-enrichment, thereby removing 3′ biotin from the AD1′ strand of the adapter, and concurrently any biotin-bound streptavidin molecules. In some cases, the cleavable moieties are not uracils, and an alternative to USER digestion is performed as appropriate after pre-enrichment. After USER digestion (or appropriate equivalent), emulsification PCR may be performed (or appropriate alternative).

In FIG. 10A, Adapter AD2/AD2′ serves as an example adapter species coupled to an affinity tag at a 5′ end (e.g., a biotin coupled to the 5′ end of strand AD2), to illustrate the contrast with adapter species AD1/AD1′. AD2/AD2′ further comprises a plurality of cleavable moieties disposed at the 5′ end of strand AD2 (e.g., for release of the affinity tag, as illustrated in FIG. 9). In some instances, AD2′ further comprises a 5′phosphate for ligation to a library molecule. The two strands of an AD2/AD2′ species of adapter are typically approximately the same length. Example sequences for AD2 and AD2′ sequences are listed in Table 4.

TABLE 4
Adapter sequences with 5′ biotin
(SEQ ID No: 46) and 5′ phosphate
(SEQ ID No: 47).

| SEQ ID No. | Sequence |
|---|---|
| SEQ ID No: 46 | UCCAUCUCATCCCTGCGTGTCTCCGACTGCACAC |
| | ATCCTGCATGTGAT |
| SEQ ID No: 47 | TCACATGCAGGATGTGTGCAGTCGGAGACACGCA |
| | GGGATGAGATGG |

FIG. 10A illustrates additional exemplary species of adapter molecules (e.g., AD3/AD3′, AD4/AD4′, and AD5/AD5′) that can be used in accord with the scheme in FIG. 9.

Additional adapter AD3/AD3′ is coupled to an affinity tag at a 3′ end (e.g., a biotin coupled to the 3′ end of strand AD3′) and further comprises a plurality of cleavable moieties disposed at the 3′ end of strand AD3′. In addition, AD3′ further comprises a 5′ phosphate for ligation to a library molecule. In some cases, strand AD3 is shorter in length than strand AD3′. For example, in some cases strand AD3 is about 90%, 80%, 70%, 60%, 50% or 40% of the length of strand AD3′. SEQ ID Nos: 49 and 48 are examples of AD3 and AD3′ sequences, respectively. Additional examples of AD3 sequences are SEQ ID Nos: 50, 51, 54, and 56, while SEQ ID No: 52, 53, and 55 are additional examples of AD3′ sequences.

Additional adapter AD4/AD4′ is coupled to an affinity tag at a 3′ end (e.g., a biotin coupled to the 3′ end of strand AD4′) and further comprises one or more cleavable moieties disposed at the 3′ end of strand AD4′ and one or more cleavable moieties disposed at the 5′ end of strand AD4′. In some cases, strand AD4 is shorter in length than strand AD4′. For example, in some cases strand AD4 is about 90%, 80%, 70%, 60%, 50% or 40% of the length of strand AD4′. SEQ ID Nos: 58 and 57 are examples of AD4 and AD4′ sequences, respectively. FIG. 10B illustrates the result of cleavage of the multiple cleavable moieties on adapter AD4/AD4′.

Additional adapter AD5/AD5′ is coupled to an affinity tag at a 3′ end (e.g., a biotin coupled to the 3′ end of strand AD5′) and further comprises one or more cleavable moieties disposed at the 3′ end of strand AD5′ and one or more cleavable moieties disposed at the 5′ end of strand AD5′. In some cases, strand AD5′ is shorter in length than strand AD5. For example, in some cases strand AD5′ is about 90%, 80%, 70%, 60%, 50% or 40% of the length of strand AD5. SEQ ID Nos: 50 and 59 are examples of AD5 and AD5′ sequences, respectively.

As can be seen in FIG. 10A, cleavable moieties in adapters AD3/AD3′, AD4/AD4′, and AD5/AD5′ are disposed 3′ in the strand coupled to the affinity tag (e.g., biotin). At least one cleavable moiety must be located near an end of the adapter coupled to an affinity tag; when this cleavable moiety is cleaved, the adapter is released from the biotin (e.g., for pre-enrichment of adapter-template complexes as described with respect to FIG. 9).

Adapters AD4/AD4′ and AD5/AD5′ further comprise two or more cleavable moieties disposed 5′ on the strand coupled to the affinity tag (e.g., near the end of the adapter comprising a phosphate). These 5′ cleavable moieties serve a separate purpose from 3′ cleavable moieties: specifically, 5′ cleavable moieties increase the accuracy of adapter sequences (i.e., the 5′ part of adapter strands AD4′ and AD5′). As discussed above, oligos are synthesized in the 3′ to 5′ and are thus subject to a higher proportion of errors at the 5′ ends. After cleavage of the 5′ cleavable moieties in adapters AD4/AD4′ and AD5/AD5′, an additional extension and ligation reaction is performed (see e.g., FIG. 10B). The additional extension and ligation reaction thus increases the probability that the 5′ end of the adapter bottom strand will be the correct sequence (i.e., because the extension is determined by the 3′ end of the respective adapter top strand).

In some embodiments, the cleavable moiety(ies) comprises uracil, ribonucleotide, spacer(s), or methylated nucleotide(s). In some embodiments, the spacer is a dSpacer or a C3 spacer. In some embodiments, cleaving the cleavable moiety(ies) comprises using APE1 enzyme to cleave the spacer(s). In some embodiments, the cleavable moiety(ies) is a methylated nucleotide(s) and cleaving the cleavable moiety(ies) comprises using MspJI to cleave the methylated nucleotide(s). In some embodiments, the cleavable moiety(ies) is a uracil and wherein cleaving the cleavable moiety(ies) comprises using a uracil D glycosylase (UDG) to cleave the uracil. In some embodiments, the cleavable moiety(ies) is a ribonucleotide(s) and cleaving the cleavable moiety(ies) comprises using a RNase to cleave the ribonucleotide(s). In some instances, each cleavable moiety in a respective strand of an adapter molecule is a same type (e.g., all uracils, all ribonucleotides, etc.).

In some instances, adapter sequences (e.g., bead-bound adapter strands) may further comprise a 3′ blocking group (e.g., to provide steric hindrance). In some instances, this 3′ blocking group may be one or more C3 spacers. In some instances, this 3′ blocking group is 1, 2, 3, 4, 5, 6, 7, 8, 9, or 10 C3 spacers.

Alternative adapter sequences for use as illustrated in FIG. 9 are provided in Table 5.

TABLE 5
Alternative adapter sequences with
3′ biotin and 5′ phosphate.

| SEQ ID No. | Sequence |
|---|---|
| SEQ ID No: 48 | TCACATGCAGGATGTGTGCAGTCGGAGACACGCA |
| | GGGAUGAGAUGGU |
| SEQ ID No: 49 | ATCTCATCCCTGCGTGTCTCCGACTGCAC |
| SEQ ID No: 50 | ATCTCATCCCTGCGTGTCTCCGACTGCACACATC |
| | CTGCATGTGAT |
| SEQ ID No: 51 | ATCTCATCCCTGCGTGTCTCCGACTGCACAGTTC |
| | ATCTGTGAT |
| SEQ ID No: 52 | TCACAGATGAACTGTGCAGTCGGAGACACGCAGG |
| | GAUGAGAUGGU |
| SEQ ID No: 53 | TCACATGCAGGATGTGTGCAGTCGGAGACACGCA |
| | GGGAUGAGAU |
| SEQ ID No: 54 | CCGACTGCACACATCCTGCATGTGAT |
| SEQ ID No: 55 | TCTGCATCTAGCCGTGCAGTCGGAGACACGCAGG |
| | GAUGAGAT |
| SEQ ID No: 56 | CCGACTGCACGGCTAGATGCAGAT |
| SEQ ID No: 57 | UCTGCTGAAUGCATCGTGCAGTCGGAGACACGCA |
| | GGGAUGAGAT |
| SEQ ID No: 58 | CCGACTGCACGATGCATTCAGCAGAT |
| SEQ ID No: 59 | TCACAUGCAGGATGTGUGCAGUCGG |

### Adapters for Decreased Polyclonality

Another issue that can be addressed with adapter modification is polyclonality. Polyclonality, where a single sequencing bead comprises a mixture of two or more templates (e.g., adapter-template molecules) for sequencing, is an undesirable result from ePCR e.g., when multiple distinct template molecules are present in a single reaction droplet. This can arise when free library molecules (e.g., library molecules that are not coupled to a sequencing bead, but which were captured by streptavidin during pre-enrichment) are present in a reaction mixture. The devices, systems, methods, compositions, and kits provided herein may allow for the production of sequencing libraries with decreased polyclonality.

By the addition of additional cleavable sites, free adapters (e.g., those not coupled to a support such as a sequencing bead) may be degraded (e.g., upon exposure to cleaving conditions such as USER enzyme). This degradation of free adapter sequences helps reduce the rate of polyclonality on sequencing beads by preventing unattached library molecules that do mistakenly enter a reaction mixture (e.g., oil droplets during ePCR) from hybridizing to beads and being subsequently amplified. These additional cleavable sites are distinct from the one or more cleavable sites that release adapters from streptavidin/biotin complexes.

Table 6 lists adapter sequences for decreased polyclonality. SEQ ID Nos: 62 and 63 are adapter sequences with 3′ biotin (e.g., such as described above with regards to AD3/AD3′ adapters). SEQ ID Nos: 60 and 61 are bead adapter sequences, as illustrated in FIG. 10C. SEQ ID No: 60 comprises multiple cleavage sites (e.g., uracils) disposed 5′. Upon exposure to USER, cleaving these cleavable moieties, degraded free adapter molecules will not be able to amplify in any downstream processing steps (see e.g., FIG. 10D and Example 2).

TABLE 6
Alternative adapter sequences for
decreased polyclonality.

| SEQ ID No. | Sequence |
|---|---|
| SEQ ID No: 60 | AUCACCGACUGCCCAUAGAGAGCTGAGACUGCCA |
| | AGGCACACAGG |
| SEQ ID No: 61 | CTCTCTATGGGCAGTCGGTGAT |
| SEQ ID No: 62 | ATCTCATCCCTGCGTGTCTCCGACTGCACGAT |
| SEQ ID No: 63 | TCGTGCAGTCGGAGACACGCAGGGAUGAGAUGGU |
| SEQ ID No: 64 | ATCACCGACUGCCCAUAGAGAGCTGAGACUGCCA |
| | AGGCACACAGG |
| SEQ ID No: 65 | ATCACCGACUGCCCAUAGAGAGTCTGAGACUGCC |
| | AAGGCACACAG*G |
| SEQ ID No: 66 | ATCACCrGACTrGCCCATAGArGAGTCTGAGACT |
| | rGCCAAGGCACACAG*G |
| SEQ ID No: 67 | ATCACCrGACTGCCCATAGArGAGCTGAGACTrG |
| | CCAAGGCACACAG*G |
| SEQ ID No: 68 | ATCACCrGACTrGCCCATAGArGAGCTGAGACTr |
| | GCCAAGGCACACAG*G |
| SEQ ID No: 69 | AUCACCGACUGCCCAUAGAGAGCTGAGACUGCCA |
| | AGGCACACAGG |
| SEQ ID No: 70 | TCTrCAGrCTCTCTATGGGCAGTCGGTGAT |
| SEQ ID No: 71 | CCTrGTGTGCCTTGrGCArGTCTCArGCTCTCTA |
| | TGGGCAGTCGGTGAT |
| SEQ ID No: 72 | TCCTCTCTATGGGCAGTCGGTGAT |
| SEQ ID No: 73 | AGCTCTCTATGGGCAGTCGGTGAT |
| SEQ ID No: 74 | GACTCTGACGGTTCCGTGTGTCCCCTATCC |

SEQ ID Nos: 64, 65, 66, 67, 68, and 69, are alternative sequences to SEQ ID No: 60. SEQ ID Nos: 70, 71, 72, and 73 are alternative sequences to SEQ ID No: 61. Also in Table 6 are additional sequences for use in accordance with ePCR. For instance, SEQ ID No: 74 is an example ePCR primer site (as illustrated in FIG. 10C). Different combinations of the bead adapter sequences listed in Table 6 are possible. In Table 6, as elsewhere, * indicates a phosphorothioated base and ‘r’ indicates a ribonucleobase. Adapter sequences with one or more ribonucleobases may require RNase HII treatment prior to downstream processing.

### Adapters for PCR-Free Library Construction

In some instances, adapter molecules as described above may be used for the efficient preparation of PCR-free libraries (e.g., for sensitive sample preparations). An example set of PCR-free adapter and primer sequences are shown in Table 7, and FIG. 10F provides a block diagram of a PCR-free adapter-template complex. PCR-free library adapter sequences (e.g., bead-side adapters) may, in some instances, be combined with other sets of adapter sequences (e.g., sequencing adapters as illustrated in FIG. 10C) described herein. For instance, for a PCR-free construct the top strand of the biotin-coupled adapter may comprise SEQ ID No: 68, with modifications being made to the bottom strand of the biotin-coupled adapter.

TABLE 7
PCR-free library structures

| SEQ ID No. | Sequence |
|---|---|
| SEQ ID No: 75 | AUCACCGACUGCCCAUAGAGAGCT |
| | GAGACUGCCAAGGCA |
| SEQ ID No: 76 | CTCTCTATGGGCAGTCGGTGAT*T |

where * indicates a phosphorothioated base. In some instances, SEQ ID No: 76 does not comprise a phosphorothioated base.

Further example adapters that may be used as described herein are listed in Tables 8, 9, and 10. In Table 8 and Table 9, * indicates a phosphorothioated base. As can be seen in Table 8, each sequence comprises at least sequence ATCTCATCCCTGCGTGTCTCCGACTGCAC (SEQ ID No: 49) disposed at the 5′ end of the adapter sequence. Each sequence further comprises the sequence GAT disposed at the 3′ end of the adapter sequence, where the T is phosphorothioated. The intervening region in each sequence in Table 8 is variable and unique from other intervening regions in Table 8 (e.g., the intervening region is a barcode sequence). In Table 9, the adapter sequences from Table 8 have been modified to include two internal uracil residues (e.g., replacing two thymine residues). These internal uracils serve to prevent the amplification of free library molecules (e.g., as described with respect to FIG. 10D). In Table 10, in some instances, each sequence has a 5′ phosphate and a 3′ biotin-TEG tag.

TABLE 8
PCR-free adapters.

| SEQ ID No. | Sequence |
|---|---|
| SEQ ID No: 77 | ATCTCATCCCTGCGTGTCTCCGACTGCACAGCTCGAATGCGA*T |
| SEQ ID No: 78 | ATCTCATCCCTGCGTGTCTCCGACTGCACATGTGCAGCCATCGA*T |
| SEQ ID No: 79 | ATCTCATCCCTGCGTGTCTCCGACTGCACATCACACATGAATGA*T |
| SEQ ID No: 80 | ATCTCATCCCTGCGTGTCTCCGACTGCACTGTGTAGGCATGA*T |
| SEQ ID No: 81 | ATCTCATCCCTGCGTGTCTCCGACTGCACATGTATCCTCTGA*T |
| SEQ ID No: 82 | ATCTCATCCCTGCGTGTCTCCGACTGCACATATAGCCTATGA*T |
| SEQ ID No: 83 | ATCTCATCCCTGCGTGTCTCCGACTGCACGATTCATGCTCGA*T |
| SEQ ID No: 84 | ATCTCATCCCTGCGTGTCTCCGACTGCACACATCCTGCATGTGA*T |
| SEQ ID No: 85 | ATCTCATCCCTGCGTGTCTCCGACTGCACGCGCATCCTGCATGA*T |
| SEQ ID No: 86 | ATCTCATCCCTGCGTGTCTCCGACTGCACACTGCACGAATGA*T |
| SEQ ID No: 87 | ATCTCATCCCTGCGTGTCTCCGACTGCACACGCACTGCCAGA*T |
| SEQ ID No: 88 | ATCTCATCCCTGCGTGTCTCCGACTGCACTGCCATAGCACGA*T |
| SEQ ID No: 89 | ATCTCATCCCTGCGTGTCTCCGACTGCACAGTTGTGCTGTGA*T |
| SEQ ID No: 90 | ATCTCATCCCTGCGTGTCTCCGACTGCACATGGCGCGATCATGA*T |
| SEQ ID No: 91 | ATCTCATCCCTGCGTGTCTCCGACTGCACTTAGATATCATGA*T |
| SEQ ID No: 92 | ATCTCATCCCTGCGTGTCTCCGACTGCACATCCTGTGCGCATGA*T |
| SEQ ID No: 93 | ATCTCATCCCTGCGTGTCTCCGACTGCACACGTGGCACATGA*T |
| SEQ ID No: 94 | ATCTCATCCCTGCGTGTCTCCGACTGCACATGCGTCCTGTGA*T |
| SEQ ID No: 95 | ATCTCATCCCTGCGTGTCTCCGACTGCACAATGCTCTGCATAGA*T |
| SEQ ID No: 96 | ATCTCATCCCTGCGTGTCTCCGACTGCACTACATTGCACAGA*T |
| SEQ ID No: 97 | ATCTCATCCCTGCGTGTCTCCGACTGCACATAGCGAGCCAGA*T |
| SEQ ID No: 98 | ATCTCATCCCTGCGTGTCTCCGACTGCACGGAGTGCATGCATGA*T |
| SEQ ID No: 99 | ATCTCATCCCTGCGTGTCTCCGACTGCACATGGAGATACTGA*T |
| SEQ ID No: 100 | ATCTCATCCCTGCGTGTCTCCGACTGCACTGAGCCTGTCAGA*T |
| SEQ ID No: 101 | ATCTCATCCCTGCGTGTCTCCGACTGCACTCGAGATTGATGA*T |
| SEQ ID No: 102 | ATCTCATCCCTGCGTGTCTCCGACTGCACATCCATCTCATCTGA*T |
| SEQ ID No: 103 | ATCTCATCCCTGCGTGTCTCCGACTGCACACTGTCAGCCAGA*T |
| SEQ ID No: 104 | ATCTCATCCCTGCGTGTCTCCGACTGCACAGACTTGCTGCGA*T |
| SEQ ID No: 105 | ATCTCATCCCTGCGTGTCTCCGACTGCACATATAGCAGGAGA*T |
| SEQ ID No: 106 | ATCTCATCCCTGCGTGTCTCCGACTGCACATGTCTAAGATGA*T |
| SEQ ID No: 107 | ATCTCATCCCTGCGTGTCTCCGACTGCACATCGCAGCTGAATGA*T |
| SEQ ID No: 108 | ATCTCATCCCTGCGTGTCTCCGACTGCACTCTGTATTGCAGA*T |
| SEQ ID No: 109 | ATCTCATCCCTGCGTGTCTCCGACTGCACATCATCGATTCGA*T |
| SEQ ID No: 110 | ATCTCATCCCTGCGTGTCTCCGACTGCACTGTGAATATGCGA*T |
| SEQ ID No: 111 | ATCTCATCCCTGCGTGTCTCCGACTGCACTGAATGATCTCGA*T |
| SEQ ID No: 112 | ATCTCATCCCTGCGTGTCTCCGACTGCACAGAAGCTGCATGAGA*T |
| SEQ ID No: 113 | ATCTCATCCCTGCGTGTCTCCGACTGCACTAGATTGCGCAGA*T |
| SEQ ID No: 114 | ATCTCATCCCTGCGTGTCTCCGACTGCACGCATCCTCACAGA*T |
| SEQ ID No: 115 | ATCTCATCCCTGCGTGTCTCCGACTGCACACAACATATCAGA*T |
| SEQ ID No: 116 | ATCTCATCCCTGCGTGTCTCCGACTGCACATCCTGCATCGCAGA*T |
| SEQ ID No: 117 | ATCTCATCCCTGCGTGTCTCCGACTGCACACATCAGCTCAATGA*T |
| SEQ ID No: 118 | ATCTCATCCCTGCGTGTCTCCGACTGCACATGCATATAATGCGA*T |
| SEQ ID No: 119 | ATCTCATCCCTGCGTGTCTCCGACTGCACTCAATGCATCAGCGA*T |
| SEQ ID No: 120 | ATCTCATCCCTGCGTGTCTCCGACTGCACAATGTGTGCTAGA*T |
| SEQ ID No: 121 | ATCTCATCCCTGCGTGTCTCCGACTGCACTCAGCCTCATGCTGA*T |
| SEQ ID No: 122 | ATCTCATCCCTGCGTGTCTCCGACTGCACTCTCGCATGCAATGA*T |
| SEQ ID No: 123 | ATCTCATCCCTGCGTGTCTCCGACTGCACTAGCAGCCAGCGA*T |
| SEQ ID No: 124 | ATCTCATCCCTGCGTGTCTCCGACTGCACTGCCAGACTGTGA*T |
| SEQ ID No: 125 | ATCTCATCCCTGCGTGTCTCCGACTGCACGCACTGCTCCTGA*T |
| SEQ ID No: 126 | ATCTCATCCCTGCGTGTCTCCGACTGCACACATGGCAGCACAGA*T |
| SEQ ID No: 127 | ATCTCATCCCTGCGTGTCTCCGACTGCACACTTGCAGATCGA*T |
| SEQ ID No: 128 | ATCTCATCCCTGCGTGTCTCCGACTGCACTATGCCACAGCATGA*T |
| SEQ ID No: 129 | ATCTCATCCCTGCGTGTCTCCGACTGCACAACATCAGCATGAGA*T |
| SEQ ID No: 130 | ATCTCATCCCTGCGTGTCTCCGACTGCACTGCAGTGATTCATGA*T |
| SEQ ID No: 131 | ATCTCATCCCTGCGTGTCTCCGACTGCACAGCACCTGCATCAGA*T |
| SEQ ID No: 132 | ATCTCATCCCTGCGTGTCTCCGACTGCACTTATGCTATCAGA*T |
| SEQ ID No: 133 | ATCTCATCCCTGCGTGTCTCCGACTGCACAATGCGTGCTGCAGA*T |
| SEQ ID No: 134 | ATCTCATCCCTGCGTGTCTCCGACTGCACATCTCAGTGCAATGA*T |
| SEQ ID No: 135 | ATCTCATCCCTGCGTGTCTCCGACTGCACATGTGCTTCGCATGA*T |
| SEQ ID No: 136 | ATCTCATCCCTGCGTGTCTCCGACTGCACATGAGTGCAGCCAGA*T |
| SEQ ID No: 137 | ATCTCATCCCTGCGTGTCTCCGACTGCACTCAAGCATGTGCTGA*T |
| SEQ ID No: 138 | ATCTCATCCCTGCGTGTCTCCGACTGCACACAGTCAATGTGA*T |
| SEQ ID No: 139 | ATCTCATCCCTGCGTGTCTCCGACTGCACACACTTGATGCGA*T |
| SEQ ID No: 140 | ATCTCATCCCTGCGTGTCTCCGACTGCACATAGAGCCTCAGA*T |
| SEQ ID No: 141 | ATCTCATCCCTGCGTGTCTCCGACTGCACTTGTGTCATGAGA*T |
| SEQ ID No: 142 | ATCTCATCCCTGCGTGTCTCCGACTGCACAATGCACTCGAGA*T |
| SEQ ID No: 143 | ATCTCATCCCTGCGTGTCTCCGACTGCACTTATGAGCGCTGA*T |
| SEQ ID No: 144 | ATCTCATCCCTGCGTGTCTCCGACTGCACGTCATTGCACAGA*T |
| SEQ ID No: 145 | ATCTCATCCCTGCGTGTCTCCGACTGCACATCACTGCAACGA*T |
| SEQ ID No: 146 | ATCTCATCCCTGCGTGTCTCCGACTGCACTTGCATGCGATGCGA*T |
| SEQ ID No: 147 | ATCTCATCCCTGCGTGTCTCCGACTGCACGTGCGCAAGCAGA*T |
| SEQ ID No: 148 | ATCTCATCCCTGCGTGTCTCCGACTGCACTATCTCATAATGA*T |
| SEQ ID No: 149 | ATCTCATCCCTGCGTGTCTCCGACTGCACATGGCTATGCACTGA*T |
| SEQ ID No: 150 | ATCTCATCCCTGCGTGTCTCCGACTGCACTATGAATGAGCGA*T |
| SEQ ID No: 151 | ATCTCATCCCTGCGTGTCTCCGACTGCACTATGCACCATCGA*T |
| SEQ ID No: 152 | ATCTCATCCCTGCGTGTCTCCGACTGCACACAATGTGCGCGA*T |
| SEQ ID No: 153 | ATCTCATCCCTGCGTGTCTCCGACTGCACAATGACTATCTGA*T |
| SEQ ID No: 154 | ATCTCATCCCTGCGTGTCTCCGACTGCACAGCCGCTGCTCGA*T |
| SEQ ID No: 155 | ATCTCATCCCTGCGTGTCTCCGACTGCACGCGCGCAGAATGA*T |
| SEQ ID No: 156 | ATCTCATCCCTGCGTGTCTCCGACTGCACAGCCTCAGCGTGA*T |
| SEQ ID No: 157 | ATCTCATCCCTGCGTGTCTCCGACTGCACTTGATCATACAGA*T |
| SEQ ID No: 158 | ATCTCATCCCTGCGTGTCTCCGACTGCACTCTGCTGTGCAATGA*T |
| SEQ ID No: 159 | ATCTCATCCCTGCGTGTCTCCGACTGCACAGTGTATTGCTGA*T |
| SEQ ID No: 160 | ATCTCATCCCTGCGTGTCTCCGACTGCACTGTGCATCTGCCTGA*T |
| SEQ ID No: 161 | ATCTCATCCCTGCGTGTCTCCGACTGCACTCTATGTTGCTGA*T |
| SEQ ID No: 162 | ATCTCATCCCTGCGTGTCTCCGACTGCACAACTATCTGCAGA*T |
| SEQ ID No: 163 | ATCTCATCCCTGCGTGTCTCCGACTGCACAGATCTCATGAATGA*T |
| SEQ ID No: 164 | ATCTCATCCCTGCGTGTCTCCGACTGCACTATCATCCAGTGA*T |
| SEQ ID No: 165 | ATCTCATCCCTGCGTGTCTCCGACTGCACTGCTACAAGCAGA*T |
| SEQ ID No: 166 | ATCTCATCCCTGCGTGTCTCCGACTGCACAATATGCACGTGA*T |
| SEQ ID No: 167 | ATCTCATCCCTGCGTGTCTCCGACTGCACACTTCTGCGATGA*T |
| SEQ ID No: 168 | ATCTCATCCCTGCGTGTCTCCGACTGCACAAGCATATCTAGA*T |
| SEQ ID No: 169 | ATCTCATCCCTGCGTGTCTCCGACTGCACAATGACAGCTCGA*T |
| SEQ ID No: 170 | ATCTCATCCCTGCGTGTCTCCGACTGCACTGATGAGCTTGATGA*T |
| SEQ ID No: 171 | ATCTCATCCCTGCGTGTCTCCGACTGCACATATGACCTGAGA*T |
| SEQ ID No: 172 | ATCTCATCCCTGCGTGTCTCCGACTGCACAGCCGATATCAGA*T |
| SEQ ID No: 173 | ATCTCATCCCTGCGTGTCTCCGACTGCACAGTCAGTTGCAGA*T |
| SEQ ID No: 174 | ATCTCATCCCTGCGTGTCTCCGACTGCACAACAGATGTATGA*T |
| SEQ ID No: 175 | ATCTCATCCCTGCGTGTCTCCGACTGCACTCATCTGCGCAATGA*T |
| SEQ ID No: 176 | ATCTCATCCCTGCGTGTCTCCGACTGCACGGATCATGCGTGA*T |
| SEQ ID No: 177 | ATCTCATCCCTGCGTGTCTCCGACTGCACTATTGCATGCTCTGA*T |
| SEQ ID No: 178 | ATCTCATCCCTGCGTGTCTCCGACTGCACTACCTGATGCAGA*T |
| SEQ ID No: 179 | ATCTCATCCCTGCGTGTCTCCGACTGCACATATAATCACAGA*T |
| SEQ ID No: 180 | ATCTCATCCCTGCGTGTCTCCGACTGCACATGCAGCGCTAATGA*T |
| SEQ ID No: 181 | ATCTCATCCCTGCGTGTCTCCGACTGCACATGGCGCAGTGCTGA*T |
| SEQ ID No: 182 | ATCTCATCCCTGCGTGTCTCCGACTGCACTGAGAATGTGTGA*T |
| SEQ ID No: 183 | ATCTCATCCCTGCGTGTCTCCGACTGCACATGCATGGTACGA*T |
| SEQ ID No: 184 | ATCTCATCCCTGCGTGTCTCCGACTGCACATGCATGCGAGGAGA*T |
| SEQ ID No: 185 | ATCTCATCCCTGCGTGTCTCCGACTGCACAATCTGCATACGA*T |
| SEQ ID No: 186 | ATCTCATCCCTGCGTGTCTCCGACTGCACAGCATGAGCGCCAGA*T |
| SEQ ID No: 187 | ATCTCATCCCTGCGTGTCTCCGACTGCACTTATCTGATCTGA*T |
| SEQ ID No: 188 | ATCTCATCCCTGCGTGTCTCCGACTGCACATCCAGCGCATGTGA*T |
| SEQ ID No: 189 | ATCTCATCCCTGCGTGTCTCCGACTGCACAGTTCATCTGTGA*T |
| SEQ ID No: 190 | ATCTCATCCCTGCGTGTCTCCGACTGCACAACATACATCAGA*T |
| SEQ ID No: 191 | ATCTCATCCCTGCGTGTCTCCGACTGCACGGCTAGATGCAGA*T |
| SEQ ID No: 192 | ATCTCATCCCTGCGTGTCTCCGACTGCACAGTTATGTGCTGA*T |
| SEQ ID No: 193 | ATCTCATCCCTGCGTGTCTCCGACTGCACTGCCGAGCAGCATGA*T |
| SEQ ID No: 194 | ATCTCATCCCTGCGTGTCTCCGACTGCACTGCCTCAGATCATGA*T |
| SEQ ID No: 195 | ATCTCATCCCTGCGTGTCTCCGACTGCACTGATCAGTGGCGA*T |
| SEQ ID No: 196 | ATCTCATCCCTGCGTGTCTCCGACTGCACTGCTGCGGAGCATGA*T |
| SEQ ID No: 197 | ATCTCATCCCTGCGTGTCTCCGACTGCACAGTGCTGTGGCATGA*T |
| SEQ ID No: 198 | ATCTCATCCCTGCGTGTCTCCGACTGCACACATATGGCATATGA*T |
| SEQ ID No: 199 | ATCTCATCCCTGCGTGTCTCCGACTGCACAGATCGCCACAGA*T |
| SEQ ID No: 200 | ATCTCATCCCTGCGTGTCTCCGACTGCACACGGCTCAGATGA*T |
| SEQ ID No: 201 | ATCTCATCCCTGCGTGTCTCCGACTGCACTTGATGATAGTGA*T |
| SEQ ID No: 202 | ATCTCATCCCTGCGTGTCTCCGACTGCACAACTGCACATAGA*T |
| SEQ ID No: 203 | ATCTCATCCCTGCGTGTCTCCGACTGCACGCATACATCCTGA*T |
| SEQ ID No: 204 | ATCTCATCCCTGCGTGTCTCCGACTGCACATCTCTGGCTGCAGA*T |
| SEQ ID No: 205 | ATCTCATCCCTGCGTGTCTCCGACTGCACATCTGGTGCATGTGA*T |
| SEQ ID No: 206 | ATCTCATCCCTGCGTGTCTCCGACTGCACAGCAGCTTCGCGA*T |
| SEQ ID No: 207 | ATCTCATCCCTGCGTGTCTCCGACTGCACGCATATGGCAGCAGA*T |
| SEQ ID No: 208 | ATCTCATCCCTGCGTGTCTCCGACTGCACACAGCACCGCTGA*T |
| SEQ ID No: 209 | ATCTCATCCCTGCGTGTCTCCGACTGCACTAGCATCTGGTGA*T |
| SEQ ID No: 210 | ATCTCATCCCTGCGTGTCTCCGACTGCACTTCAGCATACAGA*T |
| SEQ ID No: 211 | ATCTCATCCCTGCGTGTCTCCGACTGCACATGCAGATGGCGAGA*T |
| SEQ ID No: 212 | ATCTCATCCCTGCGTGTCTCCGACTGCACAGATCTATCCAGA*T |
| SEQ ID No: 213 | ATCTCATCCCTGCGTGTCTCCGACTGCACTTCATGCATCTCAGA*T |
| SEQ ID No: 214 | ATCTCATCCCTGCGTGTCTCCGACTGCACATGCAAGTGTGATGA*T |
| SEQ ID No: 215 | ATCTCATCCCTGCGTGTCTCCGACTGCACTGTTCGCTGCAGA*T |
| SEQ ID No: 216 | ATCTCATCCCTGCGTGTCTCCGACTGCACTCAGATCCTGCATGA*T |
| SEQ ID No: 217 | ATCTCATCCCTGCGTGTCTCCGACTGCACACACAGATAATGA*T |
| SEQ ID No: 218 | ATCTCATCCCTGCGTGTCTCCGACTGCACGATGCTCTGGCGA*T |
| SEQ ID No: 219 | ATCTCATCCCTGCGTGTCTCCGACTGCACAGATCCATCATCTGA*T |
| SEQ ID No: 220 | ATCTCATCCCTGCGTGTCTCCGACTGCACTGCACCATCATATGA*T |
| SEQ ID No: 221 | ATCTCATCCCTGCGTGTCTCCGACTGCACATGCTATAGCAATGA*T |
| SEQ ID No: 222 | ATCTCATCCCTGCGTGTCTCCGACTGCACATCTGCACATGGCGA*T |
| SEQ ID No: 223 | ATCTCATCCCTGCGTGTCTCCGACTGCACGCTGCATGAAGATGA*T |
| SEQ ID No: 224 | ATCTCATCCCTGCGTGTCTCCGACTGCACTATTCAGATGCATGA*T |
| SEQ ID No: 225 | ATCTCATCCCTGCGTGTCTCCGACTGCACTCGCGCAATGCGA*T |
| SEQ ID No: 226 | ATCTCATCCCTGCGTGTCTCCGACTGCACGCGCAGATGGCATGA*T |
| SEQ ID No: 227 | ATCTCATCCCTGCGTGTCTCCGACTGCACAGTGAATCATCGA*T |
| SEQ ID No: 228 | ATCTCATCCCTGCGTGTCTCCGACTGCACATGCAACACTAGA*T |
| SEQ ID No: 229 | ATCTCATCCCTGCGTGTCTCCGACTGCACTATTATGATATGA*T |
| SEQ ID No: 230 | ATCTCATCCCTGCGTGTCTCCGACTGCACTCAGAAGCATCGA*T |
| SEQ ID No: 231 | ATCTCATCCCTGCGTGTCTCCGACTGCACTTCATATCTGAGA*T |
| SEQ ID No: 232 | ATCTCATCCCTGCGTGTCTCCGACTGCACAATAGCTATGAGA*T |
| SEQ ID No: 233 | ATCTCATCCCTGCGTGTCTCCGACTGCACATGGATAGCTGCAGA*T |
| SEQ ID No: 234 | ATCTCATCCCTGCGTGTCTCCGACTGCACATAATATCTGCGA*T |
| SEQ ID No: 235 | ATCTCATCCCTGCGTGTCTCCGACTGCACGCATGCATCCTGAGA*T |
| SEQ ID No: 236 | ATCTCATCCCTGCGTGTCTCCGACTGCACAGCACAGAGGAGA*T |
| SEQ ID No: 237 | ATCTCATCCCTGCGTGTCTCCGACTGCACAACTGCGCTCTGA*T |
| SEQ ID No: 238 | ATCTCATCCCTGCGTGTCTCCGACTGCACACAAGATGACAGA*T |
| SEQ ID No: 239 | ATCTCATCCCTGCGTGTCTCCGACTGCACAGATGACATTAGA*T |
| SEQ ID No: 240 | ATCTCATCCCTGCGTGTCTCCGACTGCACATGGCTCACATATGA*T |
| SEQ ID No: 241 | ATCTCATCCCTGCGTGTCTCCGACTGCACAAGAGATGCATCTGA*T |
| SEQ ID No: 242 | ATCTCATCCCTGCGTGTCTCCGACTGCACATGACCGCTGTGA*T |
| SEQ ID No: 243 | ATCTCATCCCTGCGTGTCTCCGACTGCACGCATCTGGCGCGA*T |
| SEQ ID No: 244 | ATCTCATCCCTGCGTGTCTCCGACTGCACTTAGCTGTGATGA*T |
| SEQ ID No: 245 | ATCTCATCCCTGCGTGTCTCCGACTGCACTCGCAATAGATGA*T |
| SEQ ID No: 246 | ATCTCATCCCTGCGTGTCTCCGACTGCACACTCTCTCAATGA*T |
| SEQ ID No: 247 | ATCTCATCCCTGCGTGTCTCCGACTGCACACTGCCTGATGATGA*T |
| SEQ ID No: 248 | ATCTCATCCCTGCGTGTCTCCGACTGCACTCTATTCATATGA*T |
| SEQ ID No: 249 | ATCTCATCCCTGCGTGTCTCCGACTGCACTTGAGCGCACTGA*T |
| SEQ ID No: 250 | ATCTCATCCCTGCGTGTCTCCGACTGCACGCTGATCAGGCGA*T |
| SEQ ID No: 251 | ATCTCATCCCTGCGTGTCTCCGACTGCACGCTCTGTTGATGA*T |
| SEQ ID No: 252 | ATCTCATCCCTGCGTGTCTCCGACTGCACTCAACATATCTGA*T |
| SEQ ID No: 253 | ATCTCATCCCTGCGTGTCTCCGACTGCACATGCGGCGCAGCAGA*T |
| SEQ ID No: 254 | ATCTCATCCCTGCGTGTCTCCGACTGCACTTGCTGTGCGCGA*T |
| SEQ ID No: 255 | ATCTCATCCCTGCGTGTCTCCGACTGCACATAGCGTTCATGA*T |
| SEQ ID No: 256 | ATCTCATCCCTGCGTGTCTCCGACTGCACGCACATAGCCTGA*T |
| SEQ ID No: 257 | ATCTCATCCCTGCGTGTCTCCGACTGCACTTCTCATGTCAGA*T |
| SEQ ID No: 258 | ATCTCATCCCTGCGTGTCTCCGACTGCACAGCCATCTGCTGAGA*T |
| SEQ ID No: 259 | ATCTCATCCCTGCGTGTCTCCGACTGCACACTCAGCGCCAGA*T |
| SEQ ID No: 260 | ATCTCATCCCTGCGTGTCTCCGACTGCACAACATCTGATAGA*T |
| SEQ ID No: 261 | ATCTCATCCCTGCGTGTCTCCGACTGCACTGCTCCGCATCGA*T |
| SEQ ID No: 262 | ATCTCATCCCTGCGTGTCTCCGACTGCACTATCGCTTGATGA*T |
| SEQ ID No: 263 | ATCTCATCCCTGCGTGTCTCCGACTGCACTCTTCTCAGCAGA*T |
| SEQ ID No: 264 | ATCTCATCCCTGCGTGTCTCCGACTGCACATGGTGTGCACGA*T |
| SEQ ID No: 265 | ATCTCATCCCTGCGTGTCTCCGACTGCACATATCATCTTAGA*T |
| SEQ ID No: 266 | ATCTCATCCCTGCGTGTCTCCGACTGCACGGTGCTGCTATGA*T |
| SEQ ID No: 267 | ATCTCATCCCTGCGTGTCTCCGACTGCACTGTCAGCCTATGA*T |
| SEQ ID No: 268 | ATCTCATCCCTGCGTGTCTCCGACTGCACATGATCACACAATGA*T |

TABLE 9
PCR-free adapters with internal uracils

| SEQ ID No. | Sequence |
|---|---|
| SEQ ID No: 269 | ATCTCATCCCTGCGTGTC/ideoxyU/CCGAC/ideoxyU/GCACAGCTCGAATGCGA |
| | *T |
| SEQ ID No: 270 | ATCTCATCCCTGCGTGTC/ideoxyU/CCGAC/ideoxyU/GCACATGTGCAGCCATC |
| | GA*T |
| SEQ ID No: 271 | ATCTCATCCCTGCGTGTC/ideoxyU/CCGAC/ideoxyU/GCACATCACACATGAAT |
| | GA*T |
| SEQ ID No: 272 | ATCTCATCCCTGCGTGTC/ideoxyU/CCGAC/ideoxyU/GCACTGTGTAGGCATGA |
| | *T |
| SEQ ID No: 273 | ATCTCATCCCTGCGTGTC/ideoxyU/CCGAC/ideoxyU/GCACATGTATCCTCTGA |
| | *T |
| SEQ ID No: 274 | ATCTCATCCCTGCGTGTC/ideoxyU/CCGAC/ideoxyU/GCACATATAGCCTATGA |
| | *T |
| SEQ ID No: 275 | ATCTCATCCCTGCGTGTC/ideoxyU/CCGAC/ideoxyU/GCACGATTCATGCTCGA |
| | *T |
| SEQ ID No: 276 | ATCTCATCCCTGCGTGTC/ideoxyU/CCGAC/ideoxyU/GCACACATCCTGCATGT |
| | GA*T |
| SEQ ID No: 277 | ATCTCATCCCTGCGTGTC/ideoxyU/CCGAC/ideoxyU/GCACGCGCATCCTGCAT |
| | GA*T |
| SEQ ID No: 278 | ATCTCATCCCTGCGTGTC/ideoxyU/CCGAC/ideoxyU/GCACACTGCACGAATGA |
| | *T |
| SEQ ID No: 279 | ATCTCATCCCTGCGTGTC/ideoxyU/CCGAC/ideoxyU/GCACACGCACTGCCAGA |
| | *T |
| SEQ ID No: 280 | ATCTCATCCCTGCGTGTC/ideoxyU/CCGAC/ideoxyU/GCACTGCCATAGCACGA |
| | *T |
| SEQ ID No: 281 | ATCTCATCCCTGCGTGTC/ideoxyU/CCGAC/ideoxyU/GCACAGTTGTGCTGTGA |
| | *T |
| SEQ ID No: 282 | ATCTCATCCCTGCGTGTC/ideoxyU/CCGAC/ideoxyU/GCACATGGCGCGATCAT |
| | GA*T |
| SEQ ID No: 283 | ATCTCATCCCTGCGTGTC/ideoxyU/CCGAC/ideoxyU/GCACTTAGATATCATGA |
| | *T |
| SEQ ID No: 284 | ATCTCATCCCTGCGTGTC/ideoxyU/CCGAC/ideoxyU/GCACATCCTGTGCGCAT |
| | GA*T |
| SEQ ID No: 285 | ATCTCATCCCTGCGTGTC/ideoxyU/CCGAC/ideoxyU/GCACACGTGGCACATGA |
| | *T |
| SEQ ID No: 286 | ATCTCATCCCTGCGTGTC/ideoxyU/CCGAC/ideoxyU/GCACATGCGTCCTGTGA |
| | *T |
| SEQ ID No: 287 | ATCTCATCCCTGCGTGTC/ideoxyU/CCGAC/ideoxyU/GCACAATGCTCTGCATA |
| | GA*T |
| SEQ ID No: 288 | ATCTCATCCCTGCGTGTC/ideoxyU/CCGAC/ideoxyU/GCACTACATTGCACAGA |
| | *T |
| SEQ ID No: 289 | ATCTCATCCCTGCGTGTC/ideoxyU/CCGAC/ideoxyU/GCACATAGCGAGCCAGA |
| | *T |
| SEQ ID No: 290 | ATCTCATCCCTGCGTGTC/ideoxyU/CCGAC/ideoxyU/GCACGGAGTGCATGCAT |
| | GA*T |
| SEQ ID No: 291 | ATCTCATCCCTGCGTGTC/ideoxyU/CCGAC/ideoxyU/GCACATGGAGATACTGA |
| | *T |
| SEQ ID No: 292 | ATCTCATCCCTGCGTGTC/ideoxyU/CCGAC/ideoxyU/GCACTGAGCCTGTCAGA |
| | *T |
| SEQ ID No: 293 | ATCTCATCCCTGCGTGTC/ideoxyU/CCGAC/ideoxyU/GCACTCGAGATTGATGA |
| | *T |
| SEQ ID No: 294 | ATCTCATCCCTGCGTGTC/ideoxyU/CCGAC/ideoxyU/GCACATCCATCTCATCT |
| | GA*T |
| SEQ ID No: 295 | ATCTCATCCCTGCGTGTC/ideoxyU/CCGAC/ideoxyU/GCACACTGTCAGCCAGA |
| | *T |
| SEQ ID No: 296 | ATCTCATCCCTGCGTGTC/ideoxyU/CCGAC/ideoxyU/GCACAGACTTGCTGCGA |
| | *T |
| SEQ ID No: 297 | ATCTCATCCCTGCGTGTC/ideoxyU/CCGAC/ideoxyU/GCACATATAGCAGGAGA |
| | *T |
| SEQ ID No: 298 | ATCTCATCCCTGCGTGTC/ideoxyU/CCGAC/ideoxyU/GCACATGTCTAAGATGA |
| | *T |
| SEQ ID No: 299 | ATCTCATCCCTGCGTGTC/ideoxyU/CCGAC/ideoxyU/GCACATCGCAGCTGAAT |
| | GA*T |
| SEQ ID No: 300 | ATCTCATCCCTGCGTGTC/ideoxyU/CCGAC/ideoxyU/GCACTCTGTATTGCAGA |
| | *T |
| SEQ ID No: 301 | ATCTCATCCCTGCGTGTC/ideoxyU/CCGAC/ideoxyU/GCACATCATCGATTCGA |
| | *T |
| SEQ ID No: 302 | ATCTCATCCCTGCGTGTC/ideoxyU/CCGAC/ideoxyU/GCACTGTGAATATGCGA |
| | *T |
| SEQ ID No: 303 | ATCTCATCCCTGCGTGTC/ideoxyU/CCGAC/ideoxyU/GCACTGAATGATCTCGA |
| | *T |
| SEQ ID No: 304 | ATCTCATCCCTGCGTGTC/ideoxyU/CCGAC/ideoxyU/GCACAGAAGCTGCATGA |
| | GA*T |
| SEQ ID No: 305 | ATCTCATCCCTGCGTGTC/ideoxyU/CCGAC/ideoxyU/GCACTAGATTGCGCAGA |
| | *T |
| SEQ ID No: 306 | ATCTCATCCCTGCGTGTC/ideoxyU/CCGAC/ideoxyU/GCACGCATCCTCACAGA |
| | *T |
| SEQ ID No: 307 | ATCTCATCCCTGCGTGTC/ideoxyU/CCGAC/ideoxyU/GCACACAACATATCAGA |
| | *T |
| SEQ ID No: 308 | ATCTCATCCCTGCGTGTC/ideoxyU/CCGAC/ideoxyU/GCACATCCTGCATCGCA |
| | GA*T |
| SEQ ID No: 309 | ATCTCATCCCTGCGTGTC/ideoxyU/CCGAC/ideoxyU/GCACACATCAGCTCAAT |
| | GA*T |
| SEQ ID No: 310 | ATCTCATCCCTGCGTGTC/ideoxyU/CCGAC/ideoxyU/GCACATGCATATAATGC |
| | GA*T |
| SEQ ID No: 311 | ATCTCATCCCTGCGTGTC/ideoxyU/CCGAC/ideoxyU/GCACTCAATGCATCAGC |
| | GA*T |
| SEQ ID No: 312 | ATCTCATCCCTGCGTGTC/ideoxyU/CCGAC/ideoxyU/GCACAATGTGTGCTAGA |
| | *T |
| SEQ ID No: 313 | ATCTCATCCCTGCGTGTC/ideoxyU/CCGAC/ideoxyU/GCACTCAGCCTCATGCT |
| | GA*T |
| SEQ ID No: 314 | ATCTCATCCCTGCGTGTC/ideoxyU/CCGAC/ideoxyU/GCACTCTCGCATGCAAT |
| | GA*T |
| SEQ ID No: 315 | ATCTCATCCCTGCGTGTC/ideoxyU/CCGAC/ideoxyU/GCACTAGCAGCCAGCGA |
| | *T |
| SEQ ID No: 316 | ATCTCATCCCTGCGTGTC/ideoxyU/CCGAC/ideoxyU/GCACTGCCAGACTGTGA |
| | *T |
| SEQ ID No: 317 | ATCTCATCCCTGCGTGTC/ideoxyU/CCGAC/ideoxyU/GCACGCACTGCTCCTGA |
| | *T |
| SEQ ID No: 318 | ATCTCATCCCTGCGTGTC/ideoxyU/CCGAC/ideoxyU/GCACACATGGCAGCACA |
| | GA*T |
| SEQ ID No: 319 | ATCTCATCCCTGCGTGTC/ideoxyU/CCGAC/ideoxyU/GCACACTTGCAGATCGA |
| | *T |
| SEQ ID No: 320 | ATCTCATCCCTGCGTGTC/ideoxyU/CCGAC/ideoxyU/GCACTATGCCACAGCAT |
| | GA*T |
| SEQ ID No: 321 | ATCTCATCCCTGCGTGTC/ideoxyU/CCGAC/ideoxyU/GCACAACATCAGCATGA |
| | GA*T |
| SEQ ID No: 322 | ATCTCATCCCTGCGTGTC/ideoxyU/CCGAC/ideoxyU/GCACTGCAGTGATTCAT |
| | GA*T |
| SEQ ID No: 323 | ATCTCATCCCTGCGTGTC/ideoxyU/CCGAC/ideoxyU/GCACAGCACCTGCATCA |
| | GA*T |
| SEQ ID No: 324 | ATCTCATCCCTGCGTGTC/ideoxyU/CCGAC/ideoxyU/GCACTTATGCTATCAGA |
| | *T |
| SEQ ID No: 325 | ATCTCATCCCTGCGTGTC/ideoxyU/CCGAC/ideoxyU/GCACAATGCGTGCTGCA |
| | GA*T |
| SEQ ID No: 326 | ATCTCATCCCTGCGTGTC/ideoxyU/CCGAC/ideoxyU/GCACATCTCAGTGCAAT |
| | GA*T |
| SEQ ID No: 327 | ATCTCATCCCTGCGTGTC/ideoxyU/CCGAC/ideoxyU/GCACATGTGCTTCGCAT |
| | GA*T |
| SEQ ID No: 328 | ATCTCATCCCTGCGTGTC/ideoxyU/CCGAC/ideoxyU/GCACATGAGTGCAGCCA |
| | GA*T |
| SEQ ID No: 329 | ATCTCATCCCTGCGTGTC/ideoxyU/CCGAC/ideoxyU/GCACTCAAGCATGTGCT |
| | GA*T |
| SEQ ID No: 330 | ATCTCATCCCTGCGTGTC/ideoxyU/CCGAC/ideoxyU/GCACACAGTCAATGTGA |
| | *T |
| SEQ ID No: 331 | ATCTCATCCCTGCGTGTC/ideoxyU/CCGAC/ideoxyU/GCACACACTTGATGCGA |
| | *T |
| SEQ ID No: 332 | ATCTCATCCCTGCGTGTC/ideoxyU/CCGAC/ideoxyU/GCACATAGAGCCTCAGA |
| | *T |
| SEQ ID No: 333 | ATCTCATCCCTGCGTGTC/ideoxyU/CCGAC/ideoxyU/GCACTTGTGTCATGAGA |
| | *T |
| SEQ ID No: 334 | ATCTCATCCCTGCGTGTC/ideoxyU/CCGAC/ideoxyU/GCACAATGCACTCGAGA |
| | *T |
| SEQ ID No: 335 | ATCTCATCCCTGCGTGTC/ideoxyU/CCGAC/ideoxyU/GCACTTATGAGCGCTGA |
| | *T |
| SEQ ID No: 336 | ATCTCATCCCTGCGTGTC/ideoxyU/CCGAC/ideoxyU/GCACGTCATTGCACAGA |
| | *T |
| SEQ ID No: 337 | ATCTCATCCCTGCGTGTC/ideoxyU/CCGAC/ideoxyU/GCACATCACTGCAACGA |
| | *T |
| SEQ ID No: 338 | ATCTCATCCCTGCGTGTC/ideoxyU/CCGAC/ideoxyU/GCACTTGCATGCGATGC |
| | GA*T |
| SEQ ID No: 339 | ATCTCATCCCTGCGTGTC/ideoxyU/CCGAC/ideoxyU/GCACGTGCGCAAGCAGA |
| | *T |
| SEQ ID No: 340 | ATCTCATCCCTGCGTGTC/ideoxyU/CCGAC/ideoxyU/GCACTATCTCATAATGA |
| | *T |
| SEQ ID No: 341 | ATCTCATCCCTGCGTGTC/ideoxyU/CCGAC/ideoxyU/GCACATGGCTATGCACT |
| | GA*T |
| SEQ ID No: 342 | ATCTCATCCCTGCGTGTC/ideoxyU/CCGAC/ideoxyU/GCACTATGAATGAGCGA |
| | *T |
| SEQ ID No: 343 | ATCTCATCCCTGCGTGTC/ideoxyU/CCGAC/ideoxyU/GCACTATGCACCATCGA |
| | *T |
| SEQ ID No: 344 | ATCTCATCCCTGCGTGTC/ideoxyU/CCGAC/ideoxyU/GCACACAATGTGCGCGA |
| | *T |
| SEQ ID No: 345 | ATCTCATCCCTGCGTGTC/ideoxyU/CCGAC/ideoxyU/GCACAATGACTATCTGA |
| | *T |
| SEQ ID No: 346 | ATCTCATCCCTGCGTGTC/ideoxyU/CCGAC/ideoxyU/GCACAGCCGCTGCTCGA |
| | *T |
| SEQ ID No: 347 | ATCTCATCCCTGCGTGTC/ideoxyU/CCGAC/ideoxyU/GCACGCGCGCAGAATGA |
| | *T |
| SEQ ID No: 348 | ATCTCATCCCTGCGTGTC/ideoxyU/CCGAC/ideoxyU/GCACAGCCTCAGCGTGA |
| | *T |
| SEQ ID No: 349 | ATCTCATCCCTGCGTGTC/ideoxyU/CCGAC/ideoxyU/GCACTTGATCATACAGA |
| | *T |
| SEQ ID No: 350 | ATCTCATCCCTGCGTGTC/ideoxyU/CCGAC/ideoxyU/GCACTCTGCTGTGCAAT |
| | GA*T |
| SEQ ID No: 351 | ATCTCATCCCTGCGTGTC/ideoxyU/CCGAC/ideoxyU/GCACAGTGTATTGCTGA |
| | *T |
| SEQ ID No: 352 | ATCTCATCCCTGCGTGTC/ideoxyU/CCGAC/ideoxyU/GCACTGTGCATCTGCCT |
| | GA*T |
| SEQ ID No: 353 | ATCTCATCCCTGCGTGTC/ideoxyU/CCGAC/ideoxyU/GCACTCTATGTTGCTGA |
| | *T |
| SEQ ID No: 354 | ATCTCATCCCTGCGTGTC/ideoxyU/CCGAC/ideoxyU/GCACAACTATCTGCAGA |
| | *T |
| SEQ ID No: 355 | ATCTCATCCCTGCGTGTC/ideoxyU/CCGAC/ideoxyU/GCACAGATCTCATGAAT |
| | GA*T |
| SEQ ID No: 356 | ATCTCATCCCTGCGTGTC/ideoxyU/CCGAC/ideoxyU/GCACTATCATCCAGTGA |
| | *T |
| SEQ ID No: 357 | ATCTCATCCCTGCGTGTC/ideoxyU/CCGAC/ideoxyU/GCACTGCTACAAGCAGA |
| | *T |
| SEQ ID No: 358 | ATCTCATCCCTGCGTGTC/ideoxyU/CCGAC/ideoxyU/GCACAATATGCACGTGA |
| | *T |
| SEQ ID No: 359 | ATCTCATCCCTGCGTGTC/ideoxyU/CCGAC/ideoxyU/GCACACTTCTGCGATGA |
| | *T |
| SEQ ID No: 360 | ATCTCATCCCTGCGTGTC/ideoxyU/CCGAC/ideoxyU/GCACAAGCATATCTAGA |
| | *T |
| SEQ ID No: 361 | ATCTCATCCCTGCGTGTC/ideoxyU/CCGAC/ideoxyU/GCACAATGACAGCTCGA |
| | *T |
| SEQ ID No: 362 | ATCTCATCCCTGCGTGTC/ideoxyU/CCGAC/ideoxyU/GCACTGATGAGCTTGAT |
| | GA*T |
| SEQ ID No: 363 | ATCTCATCCCTGCGTGTC/ideoxyU/CCGAC/ideoxyU/GCACATATGACCTGAGA |
| | *T |
| SEQ ID No: 364 | ATCTCATCCCTGCGTGTC/ideoxyU/CCGAC/ideoxyU/GCACAGCCGATATCAGA |
| | *T |
| SEQ ID No: 365 | ATCTCATCCCTGCGTGTC/ideoxyU/CCGAC/ideoxyU/GCACAGTCAGTTGCAGA |
| | *T |
| SEQ ID No: 366 | ATCTCATCCCTGCGTGTC/ideoxyU/CCGAC/ideoxyU/GCACAACAGATGTATGA |
| | *T |
| SEQ ID No: 367 | ATCTCATCCCTGCGTGTC/ideoxyU/CCGAC/ideoxyU/GCACTCATCTGCGCAAT |
| | GA*T |
| SEQ ID No: 368 | ATCTCATCCCTGCGTGTC/ideoxyU/CCGAC/ideoxyU/GCACGGATCATGCGTGA |
| | *T |
| SEQ ID No: 369 | ATCTCATCCCTGCGTGTC/ideoxyU/CCGAC/ideoxyU/GCACTATTGCATGCTCT |
| | GA*T |
| SEQ ID No: 370 | ATCTCATCCCTGCGTGTC/ideoxyU/CCGAC/ideoxyU/GCACTACCTGATGCAGA |
| | *T |
| SEQ ID No: 371 | ATCTCATCCCTGCGTGTC/ideoxyU/CCGAC/ideoxyU/GCACATATAATCACAGA |
| | *T |
| SEQ ID No: 372 | ATCTCATCCCTGCGTGTC/ideoxyU/CCGAC/ideoxyU/GCACATGCAGCGCTAAT |
| | GA*T |
| SEQ ID No: 373 | ATCTCATCCCTGCGTGTC/ideoxyU/CCGAC/ideoxyU/GCACATGGCGCAGTGCT |
| | GA*T |
| SEQ ID No: 374 | ATCTCATCCCTGCGTGTC/ideoxyU/CCGAC/ideoxyU/GCACTGAGAATGTGTGA |
| | *T |
| SEQ ID No: 375 | ATCTCATCCCTGCGTGTC/ideoxyU/CCGAC/ideoxyU/GCACATGCATGGTACGA |
| | *T |
| SEQ ID No: 376 | ATCTCATCCCTGCGTGTC/ideoxyU/CCGAC/ideoxyU/GCACATGCATGCGAGGA |
| | GA*T |
| SEQ ID No: 377 | ATCTCATCCCTGCGTGTC/ideoxyU/CCGAC/ideoxyU/GCACAATCTGCATACGA |
| | *T |
| SEQ ID No: 378 | ATCTCATCCCTGCGTGTC/ideoxyU/CCGAC/ideoxyU/GCACAGCATGAGCGCCA |
| | GA*T |
| SEQ ID No: 379 | ATCTCATCCCTGCGTGTC/ideoxyU/CCGAC/ideoxyU/GCACTTATCTGATCTGA |
| | *T |
| SEQ ID No: 380 | ATCTCATCCCTGCGTGTC/ideoxyU/CCGAC/ideoxyU/GCACATCCAGCGCATGT |
| | GA*T |
| SEQ ID No: 381 | ATCTCATCCCTGCGTGTC/ideoxyU/CCGAC/ideoxyU/GCACAGTTCATCTGTGA |
| | *T |
| SEQ ID No: 382 | ATCTCATCCCTGCGTGTC/ideoxyU/CCGAC/ideoxyU/GCACAACATACATCAGA |
| | *T |
| SEQ ID No: 383 | ATCTCATCCCTGCGTGTC/ideoxyU/CCGAC/ideoxyU/GCACGGCTAGATGCAGA |
| | *T |
| SEQ ID No: 384 | ATCTCATCCCTGCGTGTC/ideoxyU/CCGAC/ideoxyU/GCACAGTTATGTGCTGA |
| | *T |
| SEQ ID No: 385 | ATCTCATCCCTGCGTGTC/ideoxyU/CCGAC/ideoxyU/GCACTGCCGAGCAGCAT |
| | GA*T |
| SEQ ID No: 386 | ATCTCATCCCTGCGTGTC/ideoxyU/CCGAC/ideoxyU/GCACTGCCTCAGATCAT |
| | GA*T |
| SEQ ID No: 387 | ATCTCATCCCTGCGTGTC/ideoxyU/CCGAC/ideoxyU/GCACTGATCAGTGGCGA |
| | *T |
| SEQ ID No: 388 | ATCTCATCCCTGCGTGTC/ideoxyU/CCGAC/ideoxyU/GCACTGCTGCGGAGCAT |
| | GA*T |
| SEQ ID No: 389 | ATCTCATCCCTGCGTGTC/ideoxyU/CCGAC/ideoxyU/GCACAGTGCTGTGGCAT |
| | GA*T |
| SEQ ID No: 390 | ATCTCATCCCTGCGTGTC/ideoxyU/CCGAC/ideoxyU/GCACACATATGGCATAT |
| | GA*T |
| SEQ ID No: 391 | ATCTCATCCCTGCGTGTC/ideoxyU/CCGAC/ideoxyU/GCACAGATCGCCACAGA |
| | *T |
| SEQ ID No: 392 | ATCTCATCCCTGCGTGTC/ideoxyU/CCGAC/ideoxyU/GCACACGGCTCAGATGA |
| | *T |
| SEQ ID No: 393 | ATCTCATCCCTGCGTGTC/ideoxyU/CCGAC/ideoxyU/GCACTTGATGATAGTGA |
| | *T |
| SEQ ID No: 394 | ATCTCATCCCTGCGTGTC/ideoxyU/CCGAC/ideoxyU/GCACAACTGCACATAGA |
| | *T |
| SEQ ID No: 395 | ATCTCATCCCTGCGTGTC/ideoxyU/CCGAC/ideoxyU/GCACGCATACATCCTGA |
| | *T |
| SEQ ID No: 396 | ATCTCATCCCTGCGTGTC/ideoxyU/CCGAC/ideoxyU/GCACATCTCTGGCTGCA |
| | GA*T |
| SEQ ID No: 397 | ATCTCATCCCTGCGTGTC/ideoxyU/CCGAC/ideoxyU/GCACATCTGGTGCATGT |
| | GA*T |
| SEQ ID No: 398 | ATCTCATCCCTGCGTGTC/ideoxyU/CCGAC/ideoxyU/GCACAGCAGCTTCGCGA |
| | *T |
| SEQ ID No: 399 | ATCTCATCCCTGCGTGTC/ideoxyU/CCGAC/ideoxyU/GCACGCATATGGCAGCA |
| | GA*T |
| SEQ ID No: 400 | ATCTCATCCCTGCGTGTC/ideoxyU/CCGAC/ideoxyU/GCACACAGCACCGCTGA |
| | *T |
| SEQ ID No: 401 | ATCTCATCCCTGCGTGTC/ideoxyU/CCGAC/ideoxyU/GCACTAGCATCTGGTGA |
| | *T |
| SEQ ID No: 402 | ATCTCATCCCTGCGTGTC/ideoxyU/CCGAC/ideoxyU/GCACTTCAGCATACAGA |
| | *T |
| SEQ ID No: 403 | ATCTCATCCCTGCGTGTC/ideoxyU/CCGAC/ideoxyU/GCACATGCAGATGGCGA |
| | GA*T |
| SEQ ID No: 404 | ATCTCATCCCTGCGTGTC/ideoxyU/CCGAC/ideoxyU/GCACAGATCTATCCAGA |
| | *T |
| SEQ ID No: 405 | ATCTCATCCCTGCGTGTC/ideoxyU/CCGAC/ideoxyU/GCACTTCATGCATCTCA |
| | GA*T |
| SEQ ID No: 406 | ATCTCATCCCTGCGTGTC/ideoxyU/CCGAC/ideoxyU/GCACATGCAAGTGTGAT |
| | GA*T |
| SEQ ID No: 407 | ATCTCATCCCTGCGTGTC/ideoxyU/CCGAC/ideoxyU/GCACTGTTCGCTGCAGA |
| | *T |
| SEQ ID No: 408 | ATCTCATCCCTGCGTGTC/ideoxyU/CCGAC/ideoxyU/GCACTCAGATCCTGCAT |
| | GA*T |
| SEQ ID No: 409 | ATCTCATCCCTGCGTGTC/ideoxyU/CCGAC/ideoxyU/GCACACACAGATAATGA |
| | *T |
| SEQ ID No: 410 | ATCTCATCCCTGCGTGTC/ideoxyU/CCGAC/ideoxyU/GCACGATGCTCTGGCGA |
| | *T |
| SEQ ID No: 411 | ATCTCATCCCTGCGTGTC/ideoxyU/CCGAC/ideoxyU/GCACAGATCCATCATCT |
| | GA*T |
| SEQ ID No: 412 | ATCTCATCCCTGCGTGTC/ideoxyU/CCGAC/ideoxyU/GCACTGCACCATCATAT |
| | GA*T |
| SEQ ID No: 413 | ATCTCATCCCTGCGTGTC/ideoxyU/CCGAC/ideoxyU/GCACATGCTATAGCAAT |
| | GA*T |
| SEQ ID No: 414 | ATCTCATCCCTGCGTGTC/ideoxyU/CCGAC/ideoxyU/GCACATCTGCACATGGC |
| | GA*T |
| SEQ ID No: 415 | ATCTCATCCCTGCGTGTC/ideoxyU/CCGAC/ideoxyU/GCACGCTGCATGAAGAT |
| | GA*T |
| SEQ ID No: 416 | ATCTCATCCCTGCGTGTC/ideoxyU/CCGAC/ideoxyU/GCACTATTCAGATGCAT |
| | GA*T |
| SEQ ID No: 417 | ATCTCATCCCTGCGTGTC/ideoxyU/CCGAC/ideoxyU/GCACTCGCGCAATGCGA |
| | *T |
| SEQ ID No: 418 | ATCTCATCCCTGCGTGTC/ideoxyU/CCGAC/ideoxyU/GCACGCGCAGATGGCAT |
| | GA*T |
| SEQ ID No: 419 | ATCTCATCCCTGCGTGTC/ideoxyU/CCGAC/ideoxyU/GCACAGTGAATCATCGA |
| | *T |
| SEQ ID No: 420 | ATCTCATCCCTGCGTGTC/ideoxyU/CCGAC/ideoxyU/GCACATGCAACACTAGA |
| | *T |
| SEQ ID No: 421 | ATCTCATCCCTGCGTGTC/ideoxyU/CCGAC/ideoxyU/GCACTATTATGATATGA |
| | *T |
| SEQ ID No: 422 | ATCTCATCCCTGCGTGTC/ideoxyU/CCGAC/ideoxyU/GCACTCAGAAGCATCGA |
| | *T |
| SEQ ID No: 423 | ATCTCATCCCTGCGTGTC/ideoxyU/CCGAC/ideoxyU/GCACTTCATATCTGAGA |
| | *T |
| SEQ ID No: 424 | ATCTCATCCCTGCGTGTC/ideoxyU/CCGAC/ideoxyU/GCACAATAGCTATGAGA |
| | *T |
| SEQ ID No: 425 | ATCTCATCCCTGCGTGTC/ideoxyU/CCGAC/ideoxyU/GCACATGGATAGCTGCA |
| | GA*T |
| SEQ ID No: 426 | ATCTCATCCCTGCGTGTC/ideoxy U/CCGAC/ideoxyU/GCACATAATATCTGCG |
| | A*T |
| SEQ ID No: 427 | ATCTCATCCCTGCGTGTC/ideoxyU/CCGAC/ideoxyU/GCACGCATGCATCCTGA |
| | GA*T |
| SEQ ID No: 428 | ATCTCATCCCTGCGTGTC/ideoxyU/CCGAC/ideoxyU/GCACAGCACAGAGGAGA |
| | *T |
| SEQ ID No: 429 | ATCTCATCCCTGCGTGTC/ideoxyU/CCGAC/ideoxyU/GCACAACTGCGCTCTGA |
| | *T |
| SEQ ID No: 430 | ATCTCATCCCTGCGTGTC/ideoxyU/CCGAC/ideoxyU/GCACACAAGATGACAGA |
| | *T |
| SEQ ID No: 431 | ATCTCATCCCTGCGTGTC/ideoxyU/CCGAC/ideoxyU/GCACAGATGACATTAGA |
| | *T |
| SEQ ID No: 432 | ATCTCATCCCTGCGTGTC/ideoxyU/CCGAC/ideoxyU/GCACATGGCTCACATAT |
| | GA*T |
| SEQ ID No: 433 | ATCTCATCCCTGCGTGTC/ideoxyU/CCGAC/ideoxyU/GCACAAGAGATGCATCT |
| | GA*T |
| SEQ ID No: 434 | ATCTCATCCCTGCGTGTC/ideoxyU/CCGAC/ideoxyU/GCACATGACCGCTGTGA |
| | *T |
| SEQ ID No: 435 | ATCTCATCCCTGCGTGTC/ideoxyU/CCGAC/ideoxyU/GCACGCATCTGGCGCGA |
| | *T |
| SEQ ID No: 436 | ATCTCATCCCTGCGTGTC/ideoxyU/CCGAC/ideoxyU/GCACTTAGCTGTGATGA |
| | *T |
| SEQ ID No: 437 | ATCTCATCCCTGCGTGTC/ideoxyU/CCGAC/ideoxyU/GCACTCGCAATAGATGA |
| | *T |
| SEQ ID No: 438 | ATCTCATCCCTGCGTGTC/ideoxyU/CCGAC/ideoxyU/GCACACTCTCTCAATGA |
| | *T |
| SEQ ID No: 439 | ATCTCATCCCTGCGTGTC/ideoxyU/CCGAC/ideoxyU/GCACACTGCCTGATGAT |
| | GA*T |
| SEQ ID No: 440 | ATCTCATCCCTGCGTGTC/ideoxyU/CCGAC/ideoxyU/GCACTCTATTCATATGA |
| | *T |
| SEQ ID No: 441 | ATCTCATCCCTGCGTGTC/ideoxyU/CCGAC/ideoxyU/GCACTTGAGCGCACTGA |
| | *T |
| SEQ ID No: 442 | ATCTCATCCCTGCGTGTC/ideoxyU/CCGAC/ideoxyU/GCACGCTGATCAGGCGA |
| | *T |
| SEQ ID No: 443 | ATCTCATCCCTGCGTGTC/ideoxyU/CCGAC/ideoxyU/GCACGCTCTGTTGATGA |
| | *T |
| SEQ ID No: 444 | ATCTCATCCCTGCGTGTC/ideoxyU/CCGAC/ideoxyU/GCACTCAACATATCTGA |
| | *T |
| SEQ ID No: 445 | ATCTCATCCCTGCGTGTC/ideoxyU/CCGAC/ideoxyU/GCACATGCGGCGCAGCA |
| | GA*T |
| SEQ ID No: 446 | ATCTCATCCCTGCGTGTC/ideoxyU/CCGAC/ideoxyU/GCACTTGCTGTGCGCGA |
| | *T |
| SEQ ID No: 447 | ATCTCATCCCTGCGTGTC/ideoxyU/CCGAC/ideoxyU/GCACATAGCGTTCATGA |
| | *T |
| SEQ ID No: 448 | ATCTCATCCCTGCGTGTC/ideoxyU/CCGAC/ideoxyU/GCACGCACATAGCCTGA |
| | *T |
| SEQ ID No: 449 | ATCTCATCCCTGCGTGTC/ideoxyU/CCGAC/ideoxyU/GCACTTCTCATGTCAGA |
| | *T |
| SEQ ID No: 450 | ATCTCATCCCTGCGTGTC/ideoxyU/CCGAC/ideoxyU/GCACAGCCATCTGCTGA |
| | GA*T |
| SEQ ID No: 451 | ATCTCATCCCTGCGTGTC/ideoxyU/CCGAC/ideoxyU/GCACACTCAGCGCCAGA |
| | *T |
| SEQ ID No: 452 | ATCTCATCCCTGCGTGTC/ideoxyU/CCGAC/ideoxyU/GCACAACATCTGATAGA |
| | *T |
| SEQ ID No: 453 | ATCTCATCCCTGCGTGTC/ideoxyU/CCGAC/ideoxyU/GCACTGCTCCGCATCGA |
| | *T |
| SEQ ID No: 454 | ATCTCATCCCTGCGTGTC/ideoxyU/CCGAC/ideoxyU/GCACTATCGCTTGATGA |
| | *T |
| SEQ ID No: 455 | ATCTCATCCCTGCGTGTC/ideoxyU/CCGAC/ideoxyU/GCACTCTTCTCAGCAGA |
| | *T |
| SEQ ID No: 456 | ATCTCATCCCTGCGTGTC/ideoxyU/CCGAC/ideoxyU/GCACATGGTGTGCACGA |
| | *T |
| SEQ ID No: 457 | ATCTCATCCCTGCGTGTC/ideoxyU/CCGAC/ideoxyU/GCACATATCATCTTAGA |
| | *T |
| SEQ ID No: 458 | ATCTCATCCCTGCGTGTC/ideoxyU/CCGAC/ideoxyU/GCACGGTGCTGCTATGA |
| | *T |
| SEQ ID No: 459 | ATCTCATCCCTGCGTGTC/ideoxyU/CCGAC/ideoxyU/GCACTGTCAGCCTATGA |
| | *T |
| SEQ ID No: 460 | ATCTCATCCCTGCGTGTC/ideoxyU/CCGAC/ideoxyU/GCACATGATCACACAAT |
| | GA*T |

TABLE 10
PCR-free adapter sequences

| SEQ ID No. | Sequence |
|---|---|
| SEQ ID No: 461 | TCGCATTCGAGCTGTGCAGTCGGAGACACGCAGGGA/ideoxyU/GAGA/ideoxyU/GG/ideoxyU |
| SEQ ID No: 462 | TCGATGGCTGCACATGTGCAGTCGGAGACACGCAGGGA/ideoxyU/GAGA/ideoxyU/GG/ideoxyU |
| SEQ ID No: 463 | TCATTCATGTGTGATGTGCAGTCGGAGACACGCAGGGA/ideoxyU/GAGA/ideoxyU/GG/ideoxyU |
| SEQ ID No: 464 | TCATGCCTACACAGTGCAGTCGGAGACACGCAGGGA/ideoxyU/GAGA/ideoxyU/GG/ideoxyU |
| SEQ ID No: 465 | TCAGAGGATACATGTGCAGTCGGAGACACGCAGGGA/ideoxyU/GAGA/ideoxyU/GG/ideoxyU |
| SEQ ID No: 466 | TCATAGGCTATATGTGCAGTCGGAGACACGCAGGGA/ideoxyU/GAGA/ideoxyU/GG/ideoxyU |
| SEQ ID No: 467 | TCGAGCATGAATCGTGCAGTCGGAGACACGCAGGGA/ideoxyU/GAGA/ideoxyU/GG/ideoxyU |
| SEQ ID No: 468 | TCACATGCAGGATGTGTGCAGTCGGAGACACGCAGGGA/ideoxyU/GAGA/ideoxyU/GG/ideoxyU |
| SEQ ID No: 469 | TCATGCAGGATGCGCGTGCAGTCGGAGACACGCAGGGA/ideoxyU/GAGA/ideoxyU/GG/ideoxyU |
| SEQ ID No: 470 | TCATTCGTGCAGTGTGCAGTCGGAGACACGCAGGGA/ideoxyU/GAGA/ideoxyU/GG/ideoxyU |
| SEQ ID No: 471 | TCTGGCAGTGCGTGTGCAGTCGGAGACACGCAGGGA/ideoxyU/GAGA/ideoxyU/GG/ideoxyU |
| SEQ ID No: 472 | TCGTGCTATGGCAGTGCAGTCGGAGACACGCAGGGA/ideoxyU/GAGA/ideoxyU/GG/ideoxyU |
| SEQ ID No: 473 | TCACAGCACAACTGTGCAGTCGGAGACACGCAGGGA/ideoxyU/GAGA/ideoxyU/GG/ideoxyU |
| SEQ ID No: 474 | TCATGATCGCGCCATGTGCAGTCGGAGACACGCAGGGA/ideoxyU/GAGA/ideoxyU/GG/ideoxyU |
| SEQ ID No: 475 | TCATGATATCTAAGTGCAGTCGGAGACACGCAGGGA/ideoxyU/GAGA/ideoxyU/GG/ideoxyU |
| SEQ ID No: 476 | TCATGCGCACAGGATGTGCAGTCGGAGACACGCAGGGA/ideoxyU/GAGA/ideoxyU/GG/ideoxyU |
| SEQ ID No: 477 | TCATGTGCCACGTGTGCAGTCGGAGACACGCAGGGA/ideoxyU/GAGA/ideoxyU/GG/ideoxyU |
| SEQ ID No: 478 | TCACAGGACGCATGTGCAGTCGGAGACACGCAGGGA/ideoxyU/GAGA/ideoxyU/GG/ideoxyU |
| SEQ ID No: 479 | TCTATGCAGAGCATTGTGCAGTCGGAGACACGCAGGGA/ideoxyU/GAGA/ideoxyU/GG/ideoxyU |
| SEQ ID No: 480 | TCTGTGCAATGTAGTGCAGTCGGAGACACGCAGGGA/ideoxyU/GAGA/ideoxyU/GG/ideoxyU |
| SEQ ID No: 481 | TCTGGCTCGCTATGTGCAGTCGGAGACACGCAGGGA/ideoxyU/GAGA/ideoxyU/GG/ideoxyU |
| SEQ ID No: 482 | TCATGCATGCACTCCGTGCAGTCGGAGACACGCAGGGA/ideoxyU/GAGA/ideoxyU/GG/ideoxyU |
| SEQ ID No: 483 | TCAGTATCTCCATGTGCAGTCGGAGACACGCAGGGA/ideoxyU/GAGA/ideoxyU/GG/ideoxyU |
| SEQ ID No: 484 | TCTGACAGGCTCAGTGCAGTCGGAGACACGCAGGGA/ideoxyU/GAGA/ideoxyU/GG/ideoxyU |
| SEQ ID No: 485 | TCATCAATCTCGAGTGCAGTCGGAGACACGCAGGGA/ideoxyU/GAGA/ideoxyU/GG/ideoxyU |
| SEQ ID No: 486 | TCAGATGAGATGGATGTGCAGTCGGAGACACGCAGGGA/ideoxyU/GAGA/ideoxyU/GG/ideoxyU |
| SEQ ID No: 487 | TCTGGCTGACAGTGTGCAGTCGGAGACACGCAGGGA/ideoxyU/GAGA/ideoxyU/GG/ideoxyU |
| SEQ ID No: 488 | TCGCAGCAAGTCTGTGCAGTCGGAGACACGCAGGGA/ideoxyU/GAGA/ideoxyU/GG/ideoxyU |
| SEQ ID No: 489 | TCTCCTGCTATATGTGCAGTCGGAGACACGCAGGGA/ideoxyU/GAGA/ideoxyU/GG/ideoxyU |
| SEQ ID No: 490 | TCATCTTAGACATGTGCAGTCGGAGACACGCAGGGA/ideoxyU/GAGA/ideoxyU/GG/ideoxyU |
| SEQ ID No: 491 | TCATTCAGCTGCGATGTGCAGTCGGAGACACGCAGGGA/ideoxyU/GAGA/ideoxyU/GG/ideoxyU |
| SEQ ID No: 492 | TCTGCAATACAGAGTGCAGTCGGAGACACGCAGGGA/ideoxyU/GAGA/ideoxyU/GG/ideoxyU |
| SEQ ID No: 493 | TCGAATCGATGATGTGCAGTCGGAGACACGCAGGGA/ideoxyU/GAGA/ideoxyU/GG/ideoxyU |
| SEQ ID No: 494 | TCGCATATTCACAGTGCAGTCGGAGACACGCAGGGA/ideoxyU/GAGA/ideoxyU/GG/ideoxyU |
| SEQ ID No: 495 | TCGAGATCATTCAGTGCAGTCGGAGACACGCAGGGA/ideoxyU/GAGA/ideoxyU/GG/ideoxyU |
| SEQ ID No: 496 | TCTCATGCAGCTTCTGTGCAGTCGGAGACACGCAGGGA/ideoxyU/GAGA/ideoxyU/GG/ideoxyU |
| SEQ ID No: 497 | TCTGCGCAATCTAGTGCAGTCGGAGACACGCAGGGA/ideoxyU/GAGA/ideoxyU/GG/ideoxyU |
| SEQ ID No: 498 | TCTGTGAGGATGCGTGCAGTCGGAGACACGCAGGGA/ideoxyU/GAGA/ideoxyU/GG/ideoxyU |
| SEQ ID No: 499 | TCTGATATGTTGTGTGCAGTCGGAGACACGCAGGGA/ideoxyU/GAGA/ideoxyU/GG/ideoxyU |
| SEQ ID No: 500 | TCTGCGATGCAGGATGTGCAGTCGGAGACACGCAGGGA/ideoxyU/GAGA/ideoxyU/GG/ideoxyU |
| SEQ ID No: 501 | TCATTGAGCTGATGTGTGCAGTCGGAGACACGCAGGGA/ideoxyU/GAGA/ideoxyU/GG/ideoxyU |
| SEQ ID No: 502 | TCGCATTATATGCATGTGCAGTCGGAGACACGCAGGGA/ideoxyU/GAGA/ideoxyU/GG/ideoxyU |
| SEQ ID No: 503 | TCGCTGATGCATTGAGTGCAGTCGGAGACACGCAGGGA/ideoxyU/GAGA/ideoxyU/GG/ideoxyU |
| SEQ ID No: 504 | TCTAGCACACATTGTGCAGTCGGAGACACGCAGGGA/ideoxyU/GAGA/ideoxyU/GG/ideoxyU |
| SEQ ID No: 505 | TCAGCATGAGGCTGAGTGCAGTCGGAGACACGCAGGGA/ideoxyU/GAGA/ideoxyU/GG/ideoxyU |
| SEQ ID No: 506 | TCATTGCATGCGAGAGTGCAGTCGGAGACACGCAGGGA/ideoxyU/GAGA/ideoxyU/GG/ideoxyU |
| SEQ ID No: 507 | TCGCTGGCTGCTAGTGCAGTCGGAGACACGCAGGGA/ideoxyU/GAGA/ideoxyU/GG/ideoxyU |
| SEQ ID No: 508 | TCACAGTCTGGCAGTGCAGTCGGAGACACGCAGGGA/ideoxyU/GAGA/ideoxyU/GG/ideoxyU |
| SEQ ID No: 509 | TCAGGAGCAGTGCGTGCAGTCGGAGACACGCAGGGA/ideoxyU/GAGA/ideoxyU/GG/ideoxyU |
| SEQ ID No: 510 | TCTGTGCTGCCATGTGTGCAGTCGGAGACACGCAGGGA/ideoxyU/GAGA/ideoxyU/GG/ideoxyU |
| SEQ ID No: 511 | TCGATCTGCAAGTGTGCAGTCGGAGACACGCAGGGA/ideoxyU/GAGA/ideoxyU/GG/ideoxyU |
| SEQ ID No: 512 | TCATGCTGTGGCATAGTGCAGTCGGAGACACGCAGGGA/ideoxyU/GAGA/ideoxyU/GG/ideoxyU |
| SEQ ID No: 513 | TCTCATGCTGATGTTGTGCAGTCGGAGACACGCAGGGA/ideoxyU/GAGA/ideoxyU/GG/ideoxyU |
| SEQ ID No: 514 | TCATGAATCACTGCAGTGCAGTCGGAGACACGCAGGGA/ideoxyU/GAGA/ideoxyU/GG/ideoxyU |
| SEQ ID No: 515 | TCTGATGCAGGTGCTGTGCAGTCGGAGACACGCAGGGA/ideoxyU/GAGA/ideoxyU/GG/ideoxyU |
| SEQ ID No: 516 | TCTGATAGCATAAGTGCAGTCGGAGACACGCAGGGA/ideoxyU/GAGA/ideoxyU/GG/ideoxyU |
| SEQ ID No: 517 | TCTGCAGCACGCATTGTGCAGTCGGAGACACGCAGGGA/ideoxyU/GAGA/ideoxyU/GG/ideoxyU |
| SEQ ID No: 518 | TCATTGCACTGAGATGTGCAGTCGGAGACACGCAGGGA/ideoxyU/GAGA/ideoxyU/GG/ideoxyU |
| SEQ ID No: 519 | TCATGCGAAGCACATGTGCAGTCGGAGACACGCAGGGA/ideoxyU/GAGA/ideoxyU/GG/ideoxyU |
| SEQ ID No: 520 | TCTGGCTGCACTCATGTGCAGTCGGAGACACGCAGGGA/ideoxyU/GAGA/ideoxyU/GG/ideoxyU |
| SEQ ID No: 521 | TCAGCACATGCTTGAGTGCAGTCGGAGACACGCAGGGA/ideoxyU/GAGA/ideoxyU/GG/ideoxyU |
| SEQ ID No: 522 | TCACATTGACTGTGTGCAGTCGGAGACACGCAGGGA/ideoxyU/GAGA/ideoxyU/GG/ideoxyU |
| SEQ ID No: 523 | TCGCATCAAGTGTGTGCAGTCGGAGACACGCAGGGA/ideoxyU/GAGA/ideoxyU/GG/ideoxyU |
| SEQ ID No: 524 | TCTGAGGCTCTATGTGCAGTCGGAGACACGCAGGGA/ideoxyU/GAGA/ideoxyU/GG/ideoxyU |
| SEQ ID No: 525 | TCTCATGACACAAGTGCAGTCGGAGACACGCAGGGA/ideoxyU/GAGA/ideoxyU/GG/ideoxyU |
| SEQ ID No: 526 | TCTCGAGTGCATTGTGCAGTCGGAGACACGCAGGGA/ideoxyU/GAGA/ideoxyU/GG/ideoxyU |
| SEQ ID No: 527 | TCAGCGCTCATAAGTGCAGTCGGAGACACGCAGGGA/ideoxyU/GAGA/ideoxyU/GG/ideoxyU |
| SEQ ID No: 528 | TCTGTGCAATGACGTGCAGTCGGAGACACGCAGGGA/ideoxyU/GAGA/ideoxyU/GG/ideoxyU |
| SEQ ID No: 529 | TCGTTGCAGTGATGTGCAGTCGGAGACACGCAGGGA/ideoxyU/GAGA/ideoxyU/GG/ideoxyU |
| SEQ ID No: 530 | TCGCATCGCATGCAAGTGCAGTCGGAGACACGCAGGGA/ideoxyU/GAGA/ideoxyU/GG/ideoxyU |
| SEQ ID No: 531 | TCTGCTTGCGCACGTGCAGTCGGAGACACGCAGGGA/ideoxyU/GAGA/ideoxyU/GG/ideoxyU |
| SEQ ID No: 532 | TCATTATGAGATAGTGCAGTCGGAGACACGCAGGGA/ideoxyU/GAGA/ideoxyU/GG/ideoxyU |
| SEQ ID No: 533 | TCAGTGCATAGCCATGTGCAGTCGGAGACACGCAGGGA/ideoxyU/GAGA/ideoxyU/GG/ideoxyU |
| SEQ ID No: 534 | TCGCTCATTCATAGTGCAGTCGGAGACACGCAGGGA/ideoxyU/GAGA/ideoxyU/GG/ideoxyU |
| SEQ ID No: 535 | TCGATGGTGCATAGTGCAGTCGGAGACACGCAGGGA/ideoxyU/GAGA/ideoxyU/GG/ideoxyU |
| SEQ ID No: 536 | TCGCGCACATTGTGTGCAGTCGGAGACACGCAGGGA/ideoxyU/GAGA/ideoxyU/GG/ideoxyU |
| SEQ ID No: 537 | TCAGATAGTCATTGTGCAGTCGGAGACACGCAGGGA/ideoxyU/GAGA/ideoxyU/GG/ideoxyU |
| SEQ ID No: 538 | TCGAGCAGCGGCTGTGCAGTCGGAGACACGCAGGGA/ideoxyU/GAGA/ideoxyU/GG/ideoxyU |
| SEQ ID No: 539 | TCATTCTGCGCGCGTGCAGTCGGAGACACGCAGGGA/ideoxyU/GAGA/ideoxyU/GG/ideoxyU |
| SEQ ID No: 540 | TCACGCTGAGGCTGTGCAGTCGGAGACACGCAGGGA/ideoxyU/GAGA/ideoxyU/GG/ideoxyU |
| SEQ ID No: 541 | TCTGTATGATCAAGTGCAGTCGGAGACACGCAGGGA/ideoxyU/GAGA/ideoxyU/GG/ideoxyU |
| SEQ ID No: 542 | TCATTGCACAGCAGAGTGCAGTCGGAGACACGCAGGGA/ideoxyU/GAGA/ideoxyU/GG/ideoxyU |
| SEQ ID No: 543 | TCAGCAATACACTGTGCAGTCGGAGACACGCAGGGA/ideoxyU/GAGA/ideoxyU/GG/ideoxyU |
| SEQ ID No: 544 | TCAGGCAGATGCACAGTGCAGTCGGAGACACGCAGGGA/ideoxyU/GAGA/ideoxyU/GG/ideoxyU |
| SEQ ID No: 545 | TCAGCAACATAGAGTGCAGTCGGAGACACGCAGGGA/ideoxyU/GAGA/ideoxyU/GG/ideoxyU |
| SEQ ID No: 546 | TCTGCAGATAGTTGTGCAGTCGGAGACACGCAGGGA/ideoxyU/GAGA/ideoxyU/GG/ideoxyU |
| SEQ ID No: 547 | TCATTCATGAGATCTGTGCAGTCGGAGACACGCAGGGA/ideoxyU/GAGA/ideoxyU/GG/ideoxyU |
| SEQ ID No: 548 | TCACTGGATGATAGTGCAGTCGGAGACACGCAGGGA/ideoxyU/GAGA/ideoxyU/GG/ideoxyU |
| SEQ ID No: 549 | TCTGCTTGTAGCAGTGCAGTCGGAGACACGCAGGGA/ideoxyU/GAGA/ideoxyU/GG/ideoxyU |
| SEQ ID No: 550 | TCACGTGCATATTGTGCAGTCGGAGACACGCAGGGA/ideoxyU/GAGA/ideoxyU/GG/ideoxyU |
| SEQ ID No: 551 | TCATCGCAGAAGTGTGCAGTCGGAGACACGCAGGGA/ideoxyU/GAGA/ideoxyU/GG/ideoxyU |
| SEQ ID No: 552 | TCTAGATATGCTTGTGCAGTCGGAGACACGCAGGGA/ideoxyU/GAGA/ideoxyU/GG/ideoxyU |
| SEQ ID No: 553 | TCGAGCTGTCATTGTGCAGTCGGAGACACGCAGGGA/ideoxyU/GAGA/ideoxyU/GG/ideoxyU |
| SEQ ID No: 554 | TCATCAAGCTCATCAGTGCAGTCGGAGACACGCAGGGA/ideoxyU/GAGA/ideoxyU/GG/ideoxyU |
| SEQ ID No: 555 | TCTCAGGTCATATGTGCAGTCGGAGACACGCAGGGA/ideoxyU/GAGA/ideoxyU/GG/ideoxyU |
| SEQ ID No: 556 | TCTGATATCGGCTGTGCAGTCGGAGACACGCAGGGA/ideoxyU/GAGA/ideoxyU/GG/ideoxyU |
| SEQ ID No: 557 | TCTGCAACTGACTGTGCAGTCGGAGACACGCAGGGA/ideoxyU/GAGA/ideoxyU/GG/ideoxyU |
| SEQ ID No: 558 | TCATACATCTGTTGTGCAGTCGGAGACACGCAGGGA/ideoxyU/GAGA/ideoxyU/GG/ideoxyU |
| SEQ ID No: 559 | TCATTGCGCAGATGAGTGCAGTCGGAGACACGCAGGGA/ideoxyU/GAGA/ideoxyU/GG/ideoxyU |
| SEQ ID No: 560 | TCACGCATGATCCGTGCAGTCGGAGACACGCAGGGA/ideoxyU/GAGA/ideoxyU/GG/ideoxyU |
| SEQ ID No: 561 | TCAGAGCATGCAATAGTGCAGTCGGAGACACGCAGGGA/ideoxyU/GAGA/ideoxyU/GG/ideoxyU |
| SEQ ID No: 562 | TCTGCATCAGGTAGTGCAGTCGGAGACACGCAGGGA/ideoxyU/GAGA/ideoxyU/GG/ideoxyU |
| SEQ ID No: 563 | TCTGTGATTATATGTGCAGTCGGAGACACGCAGGGA/ideoxyU/GAGA/ideoxyU/GG/ideoxyU |
| SEQ ID No: 564 | TCATTAGCGCTGCATGTGCAGTCGGAGACACGCAGGGA/ideoxyU/GAGA/ideoxyU/GG/ideoxyU |
| SEQ ID No: 565 | TCAGCACTGCGCCATGTGCAGTCGGAGACACGCAGGGA/ideoxyU/GAGA/ideoxyU/GG/ideoxyU |
| SEQ ID No: 566 | TCACACATTCTCAGTGCAGTCGGAGACACGCAGGGA/ideoxyU/GAGA/ideoxyU/GG/ideoxyU |
| SEQ ID No: 567 | TCGTACCATGCATGTGCAGTCGGAGACACGCAGGGA/ideoxyU/GAGA/ideoxyU/GG/ideoxyU |
| SEQ ID No: 568 | TCTCCTCGCATGCATGTGCAGTCGGAGACACGCAGGGA/ideoxyU/GAGA/ideoxyU/GG/ideoxyU |
| SEQ ID No: 569 | TCGTATGCAGATTGTGCAGTCGGAGACACGCAGGGA/ideoxyU/GAGA/ideoxyU/GG/ideoxyU |
| SEQ ID No: 570 | TCTGGCGCTCATGCTGTGCAGTCGGAGACACGCAGGGA/ideoxyU/GAGA/ideoxyU/GG/ideoxyU |
| SEQ ID No: 571 | TCAGATCAGATAAGTGCAGTCGGAGACACGCAGGGA/ideoxyU/GAGA/ideoxyU/GG/ideoxyU |
| SEQ ID No: 572 | TCACATGCGCTGGATGTGCAGTCGGAGACACGCAGGGA/ideoxyU/GAGA/ideoxyU/GG/ideoxyU |
| SEQ ID No: 573 | TCACAGATGAACTGTGCAGTCGGAGACACGCAGGGA/ideoxyU/GAGA/ideoxyU/GG/ideoxyU |
| SEQ ID No: 574 | TCTGATGTATGTTGTGCAGTCGGAGACACGCAGGGA/ideoxyU/GAGA/ideoxyU/GG/ideoxyU |
| SEQ ID No: 575 | TCTGCATCTAGCCGTGCAGTCGGAGACACGCAGGGA/ideoxyU/GAGA/ideoxyU/GG/ideoxyU |
| SEQ ID No: 576 | TCAGCACATAACTGTGCAGTCGGAGACACGCAGGGA/ideoxyU/GAGA/ideoxyU/GG/ideoxyU |
| SEQ ID No: 577 | TCATGCTGCTCGGCAGTGCAGTCGGAGACACGCAGGGA/ideoxyU/GAGA/ideoxyU/GG/ideoxyU |
| SEQ ID No: 578 | TCATGATCTGAGGCAGTGCAGTCGGAGACACGCAGGGA/ideoxyU/GAGA/ideoxyU/GG/ideoxyU |
| SEQ ID No: 579 | TCGCCACTGATCAGTGCAGTCGGAGACACGCAGGGA/ideoxyU/GAGA/ideoxyU/GG/ideoxyU |
| SEQ ID No: 580 | TCATGCTCCGCAGCAGTGCAGTCGGAGACACGCAGGGA/ideoxyU/GAGA/ideoxyU/GG/ideoxyU |
| SEQ ID No: 581 | TCATGCCACAGCACTGTGCAGTCGGAGACACGCAGGGA/ideoxyU/GAGA/ideoxyU/GG/ideoxyU |
| SEQ ID No: 582 | TCATATGCCATATGTGTGCAGTCGGAGACACGCAGGGA/ideoxyU/GAGA/ideoxyU/GG/ideoxyU |
| SEQ ID No: 583 | TCTGTGGCGATCTGTGCAGTCGGAGACACGCAGGGA/ideoxyU/GAGA/ideoxyU/GG/ideoxyU |
| SEQ ID No: 584 | TCATCTGAGCCGTGTGCAGTCGGAGACACGCAGGGA/ideoxyU/GAGA/ideoxyU/GG/ideoxyU |
| SEQ ID No: 585 | TCACTATCATCAAGTGCAGTCGGAGACACGCAGGGA/ideoxyU/GAGA/ideoxyU/GG/ideoxyU |
| SEQ ID No: 586 | TCTATGTGCAGTTGTGCAGTCGGAGACACGCAGGGA/ideoxyU/GAGA/ideoxyU/GG/ideoxyU |
| SEQ ID No: 587 | TCAGGATGTATGCGTGCAGTCGGAGACACGCAGGGA/ideoxyU/GAGA/ideoxyU/GG/ideoxyU |
| SEQ ID No: 588 | TCTGCAGCCAGAGATGTGCAGTCGGAGACACGCAGGGA/ideoxyU/GAGA/ideoxyU/GG/ideoxyU |
| SEQ ID No: 589 | TCACATGCACCAGATGTGCAGTCGGAGACACGCAGGGA/ideoxyU/GAGA/ideoxyU/GG/ideoxyU |
| SEQ ID No: 590 | TCGCGAAGCTGCTGTGCAGTCGGAGACACGCAGGGA/ideoxyU/GAGA/ideoxyU/GG/ideoxyU |
| SEQ ID No: 591 | TCTGCTGCCATATGCGTGCAGTCGGAGACACGCAGGGA/ideoxyU/GAGA/ideoxyU/GG/ideoxyU |
| SEQ ID No: 592 | TCAGCGGTGCTGTGTGCAGTCGGAGACACGCAGGGA/ideoxyU/GAGA/ideoxyU/GG/ideoxyU |
| SEQ ID No: 593 | TCACCAGATGCTAGTGCAGTCGGAGACACGCAGGGA/ideoxyU/GAGA/ideoxyU/GG/ideoxyU |
| SEQ ID No: 594 | TCTGTATGCTGAAGTGCAGTCGGAGACACGCAGGGA/ideoxyU/GAGA/ideoxyU/GG/ideoxyU |
| SEQ ID No: 595 | TCTCGCCATCTGCATGTGCAGTCGGAGACACGCAGGGA/ideoxyU/GAGA/ideoxyU/GG/ideoxyU |
| SEQ ID No: 596 | TCTGGATAGATCTGTGCAGTCGGAGACACGCAGGGA/ideoxyU/GAGA/ideoxyU/GG/ideoxyU |
| SEQ ID No: 597 | TCTGAGATGCATGAAGTGCAGTCGGAGACACGCAGGGA/ideoxyU/GAGA/ideoxyU/GG/ideoxyU |
| SEQ ID No: 598 | TCATCACACTTGCATGTGCAGTCGGAGACACGCAGGGA/ideoxyU/GAGA/ideoxyU/GG/ideoxyU |
| SEQ ID No: 599 | TCTGCAGCGAACAGTGCAGTCGGAGACACGCAGGGA/ideoxyU/GAGA/ideoxyU/GG/ideoxyU |
| SEQ ID No: 600 | TCATGCAGGATCTGAGTGCAGTCGGAGACACGCAGGGA/ideoxyU/GAGA/ideoxyU/GG/ideoxyU |
| SEQ ID No: 601 | TCATTATCTGTGTGTGCAGTCGGAGACACGCAGGGA/ideoxyU/GAGA/ideoxyU/GG/ideoxyU |
| SEQ ID No: 602 | TCGCCAGAGCATCGTGCAGTCGGAGACACGCAGGGA/ideoxyU/GAGA/ideoxyU/GG/ideoxyU |
| SEQ ID No: 603 | TCAGATGATGGATCTGTGCAGTCGGAGACACGCAGGGA/ideoxyU/GAGA/ideoxyU/GG/ideoxyU |
| SEQ ID No: 604 | TCATATGATGGTGCAGTGCAGTCGGAGACACGCAGGGA/ideoxyU/GAGA/ideoxyU/GG/ideoxyU |
| SEQ ID No: 605 | TCATTGCTATAGCATGTGCAGTCGGAGACACGCAGGGA/ideoxyU/GAGA/ideoxyU/GG/ideoxyU |
| SEQ ID No: 606 | TCGCCATGTGCAGATGTGCAGTCGGAGACACGCAGGGA/ideoxyU/GAGA/ideoxyU/GG/ideoxyU |
| SEQ ID No: 607 | TCATCTTCATGCAGCGTGCAGTCGGAGACACGCAGGGA/ideoxyU/GAGA/ideoxyU/GG/ideoxyU |
| SEQ ID No: 608 | TCATGCATCTGAATAGTGCAGTCGGAGACACGCAGGGA/ideoxyU/GAGA/ideoxyU/GG/ideoxyU |
| SEQ ID No: 609 | TCGCATTGCGCGAGTGCAGTCGGAGACACGCAGGGA/ideoxyU/GAGA/ideoxyU/GG/ideoxyU |
| SEQ ID No: 610 | TCATGCCATCTGCGCGTGCAGTCGGAGACACGCAGGGA/ideoxyU/GAGA/ideoxyU/GG/ideoxyU |
| SEQ ID No: 611 | TCGATGATTCACTGTGCAGTCGGAGACACGCAGGGA/ideoxyU/GAGA/ideoxyU/GG/ideoxyU |
| SEQ ID No: 612 | TCTAGTGTTGCATGTGCAGTCGGAGACACGCAGGGA/ideoxyU/GAGA/ideoxyU/GG/ideoxyU |
| SEQ ID No: 613 | TCATATCATAATAGTGCAGTCGGAGACACGCAGGGA/ideoxyU/GAGA/ideoxyU/GG/ideoxyU |
| SEQ ID No: 614 | TCGATGCTTCTGAGTGCAGTCGGAGACACGCAGGGA/ideoxyU/GAGA/ideoxyU/GG/ideoxyU |
| SEQ ID No: 615 | TCTCAGATATGAAGTGCAGTCGGAGACACGCAGGGA/ideoxyU/GAGA/ideoxyU/GG/ideoxyU |
| SEQ ID No: 616 | TCTCATAGCTATTGTGCAGTCGGAGACACGCAGGGA/ideoxyU/GAGA/ideoxyU/GG/ideoxyU |
| SEQ ID No: 617 | TCTGCAGCTATCCATGTGCAGTCGGAGACACGCAGGGA/ideoxyU/GAGA/ideoxyU/GG/ideoxyU |
| SEQ ID No: 618 | TCGCAGATATTATGTGCAGTCGGAGACACGCAGGGA/ideoxyU/GAGA/ideoxyU/GG/ideoxyU |
| SEQ ID No: 619 | TCTCAGGATGCATGCGTGCAGTCGGAGACACGCAGGGA/ideoxyU/GAGA/ideoxyU/GG/ideoxyU |
| SEQ ID No: 620 | TCTCCTCTGTGCTGTGCAGTCGGAGACACGCAGGGA/ideoxyU/GAGA/ideoxyU/GG/ideoxyU |
| SEQ ID No: 621 | TCAGAGCGCAGTTGTGCAGTCGGAGACACGCAGGGA/ideoxyU/GAGA/ideoxyU/GG/ideoxyU |
| SEQ ID No: 622 | TCTGTCATCTTGTGTGCAGTCGGAGACACGCAGGGA/ideoxyU/GAGA/ideoxyU/GG/ideoxyU |
| SEQ ID No: 623 | TCTAATGTCATCTGTGCAGTCGGAGACACGCAGGGA/ideoxyU/GAGA/ideoxyU/GG/ideoxyU |
| SEQ ID No: 624 | TCATATGTGAGCCATGTGCAGTCGGAGACACGCAGGGA/ideoxyU/GAGA/ideoxyU/GG/ideoxyU |
| SEQ ID No: 625 | TCAGATGCATCTCTTGTGCAGTCGGAGACACGCAGGGA/ideoxyU/GAGA/ideoxyU/GG/ideoxyU |
| SEQ ID No: 626 | TCACAGCGGTCATGTGCAGTCGGAGACACGCAGGGA/ideoxyU/GAGA/ideoxyU/GG/ideoxyU |
| SEQ ID No: 627 | TCGCGCCAGATGCGTGCAGTCGGAGACACGCAGGGA/ideoxyU/GAGA/ideoxyU/GG/ideoxyU |
| SEQ ID No: 628 | TCATCACAGCTAAGTGCAGTCGGAGACACGCAGGGA/ideoxyU/GAGA/ideoxyU/GG/ideoxyU |
| SEQ ID No: 629 | TCATCTATTGCGAGTGCAGTCGGAGACACGCAGGGA/ideoxyU/GAGA/ideoxyU/GG/ideoxyU |
| SEQ ID No: 630 | TCATTGAGAGAGTGTGCAGTCGGAGACACGCAGGGA/ideoxyU/GAGA/ideoxyU/GG/ideoxyU |
| SEQ ID No: 631 | TCATCATCAGGCAGTGTGCAGTCGGAGACACGCAGGGA/ideoxyU/GAGA/ideoxyU/GG/ideoxyU |
| SEQ ID No: 632 | TCATATGAATAGAGTGCAGTCGGAGACACGCAGGGA/ideoxyU/GAGA/ideoxyU/GG/ideoxyU |
| SEQ ID No: 633 | TCAGTGCGCTCAAGTGCAGTCGGAGACACGCAGGGA/ideoxyU/GAGA/ideoxyU/GG/ideoxyU |
| SEQ ID No: 634 | TCGCCTGATCAGCGTGCAGTCGGAGACACGCAGGGA/ideoxyU/GAGA/ideoxyU/GG/ideoxyU |
| SEQ ID No: 635 | TCATCAACAGAGCGTGCAGTCGGAGACACGCAGGGA/ideoxyU/GAGA/ideoxyU/GG/ideoxyU |
| SEQ ID No: 636 | TCAGATATGTTGAGTGCAGTCGGAGACACGCAGGGA/ideoxyU/GAGA/ideoxyU/GG/ideoxyU |
| SEQ ID No: 637 | TCTGCTGCGCCGCATGTGCAGTCGGAGACACGCAGGGA/ideoxyU/GAGA/ideoxyU/GG/ideoxyU |
| SEQ ID No: 638 | TCGCGCACAGCAAGTGCAGTCGGAGACACGCAGGGA/ideoxyU/GAGA/ideoxyU/GG/ideoxyU |
| SEQ ID No: 639 | TCATGAACGCTATGTGCAGTCGGAGACACGCAGGGA/ideoxyU/GAGA/ideoxyU/GG/ideoxyU |
| SEQ ID No: 640 | TCAGGCTATGTGCGTGCAGTCGGAGACACGCAGGGA/ideoxyU/GAGA/ideoxyU/GG/ideoxyU |
| SEQ ID No: 641 | TCTGACATGAGAAGTGCAGTCGGAGACACGCAGGGA/ideoxyU/GAGA/ideoxyU/GG/ideoxyU |
| SEQ ID No: 642 | TCTCAGCAGATGGCTGTGCAGTCGGAGACACGCAGGGA/ideoxyU/GAGA/ideoxyU/GG/ideoxyU |
| SEQ ID No: 643 | TCTGGCGCTGAGTGTGCAGTCGGAGACACGCAGGGA/ideoxyU/GAGA/ideoxyU/GG/ideoxyU |
| SEQ ID No: 644 | TCTATCAGATGTTGTGCAGTCGGAGACACGCAGGGA/ideoxyU/GAGA/ideoxyU/GG/ideoxyU |
| SEQ ID No: 645 | TCGATGCGGAGCAGTGCAGTCGGAGACACGCAGGGA/ideoxyU/GAGA/ideoxyU/GG/ideoxyU |
| SEQ ID No: 646 | TCATCAAGCGATAGTGCAGTCGGAGACACGCAGGGA/ideoxyU/GAGA/ideoxyU/GG/ideoxyU |
| SEQ ID No: 647 | TCTGCTGAGAAGAGTGCAGTCGGAGACACGCAGGGA/ideoxyU/GAGA/ideoxyU/GG/ideoxyU |
| SEQ ID No: 648 | TCGTGCACACCATGTGCAGTCGGAGACACGCAGGGA/ideoxyU/GAGA/ideoxyU/GG/ideoxyU |
| SEQ ID No: 649 | TCTAAGATGATATGTGCAGTCGGAGACACGCAGGGA/ideoxyU/GAGA/ideoxyU/GG/ideoxyU |
| SEQ ID No: 650 | TCATAGCAGCACCGTGCAGTCGGAGACACGCAGGGA/ideoxyU/GAGA/ideoxyU/GG/ideoxyU |
| SEQ ID No: 651 | TCATAGGCTGACAGTGCAGTCGGAGACACGCAGGGA/ideoxyU/GAGA/ideoxyU/GG/ideoxyU |
| SEQ ID No: 652 | TCATTGTGTGATCATGTGCAGTCGGAGACACGCAGGGA/ideoxyU/GAGA/ideoxyU/GG/ideoxyU |

Also provided are compositions that comprise one or more reagents, supports, template nucleic acids, adapter molecules, primers, and/or intermediary library molecule complexes, prior to, during, and/or subsequent to one or more operations in the library preparation workflows described herein.

Also provided are kits that comprise one or more reagents, supports, template nucleic acids, adapter molecules, and/or primers that can be used to perform one or more operations in the library preparation workflows described herein. In some instances, a kit comprises at least 8, 12, 16, 32, 64, or 96 non-naturally nucleic acid adapter molecules, each selected from Table 8. In some instances, a kit comprises at least 8, 12, 16, 32, 64, or 96 non-naturally nucleic acid adapter molecules, each selected from Table 9. In some instances, a kit comprises at least 8, 12, 16, 32, 64, or 96 non-naturally nucleic acid adapter molecules, each selected from Table 10.

### EXAMPLES

### Example 1: Library Preparation and Sequencing with Methylated Adapters

In methylation sequencing, the number and location of methylated cystosines in adapters can be used to modulate adapter properties and library preparation workflows. For example, with the library preparation schematic in FIG. 8A, the adapters that are ligated to unconverted templates have complementary sequences (e.g., exist as double-stranded molecules). When the library molecules (adapters plus insert) are converted, the adapter sequences will disassociate due to a decrease in complementarity as unmethylated C's are converted to U's. This is illustrated in FIG. 8B. In contrast, the adapters illustrated in FIG. 8C are fully methylated and lack complementarity along at least a portion of their length. When these fully methylated adapters are ligated to an unconverted template, the adapters already exist as partially single-stranded molecules. Alternatively or in addition, adapters can be partially methylated, where less than all of the cytosines are methylated. There are advantages and tradeoffs in using each of these adapter alternatives. In each of these methods, the same adapter sequences may be ligated to each side of the double-stranded insert molecule. This reduces the complexity of library preparation and increases the percentage of successful ligations.

Completely methylated adapters are protected from conversion. That is, they may be ligated to insert molecules and then exposed to bisulfite/EM-seq conversion without changing in sequence. Adapters such as those in FIG. 8C can retain a Y-shaped structure throughout library preparation.

In contrast, the adapter method illustrated in FIG. 8A utilizes adapters that are partially methylated or non-methylated. FIG. 8B exemplifies the use of completely non-methylated adapters with SEQ ID NOs: 9 and 10. In FIG. 8B, there are 14 total unprotected cytosines in the top and bottom strands. After EM-seq, each of these cytosines are converted to uracils, which will increase the likelihood of adapter strand dissociation.

As such, an adapter or adapter strand described herein may comprise any useful number or percentage of unmethylated cytosines, such as to induce disassociation or not to induce disassociation. In some cases, an adapter or adapter strand may be designed to contain any useful number of uracil residues after conversion. In some cases, an adapter or adapter strand may comprise at least or at most a predetermined threshold (or threshold) number or percentage of unmethylated cytosines. In some cases, an adapter or adapter strand may comprise at least or at most a predetermined threshold (or threshold) number or percentage of methylated cytosines. In some cases, an adapter or adapter strand may comprise at least or at most a predetermined threshold (or threshold) number or percentage of uracil residues after conversion. For example, the threshold number of methylated cytosines pre-conversion, unmethylated cytosines pre-conversion, and/or uracil residues post-conversion in an adapter or adapter strand may be at least 1, 2, 3, 4, 5, 6, 7, 8, 9, 10, 11, 12, 13, 14, 15, 16, 17, 18, 19, 20, 25, 30 or more. Alternatively or in addition, the threshold number of methylated cytosines pre-conversion, unmethylated cytosines pre-conversion, and/or uracil residues post-conversion in an adapter or adapter strand may be at most 1, 2, 3, 4, 5, 6, 7, 8, 9, 10, 11, 12, 13, 14, 15, 16, 17, 18, 19, 20, 25, 30 or less. In another example, the threshold percentage of methylated cytosines pre-conversion, unmethylated cytosines pre-conversion, and/or uracil residues post-conversion in an adapter or adapter strand (percentage being #select residues/total residues or % of length) may be at least 1%, 2%, 3%, 4%, 5%, 6%, 7%, 8%, 9%, 10%, 15%, 20%, 25%, 30%, 35%, 40%4, 45%, 50%, 55%, 60%, 65%, 70%, 75%, 80%, 85%, 90%, 95% or more. Alternatively or in addition, the threshold percentage of methylated cytosines pre-conversion, unmethylated cytosines pre-conversion, and/or uracil residues post-conversion in an adapter or adapter strand (percentage being #select residues/total residues or % of length) may be at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or less.

In some cases, an adapter or adapter strand may not comprise a homopolymer sequence greater than 2, 3, 4, 5, 6, 7, 8, 9, 10 or more bases in length.

TABLE 11
Sequencing results with and without methylation-specific adapters

| | | % of | | % of | |
|---|---|---|---|---|---|
| | | reads | | CpG | |
| | | mappable | % of | sites | |
| | | to | dupli- | with | Strand |
| Deamination | | reference | cate | coverage | Asym- |
| Method/adapter | Genome | genome | reads | >10X | metry |
| EM-Seq/NEB | NA12878 | 90.1 | 15.35 | 88.3 | 0.112 |
| EM-seq adapter | | | | | |
| EM-Seq/SEQ | NA12878 | 90.7 | 18.70 | 87.3 | 0.169 |
| ID Nos: 5 and 12 | | | | | |
| EM-Seq/NEB | K562 | 90.5 | 15.36 | 86.9 | 0.119 |
| EM-seq adapter | | | | | |
| EM-Seq/SEQ | K562 | 90.9 | 18.80 | 82.4 | 0.189 |
| ID Nos: 5 and 12 | | | | | |

As detailed in Table 11, the use of methylated adapters for preparation of sequencing libraries did not result in an overall decrease of sequencing quality data. In particular, the percentage of obtained reads that were mappable to the appropriate reference genome did not differ significantly between the two methods. There were some increases in the percentage of duplicate reads and in the strand asymmetry; however, these were not prohibitive. Further experiments demonstrate that additional sets of methylation-specific adapters perform similarly (see Table 12).

TABLE 12
Comparison of sequencing between methylation-specific adapters

| | | | % of | |
|---|---|---|---|---|
| | % of reads | | CpG | |
| | mappable | | sites | |
| | to | % of | with | Strand |
| | reference | duplicate | coverage | Asym- |
| Adapters | genome | reads | >10X | metry |
| NEB EM-Seq adapter | 90.1 | 12.28 | 87.7 | 0.121 |
| SEQ ID Nos: 5 and 12 | 91.2 | 16.84 | 85.3 | 0.175 |
| SEQ ID Nos: 5 and 14 | 91.2 | 15.89 | 85.1 | 0.17 |
| SEQ ID Nos: 6 and 15 | 89.6 | 15.87 | 87.90 | 0.118 |
| SEQ ID Nos: 5 and 18 | 91.2 | 17.19 | 83.50 | 0.185 |

### Example 2: Increased Pass Filter

During ePCR, adapter-template complexes can be covalently bound to a support (e.g., a sequencing bead) at a 5′ end and covalently bound to an affinity tag (e.g., biotin) at the other 5′ end. Any adapter-template molecules that are not covalently attached to supports prior to entry into ePCR will still be available for amplification and hybridization to supports. If these free adapter-template molecules can be prevented from amplifying that will lead to a decrease in polyclonality. Such a decrease in polyclonality can be achieved, if for example, such free adapter-template molecules are degraded such that they cannot provide a substrate for amplification (e.g., by degrading primer binding sites).

Table 13 compares sequencing results from a standard set of adapters (e.g., SEQ ID Nos: 48 and 49, which are used with 3′ biotin) and a set of pre-amplification degradable adapters (e.g., SEQ ID Nos: 60 and 61). As seen below, the overall percentage of beads post ePCR that amplify is somewhat decreased when using SEQ ID Nos: 60 and 61; however, this is offset by the increase in percentage of sequencing reads that pass quality filters when using SEQ ID Nos: 60 and 61. The increase in sequencing quality outweighs the decreased amplification efficiency in cases where quantity of template molecules is not limiting. Indeed, sequencing quality itself is typically the limiting factor on using sequencing data downstream.

TABLE 13
Reduction in polyclonality and subsequent
increase in sequencing quality

| | | % loaded | % of reads that |
|---|---|---|---|
| Experi- | Adapter | beads that are | pass sequence |
| ment | Sequences | positive ( | quality filters |
| 180698 | SEQ ID Nos: | 82.0% | 78% |
| | 48 and 49 | | |
| 180700 | SEQ ID Nos: | 78.0% | 81% |
| | 60 and 61 | | |
| 180706 | SEQ ID Nos: | 85.4% | 76% |
| | 48 and 49 | | |
| 180707 | SEQ ID Nos: | 79.0% | 81% |
| | 60 and 61 | | |
| 120827 | SEQ ID Nos: | 85.3% | 81% |
| | 48 and 49 | | |
| 120825 | SEQ ID Nos: | 78.5% | 83% |
| | 60 and 61 | | |

### NUMBERED EMBODIMENTS

Embodiment 1. A nucleic acid composition, comprising a first strand hybridized to a second strand, wherein:
   a. a biotin is disposed at a 5′ end of the first strand;
   b. the first strand comprises one or more cleavable moieties within 15 nucleotides of the 5′ end of the first strand; and
   c. a phosphate is disposed at a 5′ end of the second strand.
Embodiment 2. The nucleic acid composition of embodiment 1, wherein the first strand and the second strand have complementary sequences.
Embodiment 3. The nucleic acid composition of any one of embodiments 1-2, wherein the first strand comprises one or more cleavable moieties within 12 nucleotides of the 5′ end of the first strand.
Embodiment 4. The nucleic acid composition of any one of embodiments 1-3, wherein the first strand comprises one or more cleavable moieties within 10 nucleotides of the 5′ end of the first strand.
Embodiment 5. The nucleic acid composition of any one of embodiments 1-4, wherein the first strand comprises one or more cleavable moieties within 7 nucleotides of the 5′ end of the first strand.
Embodiment 6. The nucleic acid composition of any one of embodiments 1-5, wherein the first strand comprises the one or more cleavable moieties within 5 nucleotides of the 5′ end of the first strand.
Embodiment 7. The nucleic acid composition of any one of embodiments 1-6, wherein the one or more cleavable moieties are selected from the group consisting of uracils, ribonucleotide residues, spacers, methylated nucleotide residues, and abasic sites.
Embodiment 8. The nucleic acid composition of embodiment 7, wherein the one or more cleavable moieties comprises one or more uracils.
Embodiment 9. The nucleic acid composition of embodiment 8, wherein the one or more uracils comprises 3 or fewer uracils.
Embodiment 10. The nucleic acid composition of any one of embodiments 1-9, wherein a 3′ end of the first strand comprises a protective group.
Embodiment 11. The nucleic acid composition of any one of embodiments 1-10, wherein a 3′ end of the second strand comprises a protective group.
Embodiment 12. The nucleic acid composition of any one of embodiments 10-11, wherein the protective group is protective against exonuclease activity.
Embodiment 13. The nucleic acid composition of embodiment 12, wherein the protective group is a phosphorothioate.
Embodiment 14. The nucleic acid composition of any one of embodiments 1-13, further comprising a double-stranded insert molecule ligated to the 3′ end of the first strand and the 5′ end of the second strand.
Embodiment 15. The nucleic acid composition of embodiment 14, wherein the double-stranded insert molecule comprises a barcode sequence.
Embodiment 16. The nucleic acid composition of any one of embodiments 1-15, further comprising a bead comprising a single-stranded adapter oligonucleotide coupled thereto, wherein the single-stranded adapter oligonucleotide is hybridized to a complex comprising the first strand, the second strand, and the double-stranded insert molecule.
Embodiment 17. The nucleic acid composition of any one of embodiments 1-16, further comprising a streptavidin bound to the biotin.
Embodiment 18. A nucleic acid composition, comprising a first strand hybridized to a second strand, wherein:
   a. the second strand comprises a biotin disposed at the 3′ end;
   b. the second strand comprises one or more cleavable moieties within 10 nucleotides of the 3′ end; and
   c. the second strand comprises a phosphate disposed at the 5′ end.
Embodiment 19. The nucleic acid composition of embodiment 18, wherein:
   a. the one or more cleavable moieties within 10 nucleotides of the 3′ end comprises one cleavable moiety; and
   b. the second strand comprises an additional one or more cleavable moieties within 15 nucleotides of the 5′ end.
Embodiment 20. The nucleic acid composition of any one of embodiments 18-19, wherein the one or more cleavable moieties are selected from the group consisting of uracils, ribonucleotide residues, spacers, methylated nucleotide residues, and abasic sites.
Embodiment 21. The nucleic acid composition of embodiment 20, wherein the one or more cleavable moieties comprises one or more uracils.
Embodiment 22. The nucleic acid composition of embodiment 21, wherein the one or more uracils comprises 2 uracils.
Embodiment 23. The nucleic acid composition of embodiment 21, wherein the one or more uracils comprises 1 uracil.
Embodiment 24. The nucleic acid composition of any one of embodiments 18-23, wherein the first strand has a length of about 60% or less of the length of the second strand.
Embodiment 25. The nucleic acid composition of embodiment 24, wherein the first strand has a length of about 50% or less of the length of the second strand.
Embodiment 26. The nucleic acid composition of embodiment 18, wherein the second strand comprises a uracil disposed within 10 nucleotides of the 5′ end.
Embodiment 27. The nucleic acid composition of embodiment 26, wherein the second strand comprises a uracil disposed within 7 nucleotides of the 5′ end.
Embodiment 28. The nucleic acid composition of embodiment 26, wherein the second strand has a length of about 60% or less of the length of the first strand.
Embodiment 29. The nucleic acid composition of embodiment 28, wherein the second strand has a length of about 50% or less of the length of the first strand.
Embodiment 30. The nucleic acid composition of any one of embodiments 18, 19, 22, 23, 26, or 27, wherein a 3′ end of the first strand comprises a protective group.
Embodiment 31. The nucleic acid composition of embodiment 30, wherein the protective group is protective against exonuclease activity.
Embodiment 32. The nucleic acid composition of embodiment 31, wherein the protective group is a phosphorothioate.
Embodiment 33. The nucleic acid composition of any one of embodiments 18, 19, 22, 23, 26, or 27, further comprising a double-stranded insert molecule ligated to the 3′ end of the first strand and the 5′ end of the second strand.
Embodiment 34. The nucleic acid composition of embodiment 33, further comprising a bead comprising a single-stranded adapter oligonucleotide coupled thereto, wherein the single-stranded adapter oligonucleotide is hybridized to a complex comprising the first strand, the second strand, and the double-stranded insert molecule.
Embodiment 35. The nucleic acid composition of embodiment 34, further comprising a streptavidin bound to the biotin.
Embodiment 36. The nucleic acid composition of embodiment 33, wherein the double-stranded insert molecule comprises a barcode sequence.
Embodiment 37. A composition, comprising: a double-stranded adapter comprising a first sequence selected from any one of SEQ ID Nos: 1-19.
Embodiment 38. The composition of embodiment 37, wherein the double-stranded adapter is coupled to a template molecule at a first end of the template molecule.
Embodiment 39. The composition of embodiment 38, wherein the template molecule is double-stranded.
Embodiment 40. The composition of embodiment 39, wherein the template molecule is further coupled to a double-stranded adapter at a second end of the template molecule, wherein the double-stranded adapter at the second end comprises a sequence selected from any one of SEQ ID Nos: 1-19.
Embodiment 41. The composition of embodiment 40, wherein each double-stranded adapter comprises the same sequence.
Embodiment 42. The composition of any one of embodiments 37-41, wherein the double-stranded adapter comprises a first region that is double stranded and a second region that is single-stranded.
Embodiment 43. The composition of embodiment 42, wherein the second region is an overhang.
Embodiment 44. A nucleic acid composition, comprising:
a single stranded nucleic acid molecule comprising:
a template molecule,
a first sequence disposed at a 5′ end of the template molecule and comprising a first plurality of uracils converted from cytosines, and
a second sequence disposed at a 3′ end of the template molecule and comprising a second plurality of uracils converted from cytosines, and
wherein an unconverted first sequence, which comprises unconverted cytosines corresponding to the first plurality of uracils, and an unconverted second sequence, which comprises unconverted cytosines corresponding to the second plurality of uracils, are reverse complements.
Embodiment 45. The nucleic acid composition of embodiment 44, further comprising a first conversion sequence, comprising (i) a first sequence configured to bind to the first sequence of the single stranded nucleic acid molecule via complementarity.
Embodiment 46. The nucleic acid composition of embodiment 45, wherein the first conversion sequence further comprises (ii) a first overhang sequence linked to the first sequence of the first conversion sequence, the first overhang sequence comprising one or more of a primer-binding sequence, a unique molecular identifying sequence, and a barcode sequence.
Embodiment 47. The nucleic acid composition of any of embodiments 44-46, further comprising a second conversion sequence, comprising (i) a second sequence capable of binding to the second sequence of the single stranded nucleic acid molecule via complementarity.
Embodiment 48. The nucleic acid composition of embodiment 47, wherein the second conversion sequence further comprises (ii) a second overhang sequence linked to the second sequence of the conversion sequence, the second overhang sequence comprising one or more of a primer-binding region, a unique molecular identifying region, and a barcode sequence.
Embodiment 49. The nucleic acid composition of any of embodiments 46 or 48, wherein the barcode sequence is between 9 and 30 nucleotides in length.
Embodiment 50. The nucleic acid composition of embodiment 49, wherein the barcode sequence is between 9 and 11 nucleotides in length.
Embodiment 51. The nucleic acid composition of any of embodiments 44-49, wherein: the first sequence of the single stranded nucleic acid molecule is between 10 and 50 nucleotides in length, and the second sequence of the single stranded nucleic acid molecule is between 10 and 50 nucleotides in length.
Embodiment 52. The nucleic acid composition of embodiment 51, wherein:
the first sequence of the single stranded nucleic acid molecule is between 10 and 30 nucleotides in length, and
the second sequence of the single stranded nucleic acid molecule is between 10 and 30 nucleotides in length.
Embodiment 53. The nucleic acid composition of any one of embodiments 51-52, wherein the first sequence of the single stranded nucleic acid molecule is between 10 and 15 nucleotides in length, and
the second sequence of the single stranded nucleic acid molecule is between 10 and 15 nucleotides in length.
Embodiment 54. The nucleic acid composition of embodiment 51, wherein:
the first sequence of the single stranded nucleic acid molecule is between 20 and 50 nucleotides in length, and
the second sequence of the single stranded nucleic acid molecule is between 20 and 50 nucleotides in length.
Embodiment 55. The nucleic acid composition of any of embodiments 44-54, wherein: the first sequence comprises a first plurality of uracils, and the second sequence comprises a second plurality of uracils.
Embodiment 56. The nucleic acid composition of embodiment 55, wherein the first plurality of uracils is above a threshold number of uracils.
Embodiment 57. The nucleic acid composition of any of embodiments 55-56, wherein the second plurality of uracils is above a threshold number of uracils.
Embodiment 58. The nucleic acid composition of any of embodiments 56-57, wherein the threshold number of uracils is between 2 and 12 uracils.
Embodiment 59. The nucleic acid composition of embodiment 55, wherein:
the first plurality of uracils is at least a percentage of the length of the first sequence; and the second plurality of uracils is at least the percentage of the length of the second sequence.
Embodiment 60. The nucleic acid composition of embodiment 59, wherein the percentage is about 20%.
Embodiment 61. The nucleic acid composition of any one of embodiments 44-60, wherein the first sequence and or second sequence comprises at least one cytosine residue.
Embodiment 62. The nucleic acid composition of any of embodiments 44-61, wherein the first sequence or the second sequence does not comprise a homopolymer sequence.
Embodiment 63. The nucleic acid composition of embodiment 44, wherein the unconverted first sequence is selected from the group of SEQ ID Nos: 1-8, and the unconverted second sequence is selected from the group of SEQ ID Nos: 9-19.
Embodiment 64. A method of processing a nucleic acid molecule, comprising:
   providing a reaction mixture, comprising: a plurality of template molecules; and a plurality of double-stranded adapters, each comprising a first unconverted sequence hybridized to a second unconverted sequence;
   attaching a double-stranded adapter of the plurality of double-stranded adapters to each of a first end and a second end of a subset of template molecules from the plurality of template molecules, thereby providing a plurality of double-stranded template-adapter complexes; and
   exposing the plurality of double-stranded template-adapter complexes to conditions sufficient to convert one or more unmethylated cytosine residues to uracil residues in double-stranded adapters of the plurality of double-stranded template-adapter complexes, thereby providing a plurality of single-stranded template-adapter molecules.
Embodiment 65. The method of embodiment 64, further comprising:
   performing an amplification reaction using the plurality of single-stranded template-adapter molecules and a plurality of additional pair of adapters comprising first additional adapters and second additional adapters, wherein a first additional adapter of the first additional adapters comprises a first cleavable moiety and a first reactive moiety and a second additional adapter of the second additional adapters comprises a second cleavable moiety, thereby providing template-double-adapter molecules.
Embodiment 66. The method of any of embodiments 64-65, wherein:
   a double-stranded adapter of the plurality of double-stranded adapters comprises an overhang region; and
   the attaching of (b) comprises hybridizing the plurality of double-stranded adapters to the plurality of template molecules and performing a ligation reaction.
Embodiment 67. The method of embodiment 66, wherein the overhang region is disposed at a 3′ end of the double-stranded adapter.
Embodiment 68. The method of embodiment 66, wherein the ligation reaction is performed using a ligase.
Embodiment 69. The method of embodiment 66, wherein the ligation reaction is performed using a ligase and a polymerase.
Embodiment 70. The method of any one of embodiments 65-69, wherein the first cleavable moiety and the second cleavable moiety are each selected from the group consisting of uracils, ribonucleotide residues, spacers, methylated nucleotide residues, and abasic sites.
Embodiment 71. The method of any one of embodiments 64-70, wherein at least 75% of the plurality of double-stranded template-adapter complexes are converted into single-stranded template-adapter molecules.
Embodiment 72. The method of embodiment 71, wherein at least 85% of the plurality of double-stranded template-adapter complexes are converted into single-stranded template-adapter molecules.
Embodiment 73. The method of embodiment 72, wherein at least 95% of the plurality of double-stranded template-adapter complexes are converted into single-stranded template-adapter molecules.
Embodiment 74. The method of any one of embodiments 64-73, wherein the template molecules are double-stranded.
Embodiment 75. The method of any one of embodiments 64-74, wherein the exposing of (c) converts first unconverted sequences and second unconverted sequences to first converted sequences and second converted sequences, respectively.
Embodiment 76. The method of embodiment 75, wherein, in the exposing of (c), prior to providing a plurality of single-stranded template-adapter molecules, the first converted sequences dissociate from the second converted sequences.
Embodiment 77. The method of embodiment 65, further comprising sequencing the template-double-adapter molecules.
Embodiment 78. The method of any one of embodiments 64-77, wherein the exposing of (c) comprises bisulfite conversion.
Embodiment 79. The method of any one of embodiments 64-77, wherein the exposing of (c) comprises EM-seq.
Embodiment 80. The method of embodiment 65, wherein the first unconverted sequence is selected from the group of SEQ ID Nos: 1-8, and the second unconverted sequence is selected from the group of SEQ ID Nos: 9-19.
Embodiment 81. A kit, comprising: at least 96 non-naturally occurring nucleic acid adapter molecules, each comprising a different sequence selected from any one of SEQ ID NOs: 77-268.
Embodiment 82. A kit, comprising: at least 96 non-naturally occurring nucleic acid adapter molecules, each comprising a different sequence selected from any one of SEQ ID NOs: 269-460.
Embodiment 83. A kit, comprising: at least 96 non-naturally occurring nucleic acid adapter molecules, each comprising a different sequence selected from any one of SEQ ID NOs: 461-652.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-83. (canceled)

84. A kit comprising a plurality of nucleic acid adapter molecules comprising at least 8 non-naturally occurring nucleic acid adapter molecules, each of the at least 8 non-naturally occurring nucleic acid adapter molecules comprising a different sequence selected from any one of SEQ ID NOs: 77-268.

85. The kit of claim 84, wherein for each of the plurality of nucleic acid adapter molecules at least one thymine residue is replaced by a uracil residue.

86. The kit of claim 85, wherein the at least one thymine residue replaced by the uracil residue is within SEQ ID NO: 49 for each of the at least 8 non-naturally occurring nucleic acid adapter molecules of the plurality of nucleic acid adapter molecules.

87. The kit of claim 84, wherein for each of the plurality of nucleic acid adapter molecules at least two thymine residues are each replaced by a uracil residue.

88. The kit of claim 87, wherein the at least two thymine residues replaced by the uracil residues are within SEQ ID NO: 49 for each of the at least 8 non-naturally occurring nucleic acid adapter molecules of the plurality of nucleic acid adapter molecules.

89. The kit of claim 84, wherein for each of the plurality of nucleic acid adapter molecules a 3′ thymine residue is phosphorothioated.

90. The kit of claim 84, further comprising an additional plurality of nucleic acid adapter molecules comprising at least 8 additional non-naturally occurring nucleic acid adapter molecules, each of the at least 8 additional non-naturally occurring nucleic acid adapter molecules comprising a different sequence selected from any one of SEQ ID NOs: 461-652, wherein each of the additional plurality of nucleic acid adapter molecules has sequence complementarity to a respective non-naturally occurring nucleic acid adapter molecule of the plurality of nucleic acid adapter molecules.

91. The kit of claim 90, wherein each of the additional plurality of nucleic acid adapter molecules further comprises a biotin coupled to a 3′ end thereof.

92. A kit comprising at least 96 non-naturally occurring nucleic acid adapter molecules, each comprising a different sequence selected from any one of SEQ ID NOs: 77-268.

93. The kit of claim 92, wherein in each of the at least 96 non-naturally occurring nucleic acid adapter molecules a thymine residue disposed at a 3′ end of the non-naturally occurring nucleic acid molecule is phosphorothioated.

94. The kit of claim 92, further comprising at least 96 additional non-naturally occurring nucleic acid adapter molecules, each comprising a different sequence selected from any one of SEQ ID NOs: 461-652, wherein each of the at least 96 additional non-naturally occurring nucleic acid adapter molecules has sequence complementarity to a respective non-naturally occurring nucleic acid adapter molecule of the at least 96 non-naturally occurring nucleic acid adapter molecules.

95. The kit of claim 94, wherein each of the at least 96 additional non-naturally occurring nucleic acid adapter molecules further comprises a biotin coupled to a 3′ end thereof.

96. A kit comprising one or more non-naturally occurring nucleic acid adapter molecules each comprising SEQ ID NO: 49.

97. The kit of claim 96, wherein the SEQ ID NO: 49 is located at a 5′ end of each of the one or more non-naturally occurring nucleic acid adapter molecules.

98. The kit of claim 96, wherein the one or more non-naturally occurring nucleic acid adapter molecules each comprises a different sequence selected from any one of SEQ ID NOs: 77-268.

99. The kit of claim 97, wherein in each of the one or more non-naturally occurring nucleic acid adapter molecules a 3′ thymine is phosphorothioated.


SEQUENCE LISTING (657 sequences)

SEQ ID NO: 1 - DNA, 31 bp, synthetic construct
  source 1..31: mol_type = other DNA
tgcacgtagcgtactgcaacggcatgcagat

SEQ ID NO: 2 - DNA, 38 bp, synthetic construct
  source 1..38: mol_type = other DNA
  modified_base 29: mod_base = OTHER; note = 5-methylcytosine
  modified_base 35: mod_base = OTHER; note = 5-methylcytosine
tgcacgtagcgtactgcaacggcatgcactggtacgat

SEQ ID NO: 3 - DNA, 29 bp, synthetic construct
  source 1..29: mol_type = other DNA
aagcagtggtatcgaacgcagtcagcgat

SEQ ID NO: 4 - DNA, 36 bp, synthetic construct
  source 1..36: mol_type = other DNA
  modified_base 27: mod_base = OTHER; note = 5-methylcytosine
  modified_base 33: mod_base = OTHER; note = 5-methylcytosine
aagcagtggtatcgaacgcagtcagcctggtacgat

SEQ ID NO: 5 - DNA, 27 bp, synthetic construct
  source 1..27: mol_type = other DNA
atgacatcgtaggtcagctgcgacgat

SEQ ID NO: 6 - DNA, 39 bp, synthetic construct
  source 1..39: mol_type = other DNA
  modified_base 25: mod_base = OTHER; note = 5-methylcytosine
  modified_base 32: mod_base = OTHER; note = 5-methylcytosine
  modified_base 35: mod_base = OTHER; note = 5-methylcytosine
atgacatcgtaggtcagctgcgaccagagtactgcagat

SEQ ID NO: 7 - DNA, 33 bp, synthetic construct
  source 1..33: mol_type = other DNA
  modified_base 25: mod_base = OTHER; note = 5-methylcytosine
  modified_base 29: mod_base = OTHER; note = 5-methylcytosine
atgacatcgtaggtcagctgcgaccagacagat

SEQ ID NO: 8 - DNA, 43 bp, synthetic construct
  source 1..43: mol_type = other DNA
  modified_base 25: mod_base = OTHER; note = 5-methylcytosine
  modified_base 32: mod_base = OTHER; note = 5-methylcytosine
  modified_base 35: mod_base = OTHER; note = 5-methylcytosine
  modified_base 37: mod_base = OTHER; note = 5-methylcytosine
atgacatcgtaggtcagctgcgaccagagtactgcacgtagat

SEQ ID NO: 9 - DNA, 32 bp, synthetic construct
  source 1..32: mol_type = other DNA
tctgcatgccgttgcagtacgctacgtgcagg

SEQ ID NO: 10 - DNA, 39 bp, synthetic construct
  source 1..39: mol_type = other DNA
  modified_base 2: mod_base = OTHER; note = 5-methylcytosine
  modified_base 6: mod_base = OTHER; note = 5-methylcytosine
  modified_base 7: mod_base = OTHER; note = 5-methylcytosine
tcgtaccagtgcatgccgttgcagtacgctacgtgcagg

SEQ ID NO: 11 - DNA, 37 bp, synthetic construct
  source 1..37: mol_type = other DNA
  modified_base 2: mod_base = OTHER; note = 5-methylcytosine
  modified_base 6: mod_base = OTHER; note = 5-methylcytosine
  modified_base 7: mod_base = OTHER; note = 5-methylcytosine
tcgtaccaggctgactgcgttcgataccactgcttgg

SEQ ID NO: 12 - DNA, 26 bp, synthetic construct
  source 1..26: mol_type = other DNA
tcgtcgcagctgacctacgatgtcat

SEQ ID NO: 13 - DNA, 38 bp, synthetic construct
  source 1..38: mol_type = other DNA
  modified_base 2: mod_base = OTHER; note = 5-methylcytosine
  modified_base 5: mod_base = OTHER; note = 5-methylcytosine
  modified_base 10: mod_base = OTHER; note = 5-methylcytosine
  modified_base 12: mod_base = OTHER; note = 5-methylcytosine
tctgcagtactctggtcgcagctgacctacgatgtcat

SEQ ID NO: 14 - DNA, 28 bp, synthetic construct
  source 1..28: mol_type = other DNA
tcgtcgcagctgacctacgatgtcatgg

SEQ ID NO: 15 - DNA, 40 bp, synthetic construct
  source 1..40: mol_type = other DNA
  modified_base 2: mod_base = OTHER; note = 5-methylcytosine
  modified_base 5: mod_base = OTHER; note = 5-methylcytosine
  modified_base 10: mod_base = OTHER; note = 5-methylcytosine
  modified_base 12: mod_base = OTHER; note = 5-methylcytosine
tctgcagtactctggtcgcagctgacctacgatgtcatgg

SEQ ID NO: 16 - DNA, 34 bp, synthetic construct
  source 1..34: mol_type = other DNA
  modified_base 2: mod_base = OTHER; note = 5-methylcytosine
  modified_base 6: mod_base = OTHER; note = 5-methylcytosine
tctgtctggtcgcagctgacctacgatgtcatgg

SEQ ID NO: 17 - DNA, 44 bp, synthetic construct
  source 1..44: mol_type = other DNA
  modified_base 2: mod_base = OTHER; note = 5-methylcytosine
  modified_base 5: mod_base = OTHER; note = 5-methylcytosine
  modified_base 9: mod_base = OTHER; note = 5-methylcytosine
  modified_base 14: mod_base = OTHER; note = 5-methylcytosine
  modified_base 16: mod_base = OTHER; note = 5-methylcytosine
tctacgtgcagtactctggtcgcagctgacctacgatgtcatgg

SEQ ID NO: 18 - DNA, 65 bp, synthetic construct
  source 1..65: mol_type = other DNA
atcgtcgcagctgacctacgatgtcatggcccccccccccccccccccccccccccccccccccc

SEQ ID NO: 19 - DNA, 39 bp, synthetic construct
  source 1..39: mol_type = other DNA
  modified_base 3: mod_base = OTHER; note = 5-methylcytosine
  modified_base 6: mod_base = OTHER; note = 5-methylcytosine
  modified_base 11: mod_base = OTHER; note = 5-methylcytosine
  modified_base 13: mod_base = OTHER; note = 5-methylcytosine
atctgcagtactctggtcgcagctgacctacgatgtcat

SEQ ID NO: 20 - DNA, 31 bp, synthetic construct
  source 1..31: mol_type = other DNA
  misc_feature 1..2: note = DNA
  misc_feature 3: note = RNA
  misc_feature 4: note = DNA
  misc_feature 5: note = RNA
  misc_feature 6..9: note = DNA
  misc_feature 10: note = RNA
  misc_feature 11..13: note = DNA
  misc_feature 14: note = RNA
  misc_feature 15..16: note = DNA
  misc_feature 17: note = RNA
  misc_feature 18..19: note = DNA
  misc_feature 20: note = RNA
  misc_feature 21..22: note = DNA
  misc_feature 23: note = RNA
  misc_feature 24..26: note = DNA
  misc_feature 27: note = RNA
  misc_feature 28..31: note = DNA
tgtatgtagtgtattgtaatggtatgtagat

SEQ ID NO: 21 - DNA, 38 bp, synthetic construct
  source 1..38: mol_type = other DNA
  modified_base 29: mod_base = OTHER; note = 5-methylcytosine
  modified_base 35: mod_base = OTHER; note = 5-methylcytosine
  misc_feature 1..2: note = DNA
  misc_feature 3: note = RNA
  misc_feature 4: note = DNA
  misc_feature 5: note = RNA
  misc_feature 6..9: note = DNA
  misc_feature 10: note = RNA
  misc_feature 11..13: note = DNA
  misc_feature 14: note = RNA
  misc_feature 15..16: note = DNA
  misc_feature 17: note = RNA
  misc_feature 18..19: note = DNA
  misc_feature 20: note = RNA
  misc_feature 21..22: note = DNA
  misc_feature 23: note = RNA
  misc_feature 24..26: note = DNA
  misc_feature 27: note = RNA
  misc_feature 28..38: note = DNA
tgtatgtagtgtattgtaatggtatgtactggtacgat

SEQ ID NO: 22 - DNA, 29 bp, synthetic construct
  source 1..29: mol_type = other DNA
  misc_feature 1..3: note = DNA
  misc_feature 4: note = RNA
  misc_feature 5..12: note = DNA
  misc_feature 13: note = RNA
  misc_feature 14..16: note = DNA
  misc_feature 17: note = RNA
  misc_feature 18: note = DNA
  misc_feature 19: note = RNA
  misc_feature 20..22: note = DNA
  misc_feature 23: note = RNA
  misc_feature 24..25: note = DNA
  misc_feature 26: note = RNA
  misc_feature 27..29: note = DNA
aagtagtggtattgaatgtagttagtgat

SEQ ID NO: 23 - DNA, 36 bp, synthetic construct
  source 1..36: mol_type = other DNA
  modified_base 27: mod_base = OTHER; note = 5-methylcytosine
  modified_base 33: mod_base = OTHER; note = 5-methylcytosine
  misc_feature 1..3: note = DNA
  misc_feature 4: note = RNA
  misc_feature 5..12: note = DNA
  misc_feature 13: note = RNA
  misc_feature 14..16: note = DNA
  misc_feature 17: note = RNA
  misc_feature 18: note = DNA
  misc_feature 19: note = RNA
  misc_feature 20..22: note = DNA
  misc_feature 23: note = RNA
  misc_feature 24..25: note = DNA
  misc_feature 26: note = RNA
  misc_feature 27..36: note = DNA
aagtagtggtattgaatgtagttagtctggtacgat

SEQ ID NO: 24 - DNA, 27 bp, synthetic construct
  source 1..27: mol_type = other DNA
  misc_feature 1..4: note = DNA
  misc_feature 5: note = RNA
  misc_feature 6..7: note = DNA
  misc_feature 8: note = RNA
  misc_feature 9..14: note = DNA
  misc_feature 15: note = RNA
  misc_feature 16..17: note = DNA
  misc_feature 18: note = RNA
  misc_feature 19..20: note = DNA
  misc_feature 21: note = RNA
  misc_feature 22..23: note = DNA
  misc_feature 24: note = RNA
  misc_feature 25..27: note = DNA
atgatattgtaggttagttgtgatgat

SEQ ID NO: 25 - DNA, 39 bp, synthetic construct
  source 1..39: mol_type = other DNA
  modified_base 25: mod_base = OTHER; note = 5-methylcytosine
  modified_base 32: mod_base = OTHER; note = 5-methylcytosine
  modified_base 35: mod_base = OTHER; note = 5-methylcytosine
  misc_feature 1..4: note = DNA
  misc_feature 5: note = RNA
  misc_feature 6..7: note = DNA
  misc_feature 8: note = RNA
  misc_feature 9..14: note = DNA
  misc_feature 15: note = RNA
  misc_feature 16..17: note = DNA
  misc_feature 18: note = RNA
  misc_feature 19..20: note = DNA
  misc_feature 21: note = RNA
  misc_feature 22..23: note = DNA
  misc_feature 24: note = RNA
  misc_feature 25..39: note = DNA
atgatattgtaggttagttgtgatcagagtactgcagat

SEQ ID NO: 26 - DNA, 33 bp, synthetic construct
  source 1..33: mol_type = other DNA
  modified_base 25: mod_base = OTHER; note = 5-methylcytosine
  modified_base 29: mod_base = OTHER; note = 5-methylcytosine
  misc_feature 1..4: note = DNA
  misc_feature 5: note = RNA
  misc_feature 6..7: note = DNA
  misc_feature 8: note = RNA
  misc_feature 9..14: note = DNA
  misc_feature 15: note = RNA
  misc_feature 16..17: note = DNA
  misc_feature 18: note = RNA
  misc_feature 19..20: note = DNA
  misc_feature 21: note = RNA
  misc_feature 22..23: note = DNA
  misc_feature 24: note = RNA
  misc_feature 25..33: note = DNA
atgatattgtaggttagttgtgatcagacagat

SEQ ID NO: 27 - DNA, 43 bp, synthetic construct
  source 1..43: mol_type = other DNA
  modified_base 25: mod_base = OTHER; note = 5-methylcytosine
  modified_base 32: mod_base = OTHER; note = 5-methylcytosine
  modified_base 35: mod_base = OTHER; note = 5-methylcytosine
  modified_base 37: mod_base = OTHER; note = 5-methylcytosine
  misc_feature 1..4: note = DNA
  misc_feature 5: note = RNA
  misc_feature 6..7: note = DNA
  misc_feature 8: note = RNA
  misc_feature 9..14: note = DNA
  misc_feature 15: note = RNA
  misc_feature 16..17: note = DNA
  misc_feature 18: note = RNA
  misc_feature 19..20: note = DNA
  misc_feature 21: note = RNA
  misc_feature 22..23: note = DNA
  misc_feature 24: note = RNA
  misc_feature 25..43: note = DNA
atgatattgtaggttagttgtgatcagagtactgcacgtagat

SEQ ID NO: 28 - DNA, 32 bp, synthetic construct
  source 1..32: mol_type = other DNA
  misc_feature 1: note = DNA
  misc_feature 2: note = RNA
  misc_feature 3..4: note = DNA
  misc_feature 5: note = RNA
  misc_feature 6..8: note = DNA
  misc_feature 9..10: note = RNA
  misc_feature 11..14: note = DNA
  misc_feature 15: note = RNA
  misc_feature 16..19: note = DNA
  misc_feature 20: note = RNA
  misc_feature 21: note = DNA
  misc_feature 22: note = RNA
  misc_feature 23..24: note = DNA
  misc_feature 25: note = RNA
  misc_feature 26..28: note = DNA
  misc_feature 29: note = RNA
  misc_feature 30..32: note = DNA
tttgtatgttgttgtagtatgttatgtgtagg

SEQ ID NO: 29 - DNA, 39 bp, synthetic construct
  source 1..39: mol_type = other DNA
  modified_base 2: mod_base = OTHER; note = 5-methylcytosine
  modified_base 6: mod_base = OTHER; note = 5-methylcytosine
  modified_base 7: mod_base = OTHER; note = 5-methylcytosine
  misc_feature 1..11: note = DNA
  misc_feature 12: note = RNA
  misc_feature 13..15: note = DNA
  misc_feature 16..17: note = RNA
  misc_feature 18..21: note = DNA
  misc_feature 22: note = RNA
  misc_feature 23..26: note = DNA
  misc_feature 27: note = RNA
  misc_feature 28: note = DNA
  misc_feature 29: note = RNA
  misc_feature 30..31: note = DNA
  misc_feature 32: note = RNA
  misc_feature 33..35: note = DNA
  misc_feature 36: note = RNA
  misc_feature 37..39: note = DNA
tcgtaccagtgtatgttgttgtagtatgttatgtgtagg

SEQ ID NO: 30 - DNA, 37 bp, synthetic construct
  source 1..37: mol_type = other DNA
  modified_base 2: mod_base = OTHER; note = 5-methylcytosine
  modified_base 6: mod_base = OTHER; note = 5-methylcytosine
  modified_base 7: mod_base = OTHER; note = 5-methylcytosine
  misc_feature 1..10: note = DNA
  misc_feature 11: note = RNA
  misc_feature 12..14: note = DNA
  misc_feature 15: note = RNA
  misc_feature 16..17: note = DNA
  misc_feature 18: note = RNA
  misc_feature 19..21: note = DNA
  misc_feature 22: note = RNA
  misc_feature 23..26: note = DNA
  misc_feature 27..28: note = RNA
  misc_feature 29: note = DNA
  misc_feature 30: note = RNA
  misc_feature 31..32: note = DNA
  misc_feature 33: note = RNA
  misc_feature 34..37: note = DNA
tcgtaccaggttgattgtgtttgatattattgtttgg

SEQ ID NO: 31 - DNA, 26 bp, synthetic construct
  source 1..26: mol_type = other DNA
  misc_feature 1: note = DNA
  misc_feature 2: note = RNA
  misc_feature 3..4: note = DNA
  misc_feature 5: note = RNA
  misc_feature 6: note = DNA
  misc_feature 7: note = RNA
  misc_feature 8..9: note = DNA
  misc_feature 10: note = RNA
  misc_feature 11..13: note = DNA
  misc_feature 14..15: note = RNA
  misc_feature 16..17: note = DNA
  misc_feature 18: note = RNA
  misc_feature 19..23: note = DNA
  misc_feature 24: note = RNA
  misc_feature 25..26: note = DNA
ttgttgtagttgatttatgatgttat

SEQ ID NO: 32 - DNA, 38 bp, synthetic construct
  source 1..38: mol_type = other DNA
  modified_base 2: mod_base = OTHER; note = 5-methylcytosine
  modified_base 5: mod_base = OTHER; note = 5-methylcytosine
  modified_base 10: mod_base = OTHER; note = 5-methylcytosine
  modified_base 12: mod_base = OTHER; note = 5-methylcytosine
  misc_feature 1..16: note = DNA
  misc_feature 17: note = RNA
  misc_feature 18: note = DNA
  misc_feature 19: note = RNA
  misc_feature 20..21: note = DNA
  misc_feature 22: note = RNA
  misc_feature 23..25: note = DNA
  misc_feature 26..27: note = RNA
  misc_feature 28..29: note = DNA
  misc_feature 30: note = RNA
  misc_feature 31..35: note = DNA
  misc_feature 36: note = RNA
  misc_feature 37..38: note = DNA
tctgcagtactctggttgtagttgatttatgatgttat

SEQ ID NO: 33 - DNA, 28 bp, synthetic construct
  source 1..28: mol_type = other DNA
  misc_feature 1: note = DNA
  misc_feature 2: note = RNA
  misc_feature 3..4: note = DNA
  misc_feature 5: note = RNA
  misc_feature 6: note = DNA
  misc_feature 7: note = RNA
  misc_feature 8..9: note = DNA
  misc_feature 10: note = RNA
  misc_feature 11..13: note = DNA
  misc_feature 14..15: note = RNA
  misc_feature 16..17: note = DNA
  misc_feature 18: note = RNA
  misc_feature 19..23: note = DNA
  misc_feature 24: note = RNA
  misc_feature 25..28: note = DNA
ttgttgtagttgatttatgatgttatgg

SEQ ID NO: 34 - DNA, 40 bp, synthetic construct
  source 1..40: mol_type = other DNA
  modified_base 2: mod_base = OTHER; note = 5-methylcytosine
  modified_base 5: mod_base = OTHER; note = 5-methylcytosine
  modified_base 10: mod_base = OTHER; note = 5-methylcytosine
  modified_base 12: mod_base = OTHER; note = 5-methylcytosine
  misc_feature 1..16: note = DNA
  misc_feature 17: note = RNA
  misc_feature 18: note = DNA
  misc_feature 19: note = RNA
  misc_feature 20..21: note = DNA
  misc_feature 22: note = RNA
  misc_feature 23..25: note = DNA
  misc_feature 26..27: note = RNA
  misc_feature 28..29: note = DNA
  misc_feature 30: note = RNA
  misc_feature 31..35: note = DNA
  misc_feature 36: note = RNA
  misc_feature 37..40: note = DNA
tctgcagtactctggttgtagttgatttatgatgttatgg

SEQ ID NO: 35 - DNA, 34 bp, synthetic construct
  source 1..34: mol_type = other DNA
  modified_base 2: mod_base = OTHER; note = 5-methylcytosine
  modified_base 6: mod_base = OTHER; note = 5-methylcytosine
  misc_feature 1..10: note = DNA
  misc_feature 11: note = RNA
  misc_feature 12: note = DNA
  misc_feature 13: note = RNA
  misc_feature 14..15: note = DNA
  misc_feature 16: note = RNA
  misc_feature 17..19: note = DNA
  misc_feature 20..21: note = RNA
  misc_feature 22..23: note = DNA
  misc_feature 24: note = RNA
  misc_feature 25..29: note = DNA
  misc_feature 30: note = RNA
  misc_feature 31..34: note = DNA
tctgtctggttgtagttgatttatgatgttatgg

SEQ ID NO: 36 - DNA, 44 bp, synthetic construct
  source 1..44: mol_type = other DNA
  modified_base 2: mod_base = OTHER; note = 5-methylcytosine
  modified_base 5: mod_base = OTHER; note = 5-methylcytosine
  modified_base 9: mod_base = OTHER; note = 5-methylcytosine
  modified_base 14: mod_base = OTHER; note = 5-methylcytosine
  modified_base 16: mod_base = OTHER; note = 5-methylcytosine
  misc_feature 1..20: note = DNA
  misc_feature 21: note = RNA
  misc_feature 22: note = DNA
  misc_feature 23: note = RNA
  misc_feature 24..25: note = DNA
  misc_feature 26: note = RNA
  misc_feature 27..29: note = DNA
  misc_feature 30..31: note = RNA
  misc_feature 32..33: note = DNA
  misc_feature 34: note = RNA
  misc_feature 35..39: note = DNA
  misc_feature 40: note = RNA
  misc_feature 41..44: note = DNA
tctacgtgcagtactctggttgtagttgatttatgatgttatgg

SEQ ID NO: 37 - DNA, 65 bp, synthetic construct
  source 1..65: mol_type = other DNA
  misc_feature 1..2: note = DNA
  misc_feature 3: note = RNA
  misc_feature 4..5: note = DNA
  misc_feature 6: note = RNA
  misc_feature 7: note = DNA
  misc_feature 8: note = RNA
  misc_feature 9..10: note = DNA
  misc_feature 11: note = RNA
  misc_feature 12..14: note = DNA
  misc_feature 15..16: note = RNA
  misc_feature 17..18: note = DNA
  misc_feature 19: note = RNA
  misc_feature 20..24: note = DNA
  misc_feature 25: note = RNA
  misc_feature 26..29: note = DNA
  misc_feature 30..65: note = RNA
attgttgtagttgatttatgatgttatggtttttttttttttttttttttttttttttttttttt

SEQ ID NO: 38 - DNA, 39 bp, synthetic construct
  source 1..39: mol_type = other DNA
  modified_base 3: mod_base = OTHER; note = 5-methylcytosine
  modified_base 6: mod_base = OTHER; note = 5-methylcytosine
  modified_base 11: mod_base = OTHER; note = 5-methylcytosine
  modified_base 13: mod_base = OTHER; note = 5-methylcytosine
  misc_feature 1..17: note = DNA
  misc_feature 18: note = RNA
  misc_feature 19: note = DNA
  misc_feature 20: note = RNA
  misc_feature 21..22: note = DNA
  misc_feature 23: note = RNA
  misc_feature 24..26: note = DNA
  misc_feature 27..28: note = RNA
  misc_feature 29..30: note = DNA
  misc_feature 31: note = RNA
  misc_feature 32..36: note = DNA
  misc_feature 37: note = RNA
  misc_feature 38..39: note = DNA
atctgcagtactctggttgtagttgatttatgatgttat

SEQ ID NO: 39 - DNA, 24 bp, synthetic construct
  source 1..24: mol_type = other DNA
atgatattgtaggttagttgtgat

SEQ ID NO: 40 - DNA, 25 bp, synthetic construct
  source 1..25: mol_type = other DNA
atgatattgtaggttagttgtgatg

SEQ ID NO: 41 - DNA, 24 bp, synthetic construct
  source 1..24: mol_type = other DNA
ataacatcataaatcaactacaac

SEQ ID NO: 42 - DNA, 24 bp, synthetic construct
  source 1..24: mol_type = other DNA
atgtagtgtattgtaatggtatgt

SEQ ID NO: 43 - DNA, 24 bp, synthetic construct
  source 1..24: mol_type = other DNA
acataacatactacaacaacatac

SEQ ID NO: 44 - DNA, 23 bp, synthetic construct
  source 1..23: mol_type = other DNA
tagtggtattgaatgtagttagt

SEQ ID NO: 45 - DNA, 23 bp, synthetic construct
  source 1..23: mol_type = other DNA
caataatatcaaacacaatcaac

SEQ ID NO: 46 - DNA, 48 bp, synthetic construct
  source 1..48: mol_type = other DNA
  modified_base 1: mod_base = OTHER; note = Deoxyuracil
  modified_base 5: mod_base = OTHER; note = Deoxyuracil
  modified_base 7: mod_base = OTHER; note = Deoxyuracil
tccatctcatccctgcgtgtctccgactgcacacatcctgcatgtgat

SEQ ID NO: 47 - DNA, 46 bp, synthetic construct
  source 1..46: mol_type = other DNA
tcacatgcaggatgtgtgcagtcggagacacgcagggatgagatgg

SEQ ID NO: 48 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcacatgcaggatgtgtgcagtcggagacacgcagggatgagatggt

SEQ ID NO: 49 - DNA, 29 bp, synthetic construct
  source 1..29: mol_type = other DNA
atctcatccctgcgtgtctccgactgcac

SEQ ID NO: 50 - DNA, 45 bp, synthetic construct
  source 1..45: mol_type = other DNA
atctcatccctgcgtgtctccgactgcacacatcctgcatgtgat

SEQ ID NO: 51 - DNA, 43 bp, synthetic construct
  source 1..43: mol_type = other DNA
atctcatccctgcgtgtctccgactgcacagttcatctgtgat

SEQ ID NO: 52 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacagatgaactgtgcagtcggagacacgcagggatgagatggt

SEQ ID NO: 53 - DNA, 44 bp, synthetic construct
  source 1..44: mol_type = other DNA
  modified_base 39: mod_base = OTHER; note = Deoxyuracil
  modified_base 44: mod_base = OTHER; note = Deoxyuracil
tcacatgcaggatgtgtgcagtcggagacacgcagggatgagat

SEQ ID NO: 54 - DNA, 26 bp, synthetic construct
  source 1..26: mol_type = other DNA
ccgactgcacacatcctgcatgtgat

SEQ ID NO: 55 - DNA, 42 bp, synthetic construct
  source 1..42: mol_type = other DNA
  modified_base 37: mod_base = OTHER; note = Deoxyuracil
tctgcatctagccgtgcagtcggagacacgcagggatgagat

SEQ ID NO: 56 - DNA, 24 bp, synthetic construct
  source 1..24: mol_type = other DNA
ccgactgcacggctagatgcagat

SEQ ID NO: 57 - DNA, 44 bp, synthetic construct
  source 1..44: mol_type = other DNA
  modified_base 1: mod_base = OTHER; note = Deoxyuracil
  modified_base 10: mod_base = OTHER; note = Deoxyuracil
  modified_base 39: mod_base = OTHER; note = Deoxyuracil
tctgctgaatgcatcgtgcagtcggagacacgcagggatgagat

SEQ ID NO: 58 - DNA, 26 bp, synthetic construct
  source 1..26: mol_type = other DNA
ccgactgcacgatgcattcagcagat

SEQ ID NO: 59 - DNA, 25 bp, synthetic construct
  source 1..25: mol_type = other DNA
  modified_base 6: mod_base = OTHER; note = Deoxyuracil
  modified_base 17: mod_base = OTHER; note = Deoxyuracil
  modified_base 22: mod_base = OTHER; note = Deoxyuracil
tcacatgcaggatgtgtgcagtcgg

SEQ ID NO: 60 - DNA, 45 bp, synthetic construct
  source 1..45: mol_type = other DNA
  modified_base 2: mod_base = OTHER; note = Deoxyuracil
  modified_base 10: mod_base = OTHER; note = Deoxyuracil
  modified_base 16: mod_base = OTHER; note = Deoxyuracil
  modified_base 24: mod_base = OTHER; note = Deoxyuracil
atcaccgactgcccatagagagctgagactgccaaggcacacagg

SEQ ID NO: 61 - DNA, 22 bp, synthetic construct
  source 1..22: mol_type = other DNA
ctctctatgggcagtcggtgat

SEQ ID NO: 62 - DNA, 32 bp, synthetic construct
  source 1..32: mol_type = other DNA
atctcatccctgcgtgtctccgactgcacgat

SEQ ID NO: 63 - DNA, 34 bp, synthetic construct
  source 1..34: mol_type = other DNA
  modified_base 26: mod_base = OTHER; note = Deoxyuracil
  modified_base 31: mod_base = OTHER; note = Deoxyuracil
  modified_base 34: mod_base = OTHER; note = Deoxyuracil
tcgtgcagtcggagacacgcagggatgagatggt

SEQ ID NO: 64 - DNA, 45 bp, synthetic construct; identical to SEQ ID NO: 60
  source 1..45: mol_type = other DNA
  modified_base 10: mod_base = OTHER; note = Deoxyuracil
  modified_base 16: mod_base = OTHER; note = Deoxyuracil
  modified_base 30: mod_base = OTHER; note = Deoxyuracil

SEQ ID NO: 65 - DNA, 46 bp, synthetic construct
  source 1..46: mol_type = other DNA
  misc_feature 45..46: note = Phosphorothioate linkage
  modified_base 10: mod_base = OTHER; note = Deoxyuracil
  modified_base 16: mod_base = OTHER; note = Deoxyuracil
  modified_base 31: mod_base = OTHER; note = Deoxyuracil
atcaccgactgcccatagagagtctgagactgccaaggcacacagg

SEQ ID NO: 66 - DNA, 46 bp, synthetic construct; identical to SEQ ID NO: 65
  source 1..46: mol_type = other DNA
  misc_feature 45..46: note = Phosphorothioate linkage
  misc_feature 1..6: note = DNA
  misc_feature 7: note = RNA
  misc_feature 8..10: note = DNA
  misc_feature 11: note = RNA
  misc_feature 12..19: note = DNA
  misc_feature 20: note = RNA
  misc_feature 21..31: note = DNA
  misc_feature 32: note = RNA
  misc_feature 33..46: note = DNA

SEQ ID NO: 67 - DNA, 45 bp, synthetic construct; identical to SEQ ID NO: 60
  source 1..45: mol_type = other DNA
  misc_feature 44..45: note = Phosphorothioate linkage
  misc_feature 1..6: note = DNA
  misc_feature 7: note = RNA
  misc_feature 8..19: note = DNA
  misc_feature 20: note = RNA
  misc_feature 21..30: note = DNA
  misc_feature 31: note = RNA
  misc_feature 32..45: note = DNA

SEQ ID NO: 68 - DNA, 45 bp, synthetic construct; identical to SEQ ID NO: 60
  source 1..45: mol_type = other DNA
  misc_feature 44..45: note = Phosphorothioate linkage
  misc_feature 1..6: note = DNA
  misc_feature 7: note = RNA
  misc_feature 8..10: note = DNA
  misc_feature 11: note = RNA
  misc_feature 12..19: note = DNA
  misc_feature 20: note = RNA
  misc_feature 21..30: note = DNA
  misc_feature 31: note = RNA
  misc_feature 32..45: note = DNA

SEQ ID NO: 69 - DNA, 45 bp, synthetic construct; identical to SEQ ID NO: 60
  source 1..45: mol_type = other DNA
  modified_base 2: mod_base = OTHER; note = Deoxyuracil
  modified_base 10: mod_base = OTHER; note = Deoxyuracil
  modified_base 16: mod_base = OTHER; note = Deoxyuracil
  modified_base 30: mod_base = OTHER; note = Deoxyuracil

SEQ ID NO: 70 - DNA, 28 bp, synthetic construct
  source 1..28: mol_type = other DNA
  misc_feature 1..3: note = DNA
  misc_feature 4: note = RNA
  misc_feature 5..6: note = DNA
  misc_feature 7: note = RNA
  misc_feature 8..28: note = DNA
tctcagctctctatgggcagtcggtgat

SEQ ID NO: 71 - DNA, 45 bp, synthetic construct
  source 1..45: mol_type = other DNA
  misc_feature 1..3: note = DNA
  misc_feature 4: note = RNA
  misc_feature 5..13: note = DNA
  misc_feature 14: note = RNA
  misc_feature 15..16: note = DNA
  misc_feature 17: note = RNA
  misc_feature 18..22: note = DNA
  misc_feature 23: note = RNA
  misc_feature 24..45: note = DNA
cctgtgtgccttggcagtctcagctctctatgggcagtcggtgat

SEQ ID NO: 72 - DNA, 24 bp, synthetic construct
  source 1..24: mol_type = other DNA
tcctctctatgggcagtcggtgat

SEQ ID NO: 73 - DNA, 24 bp, synthetic construct
  source 1..24: mol_type = other DNA
agctctctatgggcagtcggtgat

SEQ ID NO: 74 - DNA, 30 bp, synthetic construct
  source 1..30: mol_type = other DNA
gactctgacggttccgtgtgtcccctatcc

SEQ ID NO: 75 - DNA, 39 bp, synthetic construct
  source 1..39: mol_type = other DNA
  modified_base 2: mod_base = OTHER; note = Deoxyuracil
  modified_base 10: mod_base = OTHER; note = Deoxyuracil
  modified_base 16: mod_base = OTHER; note = Deoxyuracil
  modified_base 30: mod_base = OTHER; note = Deoxyuracil
atcaccgactgcccatagagagctgagactgccaaggca

SEQ ID NO: 76 - DNA, 23 bp, synthetic construct
  source 1..23: mol_type = other DNA
  misc_feature 22..23: note = Phosphorothioate linkage
ctctctatgggcagtcggtgatt

SEQ ID NO: 77 - DNA, 43 bp, synthetic construct
  source 1..43: mol_type = other DNA
  misc_feature 42..43: note = Phosphorothioate linkage
atctcatccctgcgtgtctccgactgcacagctcgaatgcgat

SEQ ID NO: 78 - DNA, 45 bp, synthetic construct
  source 1..45: mol_type = other DNA
  misc_feature 44..45: note = Phosphorothioate linkage
atctcatccctgcgtgtctccgactgcacatgtgcagccatcgat

SEQ ID NO: 79 - DNA, 45 bp, synthetic construct
  source 1..45: mol_type = other DNA
  misc_feature 44..45: note = Phosphorothioate linkage
atctcatccctgcgtgtctccgactgcacatcacacatgaatgat

SEQ ID NO: 80 - DNA, 43 bp, synthetic construct
  source 1..43: mol_type = other DNA
  misc_feature 42..43: note = Phosphorothioate linkage
atctcatccctgcgtgtctccgactgcactgtgtaggcatgat

SEQ ID NO: 81 - DNA, 43 bp, synthetic construct
  source 1..43: mol_type = other DNA
  misc_feature 42..43: note = Phosphorothioate linkage
atctcatccctgcgtgtctccgactgcacatgtatcctctgat

SEQ ID NO: 82 - DNA, 43 bp, synthetic construct
  source 1..43: mol_type = other DNA
  misc_feature 42..43: note = Phosphorothioate linkage
atctcatccctgcgtgtctccgactgcacatatagcctatgat

SEQ ID NO: 83 - DNA, 43 bp, synthetic construct
  source 1..43: mol_type = other DNA
  misc_feature 42..43: note = Phosphorothioate linkage
atctcatccctgcgtgtctccgactgcacgattcatgctcgat

SEQ ID NO: 84 - DNA, 45 bp, synthetic construct; identical to SEQ ID NO: 50
  source 1..45: mol_type = other DNA
  misc_feature 44..45: note = Phosphorothioate linkage

SEQ ID NO: 85 - DNA, 45 bp, synthetic construct
  source 1..45: mol_type = other DNA
  misc_feature 44..45: note = Phosphorothioate linkage
atctcatccctgcgtgtctccgactgcacgcgcatcctgcatgat

SEQ ID NO: 86 - DNA, 43 bp, synthetic construct
  source 1..43: mol_type = other DNA
  misc_feature 42..43: note = Phosphorothioate linkage
atctcatccctgcgtgtctccgactgcacactgcacgaatgat

SEQ ID NO: 87 - DNA, 43 bp, synthetic construct
  source 1..43: mol_type = other DNA
  misc_feature 42..43: note = Phosphorothioate linkage
atctcatccctgcgtgtctccgactgcacacgcactgccagat

SEQ ID NO: 88 - DNA, 43 bp, synthetic construct
  source 1..43: mol_type = other DNA
  misc_feature 42..43: note = Phosphorothioate linkage
atctcatccctgcgtgtctccgactgcactgccatagcacgat

SEQ ID NO: 89 - DNA, 43 bp, synthetic construct
  source 1..43: mol_type = other DNA
  misc_feature 42..43: note = Phosphorothioate linkage
atctcatccctgcgtgtctccgactgcacagttgtgctgtgat

SEQ ID NO: 90 - DNA, 45 bp, synthetic construct
  source 1..45: mol_type = other DNA
  misc_feature 44..45: note = Phosphorothioate linkage
atctcatccctgcgtgtctccgactgcacatggcgcgatcatgat

SEQ ID NO: 91 - DNA, 43 bp, synthetic construct
  source 1..43: mol_type = other DNA
  misc_feature 42..43: note = Phosphorothioate linkage
atctcatccctgcgtgtctccgactgcacttagatatcatgat

SEQ ID NO: 92 - DNA, 45 bp, synthetic construct
  source 1..45: mol_type = other DNA
  misc_feature 44..45: note = Phosphorothioate linkage
atctcatccctgcgtgtctccgactgcacatcctgtgcgcatgat

SEQ ID NO: 93 - DNA, 43 bp, synthetic construct
  source 1..43: mol_type = other DNA
  misc_feature 42..43: note = Phosphorothioate linkage
atctcatccctgcgtgtctccgactgcacacgtggcacatgat

SEQ ID NO: 94 - DNA, 43 bp, synthetic construct
  source 1..43: mol_type = other DNA
  misc_feature 42..43: note = Phosphorothioate linkage
atctcatccctgcgtgtctccgactgcacatgcgtcctgtgat

SEQ ID NO: 95 - DNA, 45 bp, synthetic construct
  source 1..45: mol_type = other DNA
  misc_feature 44..45: note = Phosphorothioate linkage
atctcatccctgcgtgtctccgactgcacaatgctctgcatagat

SEQ ID NO: 96 - DNA, 43 bp, synthetic construct
  source 1..43: mol_type = other DNA
  misc_feature 42..43: note = Phosphorothioate linkage
atctcatccctgcgtgtctccgactgcactacattgcacagat

SEQ ID NO: 97 - DNA, 43 bp, synthetic construct
  source 1..43: mol_type = other DNA
  misc_feature 42..43: note = Phosphorothioate linkage
atctcatccctgcgtgtctccgactgcacatagcgagccagat

SEQ ID NO: 98 - DNA, 45 bp, synthetic construct
  source 1..45: mol_type = other DNA
  misc_feature 44..45: note = Phosphorothioate linkage
atctcatccctgcgtgtctccgactgcacggagtgcatgcatgat

SEQ ID NO: 99 - DNA, 43 bp, synthetic construct
  source 1..43: mol_type = other DNA
  misc_feature 42..43: note = Phosphorothioate linkage
atctcatccctgcgtgtctccgactgcacatggagatactgat

SEQ ID NO: 100 - DNA, 43 bp, synthetic construct
  source 1..43: mol_type = other DNA
  misc_feature 42..43: note = Phosphorothioate linkage
atctcatccctgcgtgtctccgactgcactgagcctgtcagat

SEQ ID NO: 101 - DNA, 43 bp, synthetic construct
  source 1..43: mol_type = other DNA
  misc_feature 42..43: note = Phosphorothioate linkage
atctcatccctgcgtgtctccgactgcactcgagattgatgat

SEQ ID NO: 102 - DNA, 45 bp, synthetic construct
  source 1..45: mol_type = other DNA
  misc_feature 44..45: note = Phosphorothioate linkage
atctcatccctgcgtgtctccgactgcacatccatctcatctgat

SEQ ID NO: 103 - DNA, 43 bp, synthetic construct
  source 1..43: mol_type = other DNA
  misc_feature 42..43: note = Phosphorothioate linkage
atctcatccctgcgtgtctccgactgcacactgtcagccagat

SEQ ID NO: 104 - DNA, 43 bp, synthetic construct
  source 1..43: mol_type = other DNA
  misc_feature 42..43: note = Phosphorothioate linkage
atctcatccctgcgtgtctccgactgcacagacttgctgcgat

SEQ ID NO: 105 - DNA, 43 bp, synthetic construct
  source 1..43: mol_type = other DNA
  misc_feature 42..43: note = Phosphorothioate linkage
atctcatccctgcgtgtctccgactgcacatatagcaggagat

SEQ ID NO: 106 - DNA, 43 bp, synthetic construct
  source 1..43: mol_type = other DNA
  misc_feature 42..43: note = Phosphorothioate linkage
atctcatccctgcgtgtctccgactgcacatgtctaagatgat

SEQ ID NO: 107 - DNA, 45 bp, synthetic construct
  source 1..45: mol_type = other DNA
  misc_feature 44..45: note = Phosphorothioate linkage
atctcatccctgcgtgtctccgactgcacatcgcagctgaatgat

SEQ ID NO: 108 - DNA, 43 bp, synthetic construct
  source 1..43: mol_type = other DNA
  misc_feature 42..43: note = Phosphorothioate linkage
atctcatccctgcgtgtctccgactgcactctgtattgcagat

SEQ ID NO: 109 - DNA, 43 bp, synthetic construct
  source 1..43: mol_type = other DNA
  misc_feature 42..43: note = Phosphorothioate linkage
atctcatccctgcgtgtctccgactgcacatcatcgattcgat

SEQ ID NO: 110 - DNA, 43 bp, synthetic construct
  source 1..43: mol_type = other DNA
  misc_feature 42..43: note = Phosphorothioate linkage
atctcatccctgcgtgtctccgactgcactgtgaatatgcgat

SEQ ID NO: 111 - DNA, 43 bp, synthetic construct
  source 1..43: mol_type = other DNA
  misc_feature 42..43: note = Phosphorothioate linkage
atctcatccctgcgtgtctccgactgcactgaatgatctcgat

SEQ ID NO: 112 - DNA, 45 bp, synthetic construct
  source 1..45: mol_type = other DNA
  misc_feature 44..45: note = Phosphorothioate linkage
atctcatccctgcgtgtctccgactgcacagaagctgcatgagat

SEQ ID NO: 113 - DNA, 43 bp, synthetic construct
  source 1..43: mol_type = other DNA
  misc_feature 42..43: note = Phosphorothioate linkage
atctcatccctgcgtgtctccgactgcactagattgcgcagat

SEQ ID NO: 114 - DNA, 43 bp, synthetic construct
  source 1..43: mol_type = other DNA
  misc_feature 42..43: note = Phosphorothioate linkage
atctcatccctgcgtgtctccgactgcacgcatcctcacagat

SEQ ID NO: 115 - DNA, 43 bp, synthetic construct
  source 1..43: mol_type = other DNA
  misc_feature 42..43: note = Phosphorothioate linkage
atctcatccctgcgtgtctccgactgcacacaacatatcagat

SEQ ID NO: 116 - DNA, 45 bp, synthetic construct
  source 1..45: mol_type = other DNA
  misc_feature 44..45: note = Phosphorothioate linkage
atctcatccctgcgtgtctccgactgcacatcctgcatcgcagat

SEQ ID NO: 117 - DNA, 45 bp, synthetic construct
  source 1..45: mol_type = other DNA
  misc_feature 44..45: note = Phosphorothioate linkage
atctcatccctgcgtgtctccgactgcacacatcagctcaatgat

SEQ ID NO: 118 - DNA, 45 bp, synthetic construct
  source 1..45: mol_type = other DNA
  misc_feature 44..45: note = Phosphorothioate linkage
atctcatccctgcgtgtctccgactgcacatgcatataatgcgat

SEQ ID NO: 119 - DNA, 45 bp, synthetic construct
  source 1..45: mol_type = other DNA
  misc_feature 44..45: note = Phosphorothioate linkage
atctcatccctgcgtgtctccgactgcactcaatgcatcagcgat

SEQ ID NO: 120 - DNA, 43 bp, synthetic construct
  source 1..43: mol_type = other DNA
  misc_feature 42..43: note = Phosphorothioate linkage
atctcatccctgcgtgtctccgactgcacaatgtgtgctagat

SEQ ID NO: 121 - DNA, 45 bp, synthetic construct
  source 1..45: mol_type = other DNA
  misc_feature 44..45: note = Phosphorothioate linkage
atctcatccctgcgtgtctccgactgcactcagcctcatgctgat

SEQ ID NO: 122 - DNA, 45 bp, synthetic construct
  source 1..45: mol_type = other DNA
  misc_feature 44..45: note = Phosphorothioate linkage
atctcatccctgcgtgtctccgactgcactctcgcatgcaatgat

SEQ ID NO: 123 - DNA, 43 bp, synthetic construct
  source 1..43: mol_type = other DNA
  misc_feature 42..43: note = Phosphorothioate linkage
atctcatccctgcgtgtctccgactgcactagcagccagcgat

SEQ ID NO: 124 - DNA, 43 bp, synthetic construct
  source 1..43: mol_type = other DNA
  misc_feature 42..43: note = Phosphorothioate linkage
atctcatccctgcgtgtctccgactgcactgccagactgtgat

SEQ ID NO: 125 - DNA, 43 bp, synthetic construct
  source 1..43: mol_type = other DNA
  misc_feature 42..43: note = Phosphorothioate linkage
atctcatccctgcgtgtctccgactgcacgcactgctcctgat

SEQ ID NO: 126 - DNA, 45 bp, synthetic construct
  source 1..45: mol_type = other DNA
  misc_feature 44..45: note = Phosphorothioate linkage
atctcatccctgcgtgtctccgactgcacacatggcagcacagat

SEQ ID NO: 127 - DNA, 43 bp, synthetic construct
  source 1..43: mol_type = other DNA
  misc_feature 42..43: note = Phosphorothioate linkage
atctcatccctgcgtgtctccgactgcacacttgcagatcgat

SEQ ID NO: 128 - DNA, 45 bp, synthetic construct
  source 1..45: mol_type = other DNA
  misc_feature 44..45: note = Phosphorothioate linkage
atctcatccctgcgtgtctccgactgcactatgccacagcatgat

SEQ ID NO: 129 - DNA, 45 bp, synthetic construct
  source 1..45: mol_type = other DNA
  misc_feature 44..45: note = Phosphorothioate linkage
atctcatccctgcgtgtctccgactgcacaacatcagcatgagat

SEQ ID NO: 130 - DNA, 45 bp, synthetic construct
  source 1..45: mol_type = other DNA
  misc_feature 44..45: note = Phosphorothioate linkage
atctcatccctgcgtgtctccgactgcactgcagtgattcatgat

SEQ ID NO: 131 - DNA, 45 bp, synthetic construct
  source 1..45: mol_type = other DNA
  misc_feature 44..45: note = Phosphorothioate linkage
atctcatccctgcgtgtctccgactgcacagcacctgcatcagat

SEQ ID NO: 132 - DNA, 43 bp, synthetic construct
  source 1..43: mol_type = other DNA
  misc_feature 42..43: note = Phosphorothioate linkage
atctcatccctgcgtgtctccgactgcacttatgctatcagat

SEQ ID NO: 133 - DNA, 45 bp, synthetic construct
  source 1..45: mol_type = other DNA
  misc_feature 44..45: note = Phosphorothioate linkage
atctcatccctgcgtgtctccgactgcacaatgcgtgctgcagat

SEQ ID NO: 134 - DNA, 45 bp, synthetic construct
  source 1..45: mol_type = other DNA
  misc_feature 44..45: note = Phosphorothioate linkage
atctcatccctgcgtgtctccgactgcacatctcagtgcaatgat

SEQ ID NO: 135 - DNA, 45 bp, synthetic construct
  source 1..45: mol_type = other DNA
  misc_feature 44..45: note = Phosphorothioate linkage
atctcatccctgcgtgtctccgactgcacatgtgcttcgcatgat

SEQ ID NO: 136 - DNA, 45 bp, synthetic construct
  source 1..45: mol_type = other DNA
  misc_feature 44..45: note = Phosphorothioate linkage
atctcatccctgcgtgtctccgactgcacatgagtgcagccagat

SEQ ID NO: 137 - DNA, 45 bp, synthetic construct
  source 1..45: mol_type = other DNA
  misc_feature 44..45: note = Phosphorothioate linkage
atctcatccctgcgtgtctccgactgcactcaagcatgtgctgat

SEQ ID NO: 138 - DNA, 43 bp, synthetic construct
  source 1..43: mol_type = other DNA
  misc_feature 42..43: note = Phosphorothioate linkage
atctcatccctgcgtgtctccgactgcacacagtcaatgtgat

SEQ ID NO: 139 - DNA, 43 bp, synthetic construct
  source 1..43: mol_type = other DNA
  misc_feature 42..43: note = Phosphorothioate linkage
atctcatccctgcgtgtctccgactgcacacacttgatgcgat

SEQ ID NO: 140 - DNA, 43 bp, synthetic construct
  source 1..43: mol_type = other DNA
  misc_feature 42..43: note = Phosphorothioate linkage
atctcatccctgcgtgtctccgactgcacatagagcctcagat

SEQ ID NO: 141 - DNA, 43 bp, synthetic construct
  source 1..43: mol_type = other DNA
  misc_feature 42..43: note = Phosphorothioate linkage
atctcatccctgcgtgtctccgactgcacttgtgtcatgagat

SEQ ID NO: 142 - DNA, 43 bp, synthetic construct
  source 1..43: mol_type = other DNA
  misc_feature 42..43: note = Phosphorothioate linkage
atctcatccctgcgtgtctccgactgcacaatgcactcgagat

SEQ ID NO: 143 - DNA, 43 bp, synthetic construct
  source 1..43: mol_type = other DNA
  misc_feature 42..43: note = Phosphorothioate linkage
atctcatccctgcgtgtctccgactgcacttatgagcgctgat

SEQ ID NO: 144 - DNA, 43 bp, synthetic construct
  source 1..43: mol_type = other DNA
  misc_feature 42..43: note = Phosphorothioate linkage
atctcatccctgcgtgtctccgactgcacgtcattgcacagat

SEQ ID NO: 145 - DNA, 43 bp, synthetic construct
  source 1..43: mol_type = other DNA
  misc_feature 42..43: note = Phosphorothioate linkage
atctcatccctgcgtgtctccgactgcacatcactgcaacgat

SEQ ID NO: 146 - DNA, 45 bp, synthetic construct
  source 1..45: mol_type = other DNA
  misc_feature 44..45: note = Phosphorothioate linkage
atctcatccctgcgtgtctccgactgcacttgcatgcgatgcgat

SEQ ID NO: 147 - DNA, 43 bp, synthetic construct
  source 1..43: mol_type = other DNA
  misc_feature 42..43: note = Phosphorothioate linkage
atctcatccctgcgtgtctccgactgcacgtgcgcaagcagat

SEQ ID NO: 148 - DNA, 43 bp, synthetic construct
  source 1..43: mol_type = other DNA
  misc_feature 42..43: note = Phosphorothioate linkage
atctcatccctgcgtgtctccgactgcactatctcataatgat

SEQ ID NO: 149 - DNA, 45 bp, synthetic construct
  source 1..45: mol_type = other DNA
  misc_feature 44..45: note = Phosphorothioate linkage
atctcatccctgcgtgtctccgactgcacatggctatgcactgat

SEQ ID NO: 150 - DNA, 43 bp, synthetic construct
  source 1..43: mol_type = other DNA
  misc_feature 42..43: note = Phosphorothioate linkage
atctcatccctgcgtgtctccgactgcactatgaatgagcgat

SEQ ID NO: 151 - DNA, 43 bp, synthetic construct
  source 1..43: mol_type = other DNA
  misc_feature 42..43: note = Phosphorothioate linkage
atctcatccctgcgtgtctccgactgcactatgcaccatcgat

SEQ ID NO: 152 - DNA, 43 bp, synthetic construct
  source 1..43: mol_type = other DNA
  misc_feature 42..43: note = Phosphorothioate linkage
atctcatccctgcgtgtctccgactgcacacaatgtgcgcgat

SEQ ID NO: 153 - DNA, 43 bp, synthetic construct
  source 1..43: mol_type = other DNA
  misc_feature 42..43: note = Phosphorothioate linkage
atctcatccctgcgtgtctccgactgcacaatgactatctgat

SEQ ID NO: 154 - DNA, 43 bp, synthetic construct
  source 1..43: mol_type = other DNA
  misc_feature 42..43: note = Phosphorothioate linkage
atctcatccctgcgtgtctccgactgcacagccgctgctcgat

SEQ ID NO: 155 - DNA, 43 bp, synthetic construct
  source 1..43: mol_type = other DNA
  misc_feature 42..43: note = Phosphorothioate linkage
atctcatccctgcgtgtctccgactgcacgcgcgcagaatgat

SEQ ID NO: 156 - DNA, 43 bp, synthetic construct
  source 1..43: mol_type = other DNA
  misc_feature 42..43: note = Phosphorothioate linkage
atctcatccctgcgtgtctccgactgcacagcctcagcgtgat

SEQ ID NO: 157 - DNA, 43 bp, synthetic construct
  source 1..43: mol_type = other DNA
  misc_feature 42..43: note = Phosphorothioate linkage
atctcatccctgcgtgtctccgactgcacttgatcatacagat

SEQ ID NO: 158 - DNA, 45 bp, synthetic construct
  source 1..45: mol_type = other DNA
  misc_feature 44..45: note = Phosphorothioate linkage
atctcatccctgcgtgtctccgactgcactctgctgtgcaatgat

SEQ ID NO: 159 - DNA, 43 bp, synthetic construct
  source 1..43: mol_type = other DNA
  misc_feature 42..43: note = Phosphorothioate linkage
atctcatccctgcgtgtctccgactgcacagtgtattgctgat

SEQ ID NO: 160 - DNA, 45 bp, synthetic construct
  source 1..45: mol_type = other DNA
  misc_feature 44..45: note = Phosphorothioate linkage
atctcatccctgcgtgtctccgactgcactgtgcatctgcctgat

SEQ ID NO: 161 - DNA, 43 bp, synthetic construct
  source 1..43: mol_type = other DNA
  misc_feature 42..43: note = Phosphorothioate linkage
atctcatccctgcgtgtctccgactgcactctatgttgctgat

SEQ ID NO: 162 - DNA, 43 bp, synthetic construct
  source 1..43: mol_type = other DNA
  misc_feature 42..43: note = Phosphorothioate linkage
atctcatccctgcgtgtctccgactgcacaactatctgcagat

SEQ ID NO: 163 - DNA, 45 bp, synthetic construct
  source 1..45: mol_type = other DNA
  misc_feature 44..45: note = Phosphorothioate linkage
atctcatccctgcgtgtctccgactgcacagatctcatgaatgat

SEQ ID NO: 164 - DNA, 43 bp, synthetic construct
  source 1..43: mol_type = other DNA
  misc_feature 42..43: note = Phosphorothioate linkage
atctcatccctgcgtgtctccgactgcactatcatccagtgat

SEQ ID NO: 165 - DNA, 43 bp, synthetic construct
  source 1..43: mol_type = other DNA
  misc_feature 42..43: note = Phosphorothioate linkage
atctcatccctgcgtgtctccgactgcactgctacaagcagat

SEQ ID NO: 166 - DNA, 43 bp, synthetic construct
  source 1..43: mol_type = other DNA
  misc_feature 42..43: note = Phosphorothioate linkage
atctcatccctgcgtgtctccgactgcacaatatgcacgtgat

SEQ ID NO: 167 - DNA, 43 bp, synthetic construct
  source 1..43: mol_type = other DNA
  misc_feature 42..43: note = Phosphorothioate linkage
atctcatccctgcgtgtctccgactgcacacttctgcgatgat

SEQ ID NO: 168 - DNA, 43 bp, synthetic construct
  source 1..43: mol_type = other DNA
  misc_feature 42..43: note = Phosphorothioate linkage
atctcatccctgcgtgtctccgactgcacaagcatatctagat

SEQ ID NO: 169 - DNA, 43 bp, synthetic construct
  source 1..43: mol_type = other DNA
  misc_feature 42..43: note = Phosphorothioate linkage
atctcatccctgcgtgtctccgactgcacaatgacagctcgat

SEQ ID NO: 170 - DNA, 45 bp, synthetic construct
  source 1..45: mol_type = other DNA
  misc_feature 44..45: note = Phosphorothioate linkage
atctcatccctgcgtgtctccgactgcactgatgagcttgatgat

SEQ ID NO: 171 - DNA, 43 bp, synthetic construct
  source 1..43: mol_type = other DNA
  misc_feature 42..43: note = Phosphorothioate linkage
atctcatccctgcgtgtctccgactgcacatatgacctgagat

SEQ ID NO: 172 - DNA, 43 bp, synthetic construct
  source 1..43: mol_type = other DNA
  misc_feature 42..43: note = Phosphorothioate linkage
atctcatccctgcgtgtctccgactgcacagccgatatcagat

SEQ ID NO: 173 - DNA, 43 bp, synthetic construct
  source 1..43: mol_type = other DNA
  misc_feature 42..43: note = Phosphorothioate linkage
atctcatccctgcgtgtctccgactgcacagtcagttgcagat

SEQ ID NO: 174 - DNA, 43 bp, synthetic construct
  source 1..43: mol_type = other DNA
  misc_feature 42..43: note = Phosphorothioate linkage
atctcatccctgcgtgtctccgactgcacaacagatgtatgat

SEQ ID NO: 175 - DNA, 45 bp, synthetic construct
  source 1..45: mol_type = other DNA
  misc_feature 42..43: note = Phosphorothioate linkage
atctcatccctgcgtgtctccgactgcactcatctgcgcaatgat

SEQ ID NO: 176 - DNA, 43 bp, synthetic construct
  source 1..43: mol_type = other DNA
  misc_feature 42..43: note = Phosphorothioate linkage
atctcatccctgcgtgtctccgactgcacggatcatgcgtgat

SEQ ID NO: 177 - DNA, 45 bp, synthetic construct
  source 1..45: mol_type = other DNA
  misc_feature 44..45: note = Phosphorothioate linkage
atctcatccctgcgtgtctccgactgcactattgcatgctctgat

SEQ ID NO: 178 - DNA, 43 bp, synthetic construct
  source 1..43: mol_type = other DNA
  misc_feature 42..43: note = Phosphorothioate linkage
atctcatccctgcgtgtctccgactgcactacctgatgcagat

SEQ ID NO: 179 - DNA, 43 bp, synthetic construct
  source 1..43: mol_type = other DNA
  misc_feature 42..43: note = Phosphorothioate linkage
atctcatccctgcgtgtctccgactgcacatataatcacagat

SEQ ID NO: 180 - DNA, 45 bp, synthetic construct
  source 1..45: mol_type = other DNA
  misc_feature 44..45: note = Phosphorothioate linkage
atctcatccctgcgtgtctccgactgcacatgcagcgctaatgat

SEQ ID NO: 181 - DNA, 45 bp, synthetic construct
  source 1..45: mol_type = other DNA
  misc_feature 44..45: note = Phosphorothioate linkage
atctcatccctgcgtgtctccgactgcacatggcgcagtgctgat

SEQ ID NO: 182 - DNA, 43 bp, synthetic construct
  source 1..43: mol_type = other DNA
  misc_feature 42..43: note = Phosphorothioate linkage
atctcatccctgcgtgtctccgactgcactgagaatgtgtgat

SEQ ID NO: 183 - DNA, 43 bp, synthetic construct
  source 1..43: mol_type = other DNA
  misc_feature 42..43: note = Phosphorothioate linkage
atctcatccctgcgtgtctccgactgcacatgcatggtacgat

SEQ ID NO: 184 - DNA, 45 bp, synthetic construct
  source 1..45: mol_type = other DNA
  misc_feature 44..45: note = Phosphorothioate linkage
atctcatccctgcgtgtctccgactgcacatgcatgcgaggagat

SEQ ID NO: 185 - DNA, 43 bp, synthetic construct
  source 1..43: mol_type = other DNA
  misc_feature 42..43: note = Phosphorothioate linkage
atctcatccctgcgtgtctccgactgcacaatctgcatacgat

SEQ ID NO: 186 - DNA, 45 bp, synthetic construct
  source 1..45: mol_type = other DNA
  misc_feature 44..45: note = Phosphorothioate linkage
atctcatccctgcgtgtctccgactgcacagcatgagcgccagat

SEQ ID NO: 187 - DNA, 43 bp, synthetic construct
  source 1..43: mol_type = other DNA
  misc_feature 42..43: note = Phosphorothioate linkage
atctcatccctgcgtgtctccgactgcacttatctgatctgat

SEQ ID NO: 188 - DNA, 45 bp, synthetic construct
  source 1..45: mol_type = other DNA
  misc_feature 44..45: note = Phosphorothioate linkage
atctcatccctgcgtgtctccgactgcacatccagcgcatgtgat

SEQ ID NO: 189 - DNA, 43 bp, synthetic construct; identical to SEQ ID NO: 51
  source 1..43: mol_type = other DNA
  misc_feature 42..43: note = Phosphorothioate linkage

SEQ ID NO: 190 - DNA, 43 bp, synthetic construct
  source 1..43: mol_type = other DNA
  misc_feature 42..43: note = Phosphorothioate linkage
atctcatccctgcgtgtctccgactgcacaacatacatcagat

SEQ ID NO: 191 - DNA, 43 bp, synthetic construct
  source 1..43: mol_type = other DNA
  misc_feature 42..43: note = Phosphorothioate linkage
atctcatccctgcgtgtctccgactgcacggctagatgcagat

SEQ ID NO: 192 - DNA, 43 bp, synthetic construct
  source 1..43: mol_type = other DNA
  misc_feature 42..43: note = Phosphorothioate linkage
atctcatccctgcgtgtctccgactgcacagttatgtgctgat

SEQ ID NO: 193 - DNA, 45 bp, synthetic construct
  source 1..45: mol_type = other DNA
  misc_feature 44..45: note = Phosphorothioate linkage
atctcatccctgcgtgtctccgactgcactgccgagcagcatgat

SEQ ID NO: 194 - DNA, 45 bp, synthetic construct
  source 1..45: mol_type = other DNA
  misc_feature 44..45: note = Phosphorothioate linkage
atctcatccctgcgtgtctccgactgcactgcctcagatcatgat

SEQ ID NO: 195 - DNA, 43 bp, synthetic construct
  source 1..43: mol_type = other DNA
  misc_feature 42..43: note = Phosphorothioate linkage
atctcatccctgcgtgtctccgactgcactgatcagtggcgat

SEQ ID NO: 196 - DNA, 45 bp, synthetic construct
  source 1..45: mol_type = other DNA
  misc_feature 44..45: note = Phosphorothioate linkage
atctcatccctgcgtgtctccgactgcactgctgcggagcatgat

SEQ ID NO: 197 - DNA, 45 bp, synthetic construct
  source 1..45: mol_type = other DNA
  misc_feature 44..45: note = Phosphorothioate linkage
atctcatccctgcgtgtctccgactgcacagtgctgtggcatgat

SEQ ID NO: 198 - DNA, 45 bp, synthetic construct
  source 1..45: mol_type = other DNA
  misc_feature 44..45: note = Phosphorothioate linkage
atctcatccctgcgtgtctccgactgcacacatatggcatatgat

SEQ ID NO: 199 - DNA, 43 bp, synthetic construct
  source 1..43: mol_type = other DNA
  misc_feature 42..43: note = Phosphorothioate linkage
atctcatccctgcgtgtctccgactgcacagatcgccacagat

SEQ ID NO: 200 - DNA, 43 bp, synthetic construct
  source 1..43: mol_type = other DNA
  misc_feature 42..43: note = Phosphorothioate linkage
atctcatccctgcgtgtctccgactgcacacggctcagatgat

SEQ ID NO: 201 - DNA, 43 bp, synthetic construct
  source 1..43: mol_type = other DNA
  misc_feature 42..43: note = Phosphorothioate linkage
atctcatccctgcgtgtctccgactgcacttgatgatagtgat

SEQ ID NO: 202 - DNA, 43 bp, synthetic construct
  source 1..43: mol_type = other DNA
  misc_feature 42..43: note = Phosphorothioate linkage
atctcatccctgcgtgtctccgactgcacaactgcacatagat

SEQ ID NO: 203 - DNA, 43 bp, synthetic construct
  source 1..43: mol_type = other DNA
  misc_feature 42..43: note = Phosphorothioate linkage
atctcatccctgcgtgtctccgactgcacgcatacatcctgat

SEQ ID NO: 204 - DNA, 45 bp, synthetic construct
  source 1..45: mol_type = other DNA
  misc_feature 44..45: note = Phosphorothioate linkage
atctcatccctgcgtgtctccgactgcacatctctggctgcagat

SEQ ID NO: 205 - DNA, 45 bp, synthetic construct
  source 1..45: mol_type = other DNA
  misc_feature 44..45: note = Phosphorothioate linkage
atctcatccctgcgtgtctccgactgcacatctggtgcatgtgat

SEQ ID NO: 206 - DNA, 43 bp, synthetic construct
  source 1..43: mol_type = other DNA
  misc_feature 42..43: note = Phosphorothioate linkage
atctcatccctgcgtgtctccgactgcacagcagcttcgcgat

SEQ ID NO: 207 - DNA, 45 bp, synthetic construct
  source 1..45: mol_type = other DNA
  misc_feature 44..45: note = Phosphorothioate linkage
atctcatccctgcgtgtctccgactgcacgcatatggcagcagat

SEQ ID NO: 208 - DNA, 43 bp, synthetic construct
  source 1..43: mol_type = other DNA
  misc_feature 42..43: note = Phosphorothioate linkage
atctcatccctgcgtgtctccgactgcacacagcaccgctgat

SEQ ID NO: 209 - DNA, 43 bp, synthetic construct
  source 1..43: mol_type = other DNA
  misc_feature 42..43: note = Phosphorothioate linkage
atctcatccctgcgtgtctccgactgcactagcatctggtgat

SEQ ID NO: 210 - DNA, 43 bp, synthetic construct
  source 1..43: mol_type = other DNA
  misc_feature 42..43: note = Phosphorothioate linkage
atctcatccctgcgtgtctccgactgcacttcagcatacagat

SEQ ID NO: 211 - DNA, 45 bp, synthetic construct
  source 1..45: mol_type = other DNA
  misc_feature 44..45: note = Phosphorothioate linkage
atctcatccctgcgtgtctccgactgcacatgcagatggcgagat

SEQ ID NO: 212 - DNA, 43 bp, synthetic construct
  source 1..43: mol_type = other DNA
  misc_feature 42..43: note = Phosphorothioate linkage
atctcatccctgcgtgtctccgactgcacagatctatccagat

SEQ ID NO: 213 - DNA, 45 bp, synthetic construct
  source 1..45: mol_type = other DNA
  misc_feature 44..45: note = Phosphorothioate linkage
atctcatccctgcgtgtctccgactgcacttcatgcatctcagat

SEQ ID NO: 214 - DNA, 45 bp, synthetic construct
  source 1..45: mol_type = other DNA
  misc_feature 44..45: note = Phosphorothioate linkage
atctcatccctgcgtgtctccgactgcacatgcaagtgtgatgat

SEQ ID NO: 215 - DNA, 43 bp, synthetic construct
  source 1..43: mol_type = other DNA
  misc_feature 42..43: note = Phosphorothioate linkage
atctcatccctgcgtgtctccgactgcactgttcgctgcagat

SEQ ID NO: 216 - DNA, 45 bp, synthetic construct
  source 1..45: mol_type = other DNA
  misc_feature 44..45: note = Phosphorothioate linkage
atctcatccctgcgtgtctccgactgcactcagatcctgcatgat

SEQ ID NO: 217 - DNA, 43 bp, synthetic construct
  source 1..43: mol_type = other DNA
  misc_feature 42..43: note = Phosphorothioate linkage
atctcatccctgcgtgtctccgactgcacacacagataatgat

SEQ ID NO: 218 - DNA, 43 bp, synthetic construct
  source 1..43: mol_type = other DNA
  misc_feature 42..43: note = Phosphorothioate linkage
atctcatccctgcgtgtctccgactgcacgatgctctggcgat

SEQ ID NO: 219 - DNA, 45 bp, synthetic construct
  source 1..45: mol_type = other DNA
  misc_feature 44..45: note = Phosphorothioate linkage
atctcatccctgcgtgtctccgactgcacagatccatcatctgat

SEQ ID NO: 220 - DNA, 45 bp, synthetic construct
  source 1..45: mol_type = other DNA
  misc_feature 44..45: note = Phosphorothioate linkage
atctcatccctgcgtgtctccgactgcactgcaccatcatatgat

SEQ ID NO: 221 - DNA, 45 bp, synthetic construct
  source 1..45: mol_type = other DNA
  misc_feature 44..45: note = Phosphorothioate linkage
atctcatccctgcgtgtctccgactgcacatgctatagcaatgat

SEQ ID NO: 222 - DNA, 45 bp, synthetic construct
  source 1..45: mol_type = other DNA
  misc_feature 44..45: note = Phosphorothioate linkage
atctcatccctgcgtgtctccgactgcacatctgcacatggcgat

SEQ ID NO: 223 - DNA, 45 bp, synthetic construct
  source 1..45: mol_type = other DNA
  misc_feature 44..45: note = Phosphorothioate linkage
atctcatccctgcgtgtctccgactgcacgctgcatgaagatgat

SEQ ID NO: 224 - DNA, 45 bp, synthetic construct
  source 1..45: mol_type = other DNA
  misc_feature 44..45: note = Phosphorothioate linkage
atctcatccctgcgtgtctccgactgcactattcagatgcatgat

SEQ ID NO: 225 - DNA, 43 bp, synthetic construct
  source 1..43: mol_type = other DNA
  misc_feature 42..43: note = Phosphorothioate linkage
atctcatccctgcgtgtctccgactgcactcgcgcaatgcgat

SEQ ID NO: 226 - DNA, 45 bp, synthetic construct
  source 1..45: mol_type = other DNA
  misc_feature 44..45: note = Phosphorothioate linkage
atctcatccctgcgtgtctccgactgcacgcgcagatggcatgat

SEQ ID NO: 227 - DNA, 43 bp, synthetic construct
  source 1..43: mol_type = other DNA
  misc_feature 42..43: note = Phosphorothioate linkage
atctcatccctgcgtgtctccgactgcacagtgaatcatcgat

SEQ ID NO: 228 - DNA, 43 bp, synthetic construct
  source 1..43: mol_type = other DNA
  misc_feature 42..43: note = Phosphorothioate linkage
atctcatccctgcgtgtctccgactgcacatgcaacactagat

SEQ ID NO: 229 - DNA, 43 bp, synthetic construct
  source 1..43: mol_type = other DNA
  misc_feature 42..43: note = Phosphorothioate linkage
atctcatccctgcgtgtctccgactgcactattatgatatgat

SEQ ID NO: 230 - DNA, 43 bp, synthetic construct
  source 1..43: mol_type = other DNA
  misc_feature 42..43: note = Phosphorothioate linkage
atctcatccctgcgtgtctccgactgcactcagaagcatcgat

SEQ ID NO: 231 - DNA, 43 bp, synthetic construct
  source 1..43: mol_type = other DNA
  misc_feature 42..43: note = Phosphorothioate linkage
atctcatccctgcgtgtctccgactgcacttcatatctgagat

SEQ ID NO: 232 - DNA, 43 bp, synthetic construct
  source 1..43: mol_type = other DNA
  misc_feature 42..43: note = Phosphorothioate linkage
atctcatccctgcgtgtctccgactgcacaatagctatgagat

SEQ ID NO: 233 - DNA, 45 bp, synthetic construct
  source 1..45: mol_type = other DNA
  misc_feature 44..45: note = Phosphorothioate linkage
atctcatccctgcgtgtctccgactgcacatggatagctgcagat

SEQ ID NO: 234 - DNA, 43 bp, synthetic construct
  source 1..43: mol_type = other DNA
  misc_feature 42..43: note = Phosphorothioate linkage
atctcatccctgcgtgtctccgactgcacataatatctgcgat

SEQ ID NO: 235 - DNA, 45 bp, synthetic construct
  source 1..45: mol_type = other DNA
  misc_feature 44..45: note = Phosphorothioate linkage
atctcatccctgcgtgtctccgactgcacgcatgcatcctgagat

SEQ ID NO: 236 - DNA, 43 bp, synthetic construct
  source 1..43: mol_type = other DNA
  misc_feature 42..43: note = Phosphorothioate linkage
atctcatccctgcgtgtctccgactgcacagcacagaggagat

SEQ ID NO: 237 - DNA, 43 bp, synthetic construct
  source 1..43: mol_type = other DNA
  misc_feature 42..43: note = Phosphorothioate linkage
atctcatccctgcgtgtctccgactgcacaactgcgctctgat

SEQ ID NO: 238 - DNA, 43 bp, synthetic construct
  source 1..43: mol_type = other DNA
  misc_feature 42..43: note = Phosphorothioate linkage
atctcatccctgcgtgtctccgactgcacacaagatgacagat

SEQ ID NO: 239 - DNA, 43 bp, synthetic construct
  source 1..43: mol_type = other DNA
  misc_feature 42..43: note = Phosphorothioate linkage
atctcatccctgcgtgtctccgactgcacagatgacattagat

SEQ ID NO: 240 - DNA, 45 bp, synthetic construct
  source 1..45: mol_type = other DNA
  misc_feature 44..45: note = Phosphorothioate linkage
atctcatccctgcgtgtctccgactgcacatggctcacatatgat

SEQ ID NO: 241 - DNA, 45 bp, synthetic construct
  source 1..45: mol_type = other DNA
  misc_feature 44..45: note = Phosphorothioate linkage
atctcatccctgcgtgtctccgactgcacaagagatgcatctgat

SEQ ID NO: 242 - DNA, 43 bp, synthetic construct
  source 1..43: mol_type = other DNA
  misc_feature 42..43: note = Phosphorothioate linkage
atctcatccctgcgtgtctccgactgcacatgaccgctgtgat

SEQ ID NO: 243 - DNA, 43 bp, synthetic construct
  source 1..43: mol_type = other DNA
  misc_feature 42..43: note = Phosphorothioate linkage
atctcatccctgcgtgtctccgactgcacgcatctggcgcgat

SEQ ID NO: 244 - DNA, 43 bp, synthetic construct
  source 1..43: mol_type = other DNA
  misc_feature 42..43: note = Phosphorothioate linkage
atctcatccctgcgtgtctccgactgcacttagctgtgatgat

SEQ ID NO: 245 - DNA, 43 bp, synthetic construct
  source 1..43: mol_type = other DNA
  misc_feature 42..43: note = Phosphorothioate linkage
atctcatccctgcgtgtctccgactgcactcgcaatagatgat

SEQ ID NO: 246 - DNA, 43 bp, synthetic construct
  source 1..43: mol_type = other DNA
  misc_feature 42..43: note = Phosphorothioate linkage
atctcatccctgcgtgtctccgactgcacactctctcaatgat

SEQ ID NO: 247 - DNA, 45 bp, synthetic construct
  source 1..45: mol_type = other DNA
  misc_feature 44..45: note = Phosphorothioate linkage
atctcatccctgcgtgtctccgactgcacactgcctgatgatgat

SEQ ID NO: 248 - DNA, 43 bp, synthetic construct
  source 1..43: mol_type = other DNA
  misc_feature 42..43: note = Phosphorothioate linkage
atctcatccctgcgtgtctccgactgcactctattcatatgat

SEQ ID NO: 249 - DNA, 43 bp, synthetic construct
  source 1..43: mol_type = other DNA
  misc_feature 42..43: note = Phosphorothioate linkage
atctcatccctgcgtgtctccgactgcacttgagcgcactgat

SEQ ID NO: 250 - DNA, 43 bp, synthetic construct
  source 1..43: mol_type = other DNA
  misc_feature 42..43: note = Phosphorothioate linkage
atctcatccctgcgtgtctccgactgcacgctgatcaggcgat

SEQ ID NO: 251 - DNA, 43 bp, synthetic construct
  source 1..43: mol_type = other DNA
  misc_feature 42..43: note = Phosphorothioate linkage
atctcatccctgcgtgtctccgactgcacgctctgttgatgat

SEQ ID NO: 252 - DNA, 43 bp, synthetic construct
  source 1..43: mol_type = other DNA
  misc_feature 42..43: note = Phosphorothioate linkage
atctcatccctgcgtgtctccgactgcactcaacatatctgat

SEQ ID NO: 253 - DNA, 45 bp, synthetic construct
  source 1..45: mol_type = other DNA
  misc_feature 44..45: note = Phosphorothioate linkage
atctcatccctgcgtgtctccgactgcacatgcggcgcagcagat

SEQ ID NO: 254 - DNA, 43 bp, synthetic construct
  source 1..43: mol_type = other DNA
  misc_feature 42..43: note = Phosphorothioate linkage
atctcatccctgcgtgtctccgactgcacttgctgtgcgcgat

SEQ ID NO: 255 - DNA, 43 bp, synthetic construct
  source 1..43: mol_type = other DNA
  misc_feature 42..43: note = Phosphorothioate linkage
atctcatccctgcgtgtctccgactgcacatagcgttcatgat

SEQ ID NO: 256 - DNA, 43 bp, synthetic construct
  source 1..43: mol_type = other DNA
  misc_feature 42..43: note = Phosphorothioate linkage
atctcatccctgcgtgtctccgactgcacgcacatagcctgat

SEQ ID NO: 257 - DNA, 43 bp, synthetic construct
  source 1..43: mol_type = other DNA
  misc_feature 42..43: note = Phosphorothioate linkage
atctcatccctgcgtgtctccgactgcacttctcatgtcagat

SEQ ID NO: 258 - DNA, 45 bp, synthetic construct
  source 1..45: mol_type = other DNA
  misc_feature 44..45: note = Phosphorothioate linkage
atctcatccctgcgtgtctccgactgcacagccatctgctgagat

SEQ ID NO: 259 - DNA, 43 bp, synthetic construct
  source 1..43: mol_type = other DNA
  misc_feature 42..43: note = Phosphorothioate linkage
atctcatccctgcgtgtctccgactgcacactcagcgccagat

SEQ ID NO: 260 - DNA, 43 bp, synthetic construct
  source 1..43: mol_type = other DNA
  misc_feature 42..43: note = Phosphorothioate linkage
atctcatccctgcgtgtctccgactgcacaacatctgatagat

SEQ ID NO: 261 - DNA, 43 bp, synthetic construct
  source 1..43: mol_type = other DNA
  misc_feature 42..43: note = Phosphorothioate linkage
atctcatccctgcgtgtctccgactgcactgctccgcatcgat

SEQ ID NO: 262 - DNA, 43 bp, synthetic construct
  source 1..43: mol_type = other DNA
  misc_feature 42..43: note = Phosphorothioate linkage
atctcatccctgcgtgtctccgactgcactatcgcttgatgat

SEQ ID NO: 263 - DNA, 43 bp, synthetic construct
  source 1..43: mol_type = other DNA
  misc_feature 42..43: note = Phosphorothioate linkage
atctcatccctgcgtgtctccgactgcactcttctcagcagat

SEQ ID NO: 264 - DNA, 43 bp, synthetic construct
  source 1..43: mol_type = other DNA
  misc_feature 42..43: note = Phosphorothioate linkage
atctcatccctgcgtgtctccgactgcacatggtgtgcacgat

SEQ ID NO: 265 - DNA, 43 bp, synthetic construct
  source 1..43: mol_type = other DNA
  misc_feature 42..43: note = Phosphorothioate linkage
atctcatccctgcgtgtctccgactgcacatatcatcttagat

SEQ ID NO: 266 - DNA, 43 bp, synthetic construct
  source 1..43: mol_type = other DNA
  misc_feature 42..43: note = Phosphorothioate linkage
atctcatccctgcgtgtctccgactgcacggtgctgctatgat

SEQ ID NO: 267 - DNA, 43 bp, synthetic construct
  source 1..43: mol_type = other DNA
  misc_feature 42..43: note = Phosphorothioate linkage
atctcatccctgcgtgtctccgactgcactgtcagcctatgat

SEQ ID NO: 268 - DNA, 45 bp, synthetic construct
  source 1..45: mol_type = other DNA
  misc_feature 44..45: note = Phosphorothioate linkage
atctcatccctgcgtgtctccgactgcacatgatcacacaatgat

SEQ ID NO: 269 - DNA, 43 bp, synthetic construct; identical to SEQ ID NO: 77
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 270 - DNA, 45 bp, synthetic construct; identical to SEQ ID NO: 78
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 271 - DNA, 45 bp, synthetic construct; identical to SEQ ID NO: 79
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 272 - DNA, 43 bp, synthetic construct; identical to SEQ ID NO: 80
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 273 - DNA, 43 bp, synthetic construct; identical to SEQ ID NO: 81
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 274 - DNA, 43 bp, synthetic construct; identical to SEQ ID NO: 82
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 275 - DNA, 43 bp, synthetic construct; identical to SEQ ID NO: 83
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 276 - DNA, 45 bp, synthetic construct; identical to SEQ ID NO: 50
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 277 - DNA, 45 bp, synthetic construct; identical to SEQ ID NO: 85
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 278 - DNA, 43 bp, synthetic construct; identical to SEQ ID NO: 86
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 279 - DNA, 43 bp, synthetic construct; identical to SEQ ID NO: 87
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 280 - DNA, 43 bp, synthetic construct; identical to SEQ ID NO: 88
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 281 - DNA, 43 bp, synthetic construct; identical to SEQ ID NO: 89
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 282 - DNA, 45 bp, synthetic construct; identical to SEQ ID NO: 90
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 283 - DNA, 43 bp, synthetic construct; identical to SEQ ID NO: 91
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 284 - DNA, 45 bp, synthetic construct; identical to SEQ ID NO: 92
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 285 - DNA, 43 bp, synthetic construct; identical to SEQ ID NO: 93
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 286 - DNA, 43 bp, synthetic construct; identical to SEQ ID NO: 94
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 287 - DNA, 45 bp, synthetic construct; identical to SEQ ID NO: 95
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 288 - DNA, 43 bp, synthetic construct; identical to SEQ ID NO: 96
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 289 - DNA, 43 bp, synthetic construct; identical to SEQ ID NO: 97
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 290 - DNA, 45 bp, synthetic construct; identical to SEQ ID NO: 98
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 291 - DNA, 43 bp, synthetic construct; identical to SEQ ID NO: 99
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 292 - DNA, 43 bp, synthetic construct; identical to SEQ ID NO: 100
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 293 - DNA, 43 bp, synthetic construct; identical to SEQ ID NO: 101
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 294 - DNA, 45 bp, synthetic construct; identical to SEQ ID NO: 102
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 295 - DNA, 43 bp, synthetic construct; identical to SEQ ID NO: 103
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 296 - DNA, 43 bp, synthetic construct; identical to SEQ ID NO: 104
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 297 - DNA, 43 bp, synthetic construct; identical to SEQ ID NO: 105
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 298 - DNA, 43 bp, synthetic construct; identical to SEQ ID NO: 106
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 299 - DNA, 45 bp, synthetic construct; identical to SEQ ID NO: 107
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 300 - DNA, 43 bp, synthetic construct; identical to SEQ ID NO: 108
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 301 - DNA, 43 bp, synthetic construct; identical to SEQ ID NO: 109
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 302 - DNA, 43 bp, synthetic construct; identical to SEQ ID NO: 110
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 303 - DNA, 43 bp, synthetic construct; identical to SEQ ID NO: 111
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 304 - DNA, 45 bp, synthetic construct; identical to SEQ ID NO: 112
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 305 - DNA, 43 bp, synthetic construct; identical to SEQ ID NO: 113
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 306 - DNA, 43 bp, synthetic construct; identical to SEQ ID NO: 114
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 307 - DNA, 43 bp, synthetic construct; identical to SEQ ID NO: 115
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 308 - DNA, 45 bp, synthetic construct; identical to SEQ ID NO: 116
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 309 - DNA, 45 bp, synthetic construct; identical to SEQ ID NO: 117
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 310 - DNA, 45 bp, synthetic construct; identical to SEQ ID NO: 118
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 311 - DNA, 45 bp, synthetic construct; identical to SEQ ID NO: 119
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 312 - DNA, 43 bp, synthetic construct; identical to SEQ ID NO: 120
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 313 - DNA, 45 bp, synthetic construct; identical to SEQ ID NO: 121
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 314 - DNA, 45 bp, synthetic construct; identical to SEQ ID NO: 122
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 315 - DNA, 43 bp, synthetic construct; identical to SEQ ID NO: 123
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 316 - DNA, 43 bp, synthetic construct; identical to SEQ ID NO: 124
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 317 - DNA, 43 bp, synthetic construct; identical to SEQ ID NO: 125
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 318 - DNA, 45 bp, synthetic construct; identical to SEQ ID NO: 126
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 319 - DNA, 43 bp, synthetic construct; identical to SEQ ID NO: 127
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 320 - DNA, 45 bp, synthetic construct; identical to SEQ ID NO: 128
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 321 - DNA, 45 bp, synthetic construct; identical to SEQ ID NO: 129
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 322 - DNA, 45 bp, synthetic construct; identical to SEQ ID NO: 130
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 323 - DNA, 45 bp, synthetic construct; identical to SEQ ID NO: 131
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 324 - DNA, 43 bp, synthetic construct; identical to SEQ ID NO: 132
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 325 - DNA, 45 bp, synthetic construct; identical to SEQ ID NO: 133
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 326 - DNA, 45 bp, synthetic construct; identical to SEQ ID NO: 134
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 327 - DNA, 45 bp, synthetic construct; identical to SEQ ID NO: 135
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 328 - DNA, 45 bp, synthetic construct; identical to SEQ ID NO: 136
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 329 - DNA, 45 bp, synthetic construct; identical to SEQ ID NO: 137
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 330 - DNA, 43 bp, synthetic construct; identical to SEQ ID NO: 138
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 331 - DNA, 43 bp, synthetic construct; identical to SEQ ID NO: 139
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 332 - DNA, 43 bp, synthetic construct; identical to SEQ ID NO: 140
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 333 - DNA, 43 bp, synthetic construct; identical to SEQ ID NO: 141
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 334 - DNA, 43 bp, synthetic construct; identical to SEQ ID NO: 142
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 335 - DNA, 43 bp, synthetic construct; identical to SEQ ID NO: 143
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 336 - DNA, 43 bp, synthetic construct; identical to SEQ ID NO: 144
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 337 - DNA, 43 bp, synthetic construct; identical to SEQ ID NO: 145
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 338 - DNA, 45 bp, synthetic construct; identical to SEQ ID NO: 146
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 339 - DNA, 43 bp, synthetic construct; identical to SEQ ID NO: 147
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 340 - DNA, 43 bp, synthetic construct; identical to SEQ ID NO: 148
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 341 - DNA, 45 bp, synthetic construct; identical to SEQ ID NO: 149
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 342 - DNA, 43 bp, synthetic construct; identical to SEQ ID NO: 150
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 343 - DNA, 43 bp, synthetic construct; identical to SEQ ID NO: 151
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 344 - DNA, 43 bp, synthetic construct; identical to SEQ ID NO: 152
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 345 - DNA, 43 bp, synthetic construct; identical to SEQ ID NO: 153
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 346 - DNA, 43 bp, synthetic construct; identical to SEQ ID NO: 154
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 347 - DNA, 43 bp, synthetic construct; identical to SEQ ID NO: 155
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 348 - DNA, 43 bp, synthetic construct; identical to SEQ ID NO: 156
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 349 - DNA, 43 bp, synthetic construct; identical to SEQ ID NO: 157
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 350 - DNA, 45 bp, synthetic construct; identical to SEQ ID NO: 158
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 351 - DNA, 43 bp, synthetic construct; identical to SEQ ID NO: 159
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 352 - DNA, 45 bp, synthetic construct; identical to SEQ ID NO: 160
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 353 - DNA, 43 bp, synthetic construct; identical to SEQ ID NO: 161
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 354 - DNA, 43 bp, synthetic construct; identical to SEQ ID NO: 162
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 355 - DNA, 45 bp, synthetic construct; identical to SEQ ID NO: 163
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 356 - DNA, 43 bp, synthetic construct; identical to SEQ ID NO: 164
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 357 - DNA, 43 bp, synthetic construct; identical to SEQ ID NO: 165
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 358 - DNA, 43 bp, synthetic construct; identical to SEQ ID NO: 166
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 359 - DNA, 43 bp, synthetic construct; identical to SEQ ID NO: 167
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 360 - DNA, 43 bp, synthetic construct; identical to SEQ ID NO: 168
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 361 - DNA, 43 bp, synthetic construct; identical to SEQ ID NO: 169
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 362 - DNA, 45 bp, synthetic construct; identical to SEQ ID NO: 170
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 363 - DNA, 43 bp, synthetic construct; identical to SEQ ID NO: 171
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 364 - DNA, 43 bp, synthetic construct; identical to SEQ ID NO: 172
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 365 - DNA, 43 bp, synthetic construct; identical to SEQ ID NO: 173
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 366 - DNA, 43 bp, synthetic construct; identical to SEQ ID NO: 174
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 367 - DNA, 45 bp, synthetic construct; identical to SEQ ID NO: 175
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 368 - DNA, 43 bp, synthetic construct; identical to SEQ ID NO: 176
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 369 - DNA, 45 bp, synthetic construct; identical to SEQ ID NO: 177
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 370 - DNA, 43 bp, synthetic construct; identical to SEQ ID NO: 178
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 371 - DNA, 43 bp, synthetic construct; identical to SEQ ID NO: 179
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 372 - DNA, 45 bp, synthetic construct; identical to SEQ ID NO: 180
  source 1..45: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 373 - DNA, 45 bp, synthetic construct; identical to SEQ ID NO: 181
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 374 - DNA, 43 bp, synthetic construct; identical to SEQ ID NO: 182
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 375 - DNA, 43 bp, synthetic construct; identical to SEQ ID NO: 183
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 376 - DNA, 45 bp, synthetic construct; identical to SEQ ID NO: 184
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 377 - DNA, 43 bp, synthetic construct; identical to SEQ ID NO: 185
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 378 - DNA, 45 bp, synthetic construct; identical to SEQ ID NO: 186
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 379 - DNA, 43 bp, synthetic construct; identical to SEQ ID NO: 187
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 380 - DNA, 45 bp, synthetic construct; identical to SEQ ID NO: 188
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 381 - DNA, 43 bp, synthetic construct; identical to SEQ ID NO: 51
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 382 - DNA, 43 bp, synthetic construct; identical to SEQ ID NO: 190
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 383 - DNA, 43 bp, synthetic construct; identical to SEQ ID NO: 191
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 384 - DNA, 43 bp, synthetic construct; identical to SEQ ID NO: 192
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 385 - DNA, 45 bp, synthetic construct; identical to SEQ ID NO: 193
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 386 - DNA, 45 bp, synthetic construct; identical to SEQ ID NO: 194
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 387 - DNA, 43 bp, synthetic construct; identical to SEQ ID NO: 195
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 388 - DNA, 45 bp, synthetic construct; identical to SEQ ID NO: 196
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 389 - DNA, 45 bp, synthetic construct; identical to SEQ ID NO: 197
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 390 - DNA, 45 bp, synthetic construct; identical to SEQ ID NO: 198
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 391 - DNA, 43 bp, synthetic construct; identical to SEQ ID NO: 199
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 392 - DNA, 43 bp, synthetic construct; identical to SEQ ID NO: 200
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 393 - DNA, 43 bp, synthetic construct; identical to SEQ ID NO: 201
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 394 - DNA, 43 bp, synthetic construct; identical to SEQ ID NO: 202
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 395 - DNA, 43 bp, synthetic construct; identical to SEQ ID NO: 203
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 396 - DNA, 45 bp, synthetic construct; identical to SEQ ID NO: 204
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 397 - DNA, 45 bp, synthetic construct; identical to SEQ ID NO: 205
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 398 - DNA, 43 bp, synthetic construct; identical to SEQ ID NO: 206
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 399 - DNA, 45 bp, synthetic construct; identical to SEQ ID NO: 207
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 400 - DNA, 43 bp, synthetic construct; identical to SEQ ID NO: 208
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 401 - DNA, 43 bp, synthetic construct; identical to SEQ ID NO: 209
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 402 - DNA, 43 bp, synthetic construct; identical to SEQ ID NO: 210
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 403 - DNA, 45 bp, synthetic construct; identical to SEQ ID NO: 211
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 404 - DNA, 43 bp, synthetic construct; identical to SEQ ID NO: 212
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 405 - DNA, 45 bp, synthetic construct; identical to SEQ ID NO: 213
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 406 - DNA, 45 bp, synthetic construct; identical to SEQ ID NO: 214
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 407 - DNA, 43 bp, synthetic construct; identical to SEQ ID NO: 215
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 408 - DNA, 45 bp, synthetic construct; identical to SEQ ID NO: 216
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 409 - DNA, 43 bp, synthetic construct; identical to SEQ ID NO: 217
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 410 - DNA, 43 bp, synthetic construct; identical to SEQ ID NO: 218
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 411 - DNA, 45 bp, synthetic construct; identical to SEQ ID NO: 219
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 412 - DNA, 45 bp, synthetic construct; identical to SEQ ID NO: 220
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 413 - DNA, 45 bp, synthetic construct; identical to SEQ ID NO: 221
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 414 - DNA, 45 bp, synthetic construct; identical to SEQ ID NO: 222
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 415 - DNA, 45 bp, synthetic construct; identical to SEQ ID NO: 223
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 416 - DNA, 45 bp, synthetic construct; identical to SEQ ID NO: 224
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 417 - DNA, 43 bp, synthetic construct; identical to SEQ ID NO: 225
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 418 - DNA, 45 bp, synthetic construct; identical to SEQ ID NO: 226
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 419 - DNA, 43 bp, synthetic construct; identical to SEQ ID NO: 227
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 420 - DNA, 43 bp, synthetic construct; identical to SEQ ID NO: 228
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 421 - DNA, 43 bp, synthetic construct; identical to SEQ ID NO: 229
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 422 - DNA, 43 bp, synthetic construct; identical to SEQ ID NO: 230
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 423 - DNA, 43 bp, synthetic construct; identical to SEQ ID NO: 231
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 424 - DNA, 43 bp, synthetic construct; identical to SEQ ID NO: 232
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 425 - DNA, 45 bp, synthetic construct; identical to SEQ ID NO: 233
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 426 - DNA, 43 bp, synthetic construct; identical to SEQ ID NO: 234
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 427 - DNA, 45 bp, synthetic construct; identical to SEQ ID NO: 235
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 428 - DNA, 43 bp, synthetic construct; identical to SEQ ID NO: 236
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 429 - DNA, 43 bp, synthetic construct; identical to SEQ ID NO: 237
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 430 - DNA, 43 bp, synthetic construct; identical to SEQ ID NO: 238
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 431 - DNA, 43 bp, synthetic construct; identical to SEQ ID NO: 239
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 432 - DNA, 45 bp, synthetic construct; identical to SEQ ID NO: 240
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 433 - DNA, 45 bp, synthetic construct; identical to SEQ ID NO: 241
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 434 - DNA, 43 bp, synthetic construct; identical to SEQ ID NO: 242
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 435 - DNA, 43 bp, synthetic construct; identical to SEQ ID NO: 243
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 436 - DNA, 43 bp, synthetic construct; identical to SEQ ID NO: 244
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 437 - DNA, 43 bp, synthetic construct; identical to SEQ ID NO: 245
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 438 - DNA, 43 bp, synthetic construct; identical to SEQ ID NO: 246
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 439 - DNA, 45 bp, synthetic construct; identical to SEQ ID NO: 247
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 440 - DNA, 43 bp, synthetic construct; identical to SEQ ID NO: 248
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 441 - DNA, 43 bp, synthetic construct; identical to SEQ ID NO: 249
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 442 - DNA, 43 bp, synthetic construct; identical to SEQ ID NO: 250
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 443 - DNA, 43 bp, synthetic construct; identical to SEQ ID NO: 251
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 444 - DNA, 43 bp, synthetic construct; identical to SEQ ID NO: 252
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 445 - DNA, 45 bp, synthetic construct; identical to SEQ ID NO: 253
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 446 - DNA, 43 bp, synthetic construct; identical to SEQ ID NO: 254
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 447 - DNA, 43 bp, synthetic construct; identical to SEQ ID NO: 255
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 448 - DNA, 43 bp, synthetic construct; identical to SEQ ID NO: 256
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 449 - DNA, 43 bp, synthetic construct; identical to SEQ ID NO: 257
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 450 - DNA, 45 bp, synthetic construct; identical to SEQ ID NO: 258
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 451 - DNA, 43 bp, synthetic construct; identical to SEQ ID NO: 259
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 452 - DNA, 43 bp, synthetic construct; identical to SEQ ID NO: 260
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 453 - DNA, 43 bp, synthetic construct; identical to SEQ ID NO: 261
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 454 - DNA, 43 bp, synthetic construct; identical to SEQ ID NO: 262
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 455 - DNA, 43 bp, synthetic construct; identical to SEQ ID NO: 263
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 456 - DNA, 43 bp, synthetic construct; identical to SEQ ID NO: 264
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 457 - DNA, 43 bp, synthetic construct; identical to SEQ ID NO: 265
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 458 - DNA, 43 bp, synthetic construct; identical to SEQ ID NO: 266
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 459 - DNA, 43 bp, synthetic construct; identical to SEQ ID NO: 267
  source 1..43: mol_type = other DNA
  modified_base 19: mod_base = OTHER; note = Deoxyuracil
  modified_base 25: mod_base = OTHER; note = Deoxyuracil
  misc_feature 42..43: note = Phosphorothioate linkage

SEQ ID NO: 460 - DNA, 45 bp, synthetic construct; identical to SEQ ID NO: 268
  source 1..45: mol_type = other DNA
  modified_base 19: mod_base = OTHER; note = Deoxyuracil
  modified_base 25: mod_base = OTHER; note = Deoxyuracil
  misc_feature 44..45: note = Phosphorothioate linkage

SEQ ID NO: 461 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcattcgagctgtgcagtcggagacacgcagggatgagatggt

SEQ ID NO: 462 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcgatggctgcacatgtgcagtcggagacacgcagggatgagatggt

SEQ ID NO: 463 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattcatgtgtgatgtgcagtcggagacacgcagggatgagatggt

SEQ ID NO: 464 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatgcctacacagtgcagtcggagacacgcagggatgagatggt

SEQ ID NO: 465 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagaggatacatgtgcagtcggagacacgcagggatgagatggt

SEQ ID NO: 466 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcataggctatatgtgcagtcggagacacgcagggatgagatggt

SEQ ID NO: 467 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgagcatgaatcgtgcagtcggagacacgcagggatgagatggt

SEQ ID NO: 468 - DNA, 47 bp, synthetic construct; identical to SEQ ID NO: 48
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil

SEQ ID NO: 469 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgcaggatgcgcgtgcagtcggagacacgcagggatgagatggt

SEQ ID NO: 470 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcattcgtgcagtgtgcagtcggagacacgcagggatgagatggt

SEQ ID NO: 471 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctggcagtgcgtgtgcagtcggagacacgcagggatgagatggt

SEQ ID NO: 472 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgtgctatggcagtgcagtcggagacacgcagggatgagatggt

SEQ ID NO: 473 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacagcacaactgtgcagtcggagacacgcagggatgagatggt

SEQ ID NO: 474 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgatcgcgccatgtgcagtcggagacacgcagggatgagatggt

SEQ ID NO: 475 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatgatatctaagtgcagtcggagacacgcagggatgagatggt

SEQ ID NO: 476 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgcgcacaggatgtgcagtcggagacacgcagggatgagatggt

SEQ ID NO: 477 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatgtgccacgtgtgcagtcggagacacgcagggatgagatggt

SEQ ID NO: 478 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacaggacgcatgtgcagtcggagacacgcagggatgagatggt

SEQ ID NO: 479 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctatgcagagcattgtgcagtcggagacacgcagggatgagatggt

SEQ ID NO: 480 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtgcaatgtagtgcagtcggagacacgcagggatgagatggt

SEQ ID NO: 481 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctggctcgctatgtgcagtcggagacacgcagggatgagatggt

SEQ ID NO: 482 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgcatgcactccgtgcagtcggagacacgcagggatgagatggt

SEQ ID NO: 483 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagtatctccatgtgcagtcggagacacgcagggatgagatggt

SEQ ID NO: 484 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgacaggctcagtgcagtcggagacacgcagggatgagatggt

SEQ ID NO: 485 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatcaatctcgagtgcagtcggagacacgcagggatgagatggt

SEQ ID NO: 486 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagatgagatggatgtgcagtcggagacacgcagggatgagatggt

SEQ ID NO: 487 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctggctgacagtgtgcagtcggagacacgcagggatgagatggt

SEQ ID NO: 488 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcagcaagtctgtgcagtcggagacacgcagggatgagatggt

SEQ ID NO: 489 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcctgctatatgtgcagtcggagacacgcagggatgagatggt

SEQ ID NO: 490 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatcttagacatgtgcagtcggagacacgcagggatgagatggt

SEQ ID NO: 491 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattcagctgcgatgtgcagtcggagacacgcagggatgagatggt

SEQ ID NO: 492 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcaatacagagtgcagtcggagacacgcagggatgagatggt

SEQ ID NO: 493 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgaatcgatgatgtgcagtcggagacacgcagggatgagatggt

SEQ ID NO: 494 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcatattcacagtgcagtcggagacacgcagggatgagatggt

SEQ ID NO: 495 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgagatcattcagtgcagtcggagacacgcagggatgagatggt

SEQ ID NO: 496 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctcatgcagcttctgtgcagtcggagacacgcagggatgagatggt

SEQ ID NO: 497 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcgcaatctagtgcagtcggagacacgcagggatgagatggt

SEQ ID NO: 498 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtgaggatgcgtgcagtcggagacacgcagggatgagatggt

SEQ ID NO: 499 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgatatgttgtgtgcagtcggagacacgcagggatgagatggt

SEQ ID NO: 500 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgcgatgcaggatgtgcagtcggagacacgcagggatgagatggt

SEQ ID NO: 501 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgagctgatgtgtgcagtcggagacacgcagggatgagatggt

SEQ ID NO: 502 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcgcattatatgcatgtgcagtcggagacacgcagggatgagatggt

SEQ ID NO: 503 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcgctgatgcattgagtgcagtcggagacacgcagggatgagatggt

SEQ ID NO: 504 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctagcacacattgtgcagtcggagacacgcagggatgagatggt

SEQ ID NO: 505 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagcatgaggctgagtgcagtcggagacacgcagggatgagatggt

SEQ ID NO: 506 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgcatgcgagagtgcagtcggagacacgcagggatgagatggt

SEQ ID NO: 507 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgctggctgctagtgcagtcggagacacgcagggatgagatggt

SEQ ID NO: 508 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacagtctggcagtgcagtcggagacacgcagggatgagatggt

SEQ ID NO: 509 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcaggagcagtgcgtgcagtcggagacacgcagggatgagatggt

SEQ ID NO: 510 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgtgctgccatgtgtgcagtcggagacacgcagggatgagatggt

SEQ ID NO: 511 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgatctgcaagtgtgcagtcggagacacgcagggatgagatggt

SEQ ID NO: 512 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgctgtggcatagtgcagtcggagacacgcagggatgagatggt

SEQ ID NO: 513 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctcatgctgatgttgtgcagtcggagacacgcagggatgagatggt

SEQ ID NO: 514 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgaatcactgcagtgcagtcggagacacgcagggatgagatggt

SEQ ID NO: 515 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgatgcaggtgctgtgcagtcggagacacgcagggatgagatggt

SEQ ID NO: 516 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgatagcataagtgcagtcggagacacgcagggatgagatggt

SEQ ID NO: 517 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgcagcacgcattgtgcagtcggagacacgcagggatgagatggt

SEQ ID NO: 518 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgcactgagatgtgcagtcggagacacgcagggatgagatggt

SEQ ID NO: 519 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgcgaagcacatgtgcagtcggagacacgcagggatgagatggt

SEQ ID NO: 520 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctggctgcactcatgtgcagtcggagacacgcagggatgagatggt

SEQ ID NO: 521 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagcacatgcttgagtgcagtcggagacacgcagggatgagatggt

SEQ ID NO: 522 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacattgactgtgtgcagtcggagacacgcagggatgagatggt

SEQ ID NO: 523 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcatcaagtgtgtgcagtcggagacacgcagggatgagatggt

SEQ ID NO: 524 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgaggctctatgtgcagtcggagacacgcagggatgagatggt

SEQ ID NO: 525 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcatgacacaagtgcagtcggagacacgcagggatgagatggt

SEQ ID NO: 526 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcgagtgcattgtgcagtcggagacacgcagggatgagatggt

SEQ ID NO: 527 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagcgctcataagtgcagtcggagacacgcagggatgagatggt

SEQ ID NO: 528 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtgcaatgacgtgcagtcggagacacgcagggatgagatggt

SEQ ID NO: 529 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgttgcagtgatgtgcagtcggagacacgcagggatgagatggt

SEQ ID NO: 530 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcgcatcgcatgcaagtgcagtcggagacacgcagggatgagatggt

SEQ ID NO: 531 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcttgcgcacgtgcagtcggagacacgcagggatgagatggt

SEQ ID NO: 532 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcattatgagatagtgcagtcggagacacgcagggatgagatggt

SEQ ID NO: 533 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagtgcatagccatgtgcagtcggagacacgcagggatgagatggt

SEQ ID NO: 534 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgctcattcatagtgcagtcggagacacgcagggatgagatggt

SEQ ID NO: 535 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgatggtgcatagtgcagtcggagacacgcagggatgagatggt

SEQ ID NO: 536 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcgcacattgtgtgcagtcggagacacgcagggatgagatggt

SEQ ID NO: 537 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagatagtcattgtgcagtcggagacacgcagggatgagatggt

SEQ ID NO: 538 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgagcagcggctgtgcagtcggagacacgcagggatgagatggt

SEQ ID NO: 539 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcattctgcgcgcgtgcagtcggagacacgcagggatgagatggt

SEQ ID NO: 540 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacgctgaggctgtgcagtcggagacacgcagggatgagatggt

SEQ ID NO: 541 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtatgatcaagtgcagtcggagacacgcagggatgagatggt

SEQ ID NO: 542 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgcacagcagagtgcagtcggagacacgcagggatgagatggt

SEQ ID NO: 543 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagcaatacactgtgcagtcggagacacgcagggatgagatggt

SEQ ID NO: 544 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcaggcagatgcacagtgcagtcggagacacgcagggatgagatggt

SEQ ID NO: 545 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagcaacatagagtgcagtcggagacacgcagggatgagatggt

SEQ ID NO: 546 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcagatagttgtgcagtcggagacacgcagggatgagatggt

SEQ ID NO: 547 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattcatgagatctgtgcagtcggagacacgcagggatgagatggt

SEQ ID NO: 548 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcactggatgatagtgcagtcggagacacgcagggatgagatggt

SEQ ID NO: 549 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcttgtagcagtgcagtcggagacacgcagggatgagatggt

SEQ ID NO: 550 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacgtgcatattgtgcagtcggagacacgcagggatgagatggt

SEQ ID NO: 551 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatcgcagaagtgtgcagtcggagacacgcagggatgagatggt

SEQ ID NO: 552 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctagatatgcttgtgcagtcggagacacgcagggatgagatggt

SEQ ID NO: 553 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgagctgtcattgtgcagtcggagacacgcagggatgagatggt

SEQ ID NO: 554 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatcaagctcatcagtgcagtcggagacacgcagggatgagatggt

SEQ ID NO: 555 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcaggtcatatgtgcagtcggagacacgcagggatgagatggt

SEQ ID NO: 556 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgatatcggctgtgcagtcggagacacgcagggatgagatggt

SEQ ID NO: 557 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcaactgactgtgcagtcggagacacgcagggatgagatggt

SEQ ID NO: 558 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatacatctgttgtgcagtcggagacacgcagggatgagatggt

SEQ ID NO: 559 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgcgcagatgagtgcagtcggagacacgcagggatgagatggt

SEQ ID NO: 560 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacgcatgatccgtgcagtcggagacacgcagggatgagatggt

SEQ ID NO: 561 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagagcatgcaatagtgcagtcggagacacgcagggatgagatggt

SEQ ID NO: 562 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcatcaggtagtgcagtcggagacacgcagggatgagatggt

SEQ ID NO: 563 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtgattatatgtgcagtcggagacacgcagggatgagatggt

SEQ ID NO: 564 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattagcgctgcatgtgcagtcggagacacgcagggatgagatggt

SEQ ID NO: 565 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagcactgcgccatgtgcagtcggagacacgcagggatgagatggt

SEQ ID NO: 566 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacacattctcagtgcagtcggagacacgcagggatgagatggt

SEQ ID NO: 567 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgtaccatgcatgtgcagtcggagacacgcagggatgagatggt

SEQ ID NO: 568 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctcctcgcatgcatgtgcagtcggagacacgcagggatgagatggt

SEQ ID NO: 569 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgtatgcagattgtgcagtcggagacacgcagggatgagatggt

SEQ ID NO: 570 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctggcgctcatgctgtgcagtcggagacacgcagggatgagatggt

SEQ ID NO: 571 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagatcagataagtgcagtcggagacacgcagggatgagatggt

SEQ ID NO: 572 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcacatgcgctggatgtgcagtcggagacacgcagggatgagatggt

SEQ ID NO: 573 - DNA, 45 bp, synthetic construct; identical to SEQ ID NO: 52
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil

SEQ ID NO: 574 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgatgtatgttgtgcagtcggagacacgcagggatgagatggt

SEQ ID NO: 575 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcatctagccgtgcagtcggagacacgcagggatgagatggt

SEQ ID NO: 576 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagcacataactgtgcagtcggagacacgcagggatgagatggt

SEQ ID NO: 577 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgctgctcggcagtgcagtcggagacacgcagggatgagatggt

SEQ ID NO: 578 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgatctgaggcagtgcagtcggagacacgcagggatgagatggt

SEQ ID NO: 579 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgccactgatcagtgcagtcggagacacgcagggatgagatggt

SEQ ID NO: 580 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgctccgcagcagtgcagtcggagacacgcagggatgagatggt

SEQ ID NO: 581 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgccacagcactgtgcagtcggagacacgcagggatgagatggt

SEQ ID NO: 582 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatatgccatatgtgtgcagtcggagacacgcagggatgagatggt

SEQ ID NO: 583 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtggcgatctgtgcagtcggagacacgcagggatgagatggt

SEQ ID NO: 584 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatctgagccgtgtgcagtcggagacacgcagggatgagatggt

SEQ ID NO: 585 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcactatcatcaagtgcagtcggagacacgcagggatgagatggt

SEQ ID NO: 586 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctatgtgcagttgtgcagtcggagacacgcagggatgagatggt

SEQ ID NO: 587 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcaggatgtatgcgtgcagtcggagacacgcagggatgagatggt

SEQ ID NO: 588 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgcagccagagatgtgcagtcggagacacgcagggatgagatggt

SEQ ID NO: 589 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcacatgcaccagatgtgcagtcggagacacgcagggatgagatggt

SEQ ID NO: 590 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcgaagctgctgtgcagtcggagacacgcagggatgagatggt

SEQ ID NO: 591 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgctgccatatgcgtgcagtcggagacacgcagggatgagatggt

SEQ ID NO: 592 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagcggtgctgtgtgcagtcggagacacgcagggatgagatggt

SEQ ID NO: 593 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcaccagatgctagtgcagtcggagacacgcagggatgagatggt

SEQ ID NO: 594 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtatgctgaagtgcagtcggagacacgcagggatgagatggt

SEQ ID NO: 595 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctcgccatctgcatgtgcagtcggagacacgcagggatgagatggt

SEQ ID NO: 596 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctggatagatctgtgcagtcggagacacgcagggatgagatggt

SEQ ID NO: 597 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgagatgcatgaagtgcagtcggagacacgcagggatgagatggt

SEQ ID NO: 598 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatcacacttgcatgtgcagtcggagacacgcagggatgagatggt

SEQ ID NO: 599 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgcagcgaacagtgcagtcggagacacgcagggatgagatggt

SEQ ID NO: 600 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgcaggatctgagtgcagtcggagacacgcagggatgagatggt

SEQ ID NO: 601 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcattatctgtgtgtgcagtcggagacacgcagggatgagatggt

SEQ ID NO: 602 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgccagagcatcgtgcagtcggagacacgcagggatgagatggt

SEQ ID NO: 603 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagatgatggatctgtgcagtcggagacacgcagggatgagatggt

SEQ ID NO: 604 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatatgatggtgcagtgcagtcggagacacgcagggatgagatggt

SEQ ID NO: 605 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgctatagcatgtgcagtcggagacacgcagggatgagatggt

SEQ ID NO: 606 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcgccatgtgcagatgtgcagtcggagacacgcagggatgagatggt

SEQ ID NO: 607 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatcttcatgcagcgtgcagtcggagacacgcagggatgagatggt

SEQ ID NO: 608 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgcatctgaatagtgcagtcggagacacgcagggatgagatggt

SEQ ID NO: 609 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcattgcgcgagtgcagtcggagacacgcagggatgagatggt

SEQ ID NO: 610 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatgccatctgcgcgtgcagtcggagacacgcagggatgagatggt

SEQ ID NO: 611 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgatgattcactgtgcagtcggagacacgcagggatgagatggt

SEQ ID NO: 612 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctagtgttgcatgtgcagtcggagacacgcagggatgagatggt

SEQ ID NO: 613 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatatcataatagtgcagtcggagacacgcagggatgagatggt

SEQ ID NO: 614 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgatgcttctgagtgcagtcggagacacgcagggatgagatggt

SEQ ID NO: 615 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcagatatgaagtgcagtcggagacacgcagggatgagatggt

SEQ ID NO: 616 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcatagctattgtgcagtcggagacacgcagggatgagatggt

SEQ ID NO: 617 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgcagctatccatgtgcagtcggagacacgcagggatgagatggt

SEQ ID NO: 618 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcagatattatgtgcagtcggagacacgcagggatgagatggt

SEQ ID NO: 619 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctcaggatgcatgcgtgcagtcggagacacgcagggatgagatggt

SEQ ID NO: 620 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctcctctgtgctgtgcagtcggagacacgcagggatgagatggt

SEQ ID NO: 621 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagagcgcagttgtgcagtcggagacacgcagggatgagatggt

SEQ ID NO: 622 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgtcatcttgtgtgcagtcggagacacgcagggatgagatggt

SEQ ID NO: 623 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctaatgtcatctgtgcagtcggagacacgcagggatgagatggt

SEQ ID NO: 624 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatatgtgagccatgtgcagtcggagacacgcagggatgagatggt

SEQ ID NO: 625 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcagatgcatctcttgtgcagtcggagacacgcagggatgagatggt

SEQ ID NO: 626 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcacagcggtcatgtgcagtcggagacacgcagggatgagatggt

SEQ ID NO: 627 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcgccagatgcgtgcagtcggagacacgcagggatgagatggt

SEQ ID NO: 628 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatcacagctaagtgcagtcggagacacgcagggatgagatggt

SEQ ID NO: 629 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatctattgcgagtgcagtcggagacacgcagggatgagatggt

SEQ ID NO: 630 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcattgagagagtgtgcagtcggagacacgcagggatgagatggt

SEQ ID NO: 631 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcatcatcaggcagtgtgcagtcggagacacgcagggatgagatggt

SEQ ID NO: 632 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatatgaatagagtgcagtcggagacacgcagggatgagatggt

SEQ ID NO: 633 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagtgcgctcaagtgcagtcggagacacgcagggatgagatggt

SEQ ID NO: 634 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcctgatcagcgtgcagtcggagacacgcagggatgagatggt

SEQ ID NO: 635 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatcaacagagcgtgcagtcggagacacgcagggatgagatggt

SEQ ID NO: 636 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcagatatgttgagtgcagtcggagacacgcagggatgagatggt

SEQ ID NO: 637 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctgctgcgccgcatgtgcagtcggagacacgcagggatgagatggt

SEQ ID NO: 638 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgcgcacagcaagtgcagtcggagacacgcagggatgagatggt

SEQ ID NO: 639 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatgaacgctatgtgcagtcggagacacgcagggatgagatggt

SEQ ID NO: 640 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcaggctatgtgcgtgcagtcggagacacgcagggatgagatggt

SEQ ID NO: 641 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgacatgagaagtgcagtcggagacacgcagggatgagatggt

SEQ ID NO: 642 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tctcagcagatggctgtgcagtcggagacacgcagggatgagatggt

SEQ ID NO: 643 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctggcgctgagtgtgcagtcggagacacgcagggatgagatggt

SEQ ID NO: 644 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctatcagatgttgtgcagtcggagacacgcagggatgagatggt

SEQ ID NO: 645 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgatgcggagcagtgcagtcggagacacgcagggatgagatggt

SEQ ID NO: 646 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatcaagcgatagtgcagtcggagacacgcagggatgagatggt

SEQ ID NO: 647 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctgctgagaagagtgcagtcggagacacgcagggatgagatggt

SEQ ID NO: 648 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcgtgcacaccatgtgcagtcggagacacgcagggatgagatggt

SEQ ID NO: 649 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tctaagatgatatgtgcagtcggagacacgcagggatgagatggt

SEQ ID NO: 650 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcatagcagcaccgtgcagtcggagacacgcagggatgagatggt

SEQ ID NO: 651 - DNA, 45 bp, synthetic construct
  source 1..45: mol_type = other DNA
  modified_base 37: mod_base = OTHER; note = Deoxyuracil
  modified_base 42: mod_base = OTHER; note = Deoxyuracil
  modified_base 45: mod_base = OTHER; note = Deoxyuracil
tcataggctgacagtgcagtcggagacacgcagggatgagatggt

SEQ ID NO: 652 - DNA, 47 bp, synthetic construct
  source 1..47: mol_type = other DNA
  modified_base 39: mod_base = OTHER; note = Deoxyuracil
  modified_base 44: mod_base = OTHER; note = Deoxyuracil
  modified_base 47: mod_base = OTHER; note = Deoxyuracil
tcattgtgtgatcatgtgcagtcggagacacgcagggatgagatggt

SEQ ID NO: 653 - DNA, 27 bp, synthetic construct
  source 1..27: mol_type = other DNA
atcgtcgcagctgacctacgatgtcat

SEQ ID NO: 654 - DNA, 27 bp, synthetic construct
  source 1..27: mol_type = other DNA
  modified_base 5: mod_base = OTHER; note = Deoxyuracil
  modified_base 8: mod_base = OTHER; note = Deoxyuracil
  modified_base 15: mod_base = OTHER; note = Deoxyuracil
  modified_base 18: mod_base = OTHER; note = Deoxyuracil
  modified_base 21: mod_base = OTHER; note = Deoxyuracil
  modified_base 24: mod_base = OTHER; note = Deoxyuracil
atgatattgtaggttagttgtgatgat

SEQ ID NO: 655 - DNA, 27 bp, synthetic construct
  source 1..27: mol_type = other DNA
  modified_base 3: mod_base = OTHER; note = Deoxyuracil
  modified_base 6: mod_base = OTHER; note = Deoxyuracil
  modified_base 8: mod_base = OTHER; note = Deoxyuracil
  modified_base 11: mod_base = OTHER; note = Deoxyuracil
  modified_base 15..16: mod_base = OTHER; note = Deoxyuracil
  modified_base 19: mod_base = OTHER; note = Deoxyuracil
  modified_base 25: mod_base = OTHER; note = Deoxyuracil
attgttgtagttgatttatgatgttat

SEQ ID NO: 656 - DNA, 33 bp, synthetic construct
  source 1..33: mol_type = other DNA
  modified_base 2: mod_base = OTHER; note = 5-methylcytosine
  modified_base 4: mod_base = OTHER; note = 5-methylcytosine
  modified_base 6: mod_base = OTHER; note = 5-methylcytosine
  modified_base 10..12: mod_base = OTHER; note = 5-methylcytosine
  modified_base 15: mod_base = OTHER; note = 5-methylcytosine
  modified_base 17: mod_base = OTHER; note = 5-methylcytosine
  modified_base 20: mod_base = OTHER; note = 5-methylcytosine
  modified_base 22: mod_base = OTHER; note = 5-methylcytosine
  modified_base 24: mod_base = OTHER; note = 5-methylcytosine
  modified_base 27..28: mod_base = OTHER; note = 5-methylcytosine
  modified_base 32: mod_base = OTHER; note = 5-methylcytosine
acactctttccctacacgacgctcttccgatct

SEQ ID NO: 657 - DNA, 33 bp, synthetic construct
  source 1..33: mol_type = other DNA
  modified_base 5: mod_base = OTHER; note = 5-methylcytosine
  modified_base 13: mod_base = OTHER; note = 5-methylcytosine
  modified_base 15: mod_base = OTHER; note = 5-methylcytosine
  modified_base 17: mod_base = OTHER; note = 5-methylcytosine
  modified_base 20: mod_base = OTHER; note = 5-methylcytosine
  modified_base 25: mod_base = OTHER; note = 5-methylcytosine
  modified_base 27..28: mod_base = OTHER; note = 5-methylcytosine
  modified_base 32: mod_base = OTHER; note = 5-methylcytosine
agatcggaagagcacacgtctgaactccagtca